(19) United States
(12) Patent Application Publication
(10) Pub. No.: US 2023/0142468 A1
(43) Pub. Date: May 11, 2023

(54) METHODS FOR GENERATING CELL SPHEROIDS

(71) Applicants: Immatics US, Inc., Houston, TX (US); Immatics Biotechnologies GmbH, Tuebingen (DE)

(72) Inventors: Inbar ALFAGUTER, Houston, TX (US); Sara YOUSEF, (US); Kathy-Ann SECKER, Tuebingen (US); Amir ALPERT, Houston, TX (US); Juliane PALMER, (US); Maike JAWORSKI, (US)

(21) Appl. No.: 18/053,165

(22) Filed: Nov. 7, 2022

Related U.S. Application Data
(60) Provisional application No. 63/342,382, filed on May 16, 2022.
(60) Provisional application No. 63/276,983, filed on Nov. 8, 2021.

(51) Int. Cl. C12N 5/00

(52) U.S. Cl. CPC C12N 5/0062; C12N 2533/90; C12N 2513/00

(57) ABSTRACT

The present disclosure relates to improved methods for generating multicellular spheroids. In embodiments, an improved method may comprise (a) providing cells capable of spheroid formation; (b) centrifuging the cells; (c) adding extracellular matrix to the cells of (b) to produce a cell suspension comprising a desired concentration of extracellular matrix; and (d) allowing at least one spheroid to form; wherein (b) is performed before (c). In embodiments, cells may be seeded on a tissue culture apparatus before extracellular matrix is added to the cells. In embodiments, spheroids may be characterized, analyzed, challenged, otherwise tested, or a combination thereof. In embodiments, cells may be seeded in a volume of media that is a fraction of a volume of media that is desired for characterization, analysis, challenging, otherwise testing, or a combination thereof.

### CROSS-REFERENCE TO RELATED APPLICATIONS

This application claims priority to U.S. Provisional Application No. 63/342,382, filed 16 May 2022 and U.S. Provisional Application No. 63/276,983, filed 8 Nov. 2021. Each of these applications is incorporated by reference in its entirety.

### BACKGROUND

### Field

The present disclosure relates to improved methods for generating three dimensional (3D) multicellular spheroids (spheroids) using a scaffold, such as but not limited to extracellular matrix. The disclosure further provides for spherical cell compounds and compositions thereof.

### Background

In vitro cell culture may be used as a model system to understand cells. Many studies have been performed using two dimensional (2D) monolayer cell culture. However, in vitro conditions, particularly in 2D culture, may be very different from the in vivo physiological environment, so it may be problematic to ascertain the applicability of in vitro observations to in vivo conditions or to whole tissues, organs, or organisms. As compared to 2D cell culture systems, 3D cell culture may more closely replicate the in vivo physiological environment of cells and may offer a higher degree of physiological relevance for in vitro studies. Thus, cells in 3D culture in vitro may provide a system having more physiological relevance compared to 2D monolayer culture in vitro.

3D cell culture may allow cells to interact with their environment, such as the media, the extracellular matrix, other cells, and added components for treatment and/or testing, such as, but not limited to, immunotherapeutics and drugs, in three dimensions in a more physiologically relevant manner than 2D culture systems. For at least this reason, 3D cell culture may be more physiologically relevant, as compared to 2D cell culture. 3D cell cultures may exhibit more physiologically relevant viability, proliferation, differentiation, cell death, morphology, and/or other characteristics, as compared to 2D cell culture systems. 3D cell cultures may also exhibit more physiologically relevant response to stimuli, metabolism generally, nutrient metabolism, metabolism of added components for treatment and/or testing, gene expression, trafficking and processing of materials, protein synthesis, cell-cell interaction, interaction with the environment, and/or other behaviors, functions, and responses, as compared to 2D cell culture systems. Cells in 3D culture may produce structures mimicking in vivo tissues.

A spheroid, or multicellular spheroid, is a 3D aggregate of cells cultured in vitro from tissue explants or biopsies, established cell cultures, other sources, or a combination thereof. Spheroids may form in various shapes, including but not limited to, round, mass shaped, grape-like, or stellate, depending on cell type(s). Also depending on cell type(s), cells may form spheroids that are compact spheroids, tight aggregates, or loose aggregates of cells. Round or approximately round, compact spheroids may be desirable, at least because they may more accurately mimic in vivo conditions, such as, but not limited to, cell-cell interactions, oxygen gradient(s), and nutrient gradient(s). As non-limiting examples, each may comprise a hypoxic core, which may be necrotic, a quiescent middle layer, and a proliferating outer layer. Because their mimicking of the natural cellular environment may be improved compared to cells in 2D culture, spheroids, particularly round, compact spheroids, may be useful tools in various applications, including, but not limited to, biological, physiological, and mechanistic assays and, as non-limiting examples, for investigating cell-cell interactions, for investigating interactions of cells with their environment, for investigating behavior of genetically engineered cells, for discovery and testing of drugs and other therapeutics, such as, but not limited to, immunotherapeutics, for studying tumor cells, for studying stem cells, for studying organ-specific cells, for tissue and implant analysis, regeneration, and engineering, and for studying various radiotherapies. Spheroids may be homogenous in cell type, comprising only one type of cell, or heterogenous in cell type, comprising two or more types of cells. Spheroids may be generated by any appropriate method, such as seeding or dispersal on a 3D artificial matrix, such as, but not limited to ECM. See, e.g., Han S J, et al., Challenges of applying multicellular tumor spheroids in preclinical phase. Cancer Cell Int. 2021 Mar. 4; 21(1):152; Oraiopoulou M E, et al., A 3D tumor spheroid model for the T98G Glioblastoma cell line phenotypic characterization. Tissue Cell. 2019 August; 59:39-43; Nürnberg E, et al., Routine Optical Clearing of 3D-Cell Cultures: Simplicity Forward. Front Mol Biosci. 2020 Feb. 21; 7:20; Grigalavicius M. et al., Photodynamic Efficacy of Cercosporin in 3D Tumor Cell Cultures, Photochemistry and Photobiology, 2020, 96: 699-707; 2020 and Selby M., et al., 3D Models of the NCI60 Cell Lines for Screening Oncology Compounds, SLAS Discov. 2017 June; 22(5):473-483; each of which is incorporated herein by reference in its entirety.

Important considerations in spheroid generation and potential applications may include uniformity in morphology, uniformity in shape (such as circularity), or lack thereof (eccentricity; eccentricity may define a range of roundness for an object and may eliminate objects that fall outside this range; eccentricity ranges from 0 to 1 with a perfect circle having a value of 0), uniformity in spheroid size, spheroid compactness, uniformity of spheroid compactness, and the formation and/or remaining of non-spheroid satellite cellular aggregates and/or cellular debris. Existing methods used for the generation of spheroids commonly yield cultures comprising a high percentage of spheroids with non-uniform morphology, eccentric and/or non-uniform shape, non-uniform size, a diffuse structure, non-uniform compactness, and/or a high number of non-spheroid cellular aggregates and/or amount of cellular debris. Cultures having such undesirable attributes may be unsuitable or less suitable for their desired use, as compared to cultures comprising spheroids having a more uniform morphology, spheroids having a more compact structure, spheroids having a lower level of shape eccentricity (a higher level of circularity or other shape uniformity), spheroids having a more uniform size, cultures comprising a lower percentage of non-spheroid cellular aggregates and/or cellular debris, or a combination thereof. Cultures having a high degree of undesirable attributes may be less usable or unusable for their intended purpose and may need to be discarded.

It is desirable to develop more efficient methods of generating spheroids. It is desirable to develop spheroid generation methods that yield cultures comprising spheroids having a more uniform morphology, spheroids having a more compact structure and/or uniformity in compactness, spheroids having a lower level of shape eccentricity (a higher level of circularity or other shape uniformity), spheroids having a more uniform size, cultures comprising a lower percentage of non-spheroid cellular aggregates and/or cellular debris, or a combination thereof.

### BRIEF SUMMARY

In an embodiment, the disclosure provide for circular and well-defined cellular spheroids generated using methods described herein. The spheroids may, among other characteristics, take on a more uniform shape than spheroids generated using common methods and protocols described herein.

In an embodiment, a method for generating cellular spheroids may comprise: (a) providing cells capable of spheroid formation; (b) centrifuging the cells; (c) adding extracellular matrix to the cells of (b) to produce a cell suspension comprising a desired concentration of extracellular matrix; and (d) allowing at least one spheroid to form.

In an embodiment, (b) may be performed before (c).

In an embodiment, the method may further comprise centrifuging the cell suspension of (c) before performing (d).

In an embodiment, the cells may comprise tumor cells, immortalized cells, primary cells, or combinations thereof.

In an embodiment, the method may comprise seeding the cells in a cell culture apparatus prior to performing (b).

In an embodiment, the cell culture apparatus may comprise at least one partition, and the at least one spheroid may comprise only one spheroid in the at least one partition.

In an embodiment, the at least one spheroid may comprise a more uniform morphology, a more compact structure, a lower level of shape eccentricity, more uniformity of compactness, more uniformity of size, or a combination thereof, as compared to spheroids generated without performing (b) before (c).

In an embodiment, the at least one spheroid may comprise a lower amount of cellular debris, a lower number of non-spheroid cellular aggregates, or a combination thereof, as compared to a spheroid generated without performing (b) before (c).

In an embodiment, at least two spheroids may be formed, and the at least two spheroids may comprise a more uniform morphology, a more compact structure, a lower level of shape eccentricity, more uniformity of compactness, more uniformity of size, or a combination thereof, as measured against each other, as compared to spheroids generated (i) without performing (b) before (c), (ii) without seeding the cells in a cell culture apparatus prior to performing (c), or as in (i) and (ii).

In an embodiment, the at least one spheroid may comprise a lower amount of cellular debris, a lower number of non-spheroid cellular aggregates, or a combination thereof, as compared to spheroids generated (i) without performing (b) before (c), (ii) without seeding the cells in a cell culture apparatus prior to prior to performing (c), or as in (i) and (ii).

In an embodiment, the at least one spheroid may have a circularity of approximately 0.1 to approximately 1.0, a circularity of approximately 0.1 to approximately 1.0, a circularity of approximately 0.2 to approximately 1.0, a circularity of approximately 0.4 to approximately 1.0, a circularity of approximately 0.5 to approximately 1.0, a circularity of approximately 0.6 to approximately 1.0, a circularity of approximately 0.7 to approximately 0.95, a circularity of approximately 0.7 to approximately 0.9, a circularity of approximately 0.8 to approximately 0.85, a circularity of at least approximately 0.3, a circularity of at least approximately 0.4, a circularity of at least approximately 0.45, a circularity of at least approximately 0.5, a circularity of at least approximately 0.55, a circularity of at least approximately 0.6, a circularity of at least approximately 0.65, a circularity of at least approximately 0.7, a circularity of at least approximately 0.75, a circularity of at least approximately 0.8, a circularity of at least approximately 0.85, a circularity of at least approximately 0.9, a circularity of at least approximately 0.95, a circularity of at least approximately 0.97, a circularity of at least approximately 0.98, a circularity of at least approximately 0.99, or a circularity of approximately 1.

In an embodiment, the at least one spheroid may have an eccentricity of approximately 0 to approximately 0.7, an eccentricity of approximately 0.6 to approximately 0.75, an eccentricity of approximately 0.5 to approximately 0.95, an eccentricity of approximately 0.5 to approximately 9, an eccentricity of approximately 0.4 to approximately 0.85, an eccentricity of approximately 0.3 to approximately 0.7, an eccentricity of approximately 0.2 to approximately 0.65, an eccentricity of approximately 0.1 to approximately 0.5, an eccentricity of approximately 0.1 to approximately 0.4, an eccentricity of approximately 0.1 to approximately 0.3, an eccentricity of approximately 0.1 to approximately 0.2, an eccentricity of approximately 0.1, an eccentricity of approximately 0.05, an eccentricity of approximately 0.02, an eccentricity of approximately 0.01, an eccentricity of approximately 0, an eccentricity of approximately 0.7 or less, an eccentricity of approximately 0.65 or less, an eccentricity of approximately 0.6 or less, an eccentricity of approximately 0.55 or less, an eccentricity of approximately 0.5 or less, an eccentricity of approximately 0.45 or less, an eccentricity of approximately 0.4 or less, an eccentricity of approximately 0.35 or less, an eccentricity of approximately 0.3 or less, an eccentricity of approximately 0.25 or less, an eccentricity of approximately 0.2 or less, an eccentricity of approximately 0.15 or less, an eccentricity of approximately 0.1 or less, an eccentricity of approximately 0.05 or less, an eccentricity of approximately 0.02 or less, an eccentricity of approximately 0.01 or less, or an eccentricity of approximately 0.

In an embodiment, the method may further comprise: characterizing, analyzing, challenging, otherwise testing the at least one spheroid, or a combination thereof.

In an embodiment, the characterizing, the analyzing, the challenging, or the otherwise testing may comprise: exposing the at least one spheroid to at least one drug or engineered cell, wherein the at least one spheroid expresses a reporter gene, and measuring the expression level of the reporter gene.

In an embodiment, the at least one spheroid is exposed to an immune suppressor.

In an embodiment, the at least one spheroid is exposed to an immune suppressor for up to 12 hours, up to 24 hours, up to 36 hours, up to 2 days, up to 3 days, up to 4 days, up to 5 days, up to 6 days, up to 7 days, up to 8 days, or up to 9 days.

In an embodiment, the at least one spheroid is exposed to an engineered cell, e.g., a T cell, after being exposed to an immune suppressor.

In an embodiment, the at least one spheroid may be exposed to an engineered cell, e.g., a T cell, for up to 2 days, up to 3 days, up to 4 days, up to 5 days, up to 6 days, up to 7 days, up to 8 days, or up to 9 days.

In an embodiment, the at least one engineered cell, e.g., a T cell, may be pre-incubated with an immune suppressor before the at least one spheroid is exposed to said at least one engineered cell.

In an embodiment, the at least one engineered cell, e.g., a T cell, may be pre-incubated with an immune suppressor for up to 6 hours, up to 8 hours, up to 10 hours, up to 12 hours, up to 16 hours, up to 20 hours or up to 24 hours, before the at least one spheroid is exposed to said at least one engineered cell.

In an embodiment, the immune suppressor referred to in the methods described herein is an inhibitory cytokine.

In an embodiment, the immune suppressor referred to in the methods described herein is selected from TGF-beta, adenosine, IL-4, IL-10, lactate, or combinations thereof.

In an embodiment, the desired concentration of extracellular matrix reagent may be about 1%-about 5%, about 1.5%-about 4.0%, about 2.0%-about 3%, about 1.75%-about 2.25%, or about 2.5%.

In embodiment, the desired concentration of extracellular matrix reagent may be measured volume/volume.

In an embodiment, the reporter gene may encode a fluorescent protein.

In an embodiment, measuring the expression level may comprise measuring the size of the fluorescence area of the at least one spheroid, measuring the fluorescence intensity of the at least one spheroid, measuring the eccentricity of the at least one spheroid, or combinations thereof.

In an embodiment, the size of the at least one spheroid may be greater, the fluorescence intensity of the at least one spheroid may be greater, the eccentricity of the at least one spheroid may be lesser, or combinations thereof, before exposing the at least one spheroid to at least one drug or engineered cell compared to after exposing the at least one spheroid to at least one drug or engineered cell, thereby indicating a cytotoxic activity of the at least one drug or engineered cell.

In an embodiment, the engineered cell in the methods described herein may comprise a T cell expressing an exogenous TCR and an exogenous CD8.

In an embodiment, the exogenous CD8 may comprise a CD8αβ heterodimer.

In an embodiment, the exogenous CD8 may comprise a CD8a homodimer.

In an embodiment, the T cell may comprise a CD8+ T cell.

In an embodiment, the T cell may comprise a CD4+ T cell.

In an embodiment, the at least one spheroid may have a size of about 400 μm to about 600 μm along at least one axis, and may reach this size within about 84 to about 108 hours after cell seeding.

In an embodiment, the at least one spheroid may have a size of about 500 μm along at least one axis, and may reach this size within about 96 hours after cell seeding.

In an embodiment, the at least one spheroid may have a form and/or structure that may more accurately mimic at least one in vivo cell-cell interaction, at least one in vivo oxygen gradient, at least one in vivo nutrient gradient, or combinations thereof, as compared to spheroid(s) generated without performing (b) before (c).

In an embodiment, the at least one spheroid may have a form and/or structure that may more accurately mimic at least one in vivo cell-cell interaction, at least one in vivo oxygen gradient, at least one in vivo nutrient gradient, or combinations thereof, as compared to spheroid(s) generated (i) without performing (b) before (c), (ii) without seeding the cells in a cell culture apparatus prior to performing (c), or as in (i) and (ii).

In an embodiment, a cellular spheroid produced by any of the above methods is provided. In an embodiment, a cellular spheroid produced using a protocol described herein is provided.

In an embodiment, a cellular spheroid produced by any of the above methods is provided.

In an embodiment, a method for generating cellular spheroids, may comprise: (a) providing cells capable of spheroid formation in a first volume of media that is a first fraction of a testing volume desired for characterizing, analyzing, challenging, otherwise testing of at least one spheroid, or a combination thereof; (b) centrifuging the cells; (c) adding to the cells of (b) extracellular matrix in a second volume that is a second fraction of the testing volume to produce a cell suspension comprising a desired concentration of extracellular matrix; and (d) allowing at least one spheroid to form; wherein (b) is performed before (c).

In an embodiment, (b) may be performed before (c).

In an embodiment, the method may further comprise centrifuging the cell suspension of (c) before performing (d).

In an embodiment, the cells may comprise tumor cells, immortalized cells, primary cells, or combinations thereof.

In an embodiment, the method may comprise seeding the cells in a cell culture apparatus prior to performing (b).

In an embodiment, the method may comprise adding media and/or a control and/or a test composition, in a volume sufficient to a produce the testing volume.

In an embodiment, the cell culture apparatus may comprise at least one partition, and the at least one spheroid may comprise only one spheroid in the at least one partition.

In an embodiment, the at least one spheroid may comprise a more uniform morphology, a more compact structure, a lower level of shape eccentricity, more uniformity of compactness, more uniformity of size, or a combination thereof, as compared to spheroids generated without performing (b) before (c).

In an embodiment, the at least one spheroid may comprise a lower amount of cellular debris, a lower number of non-spheroid cellular aggregates, or a combination thereof, as compared to a spheroid generated without performing (b) before (c).

In an embodiment, at least two spheroids may be formed, and the at least two spheroids may comprise a more uniform morphology, a more compact structure, a lower level of shape eccentricity, more uniformity of compactness, more uniformity of size, or a combination thereof, as measured against each other, as compared to spheroids generated (i) without performing (b) before (c), (ii) without seeding the cells in a cell culture apparatus prior to performing (c), or as in (i) and (ii).

In an embodiment, the at least one spheroid may comprise a lower amount of cellular debris, a lower number of non-spheroid cellular aggregates, or a combination thereof, as compared to spheroids generated (i) without performing (b) before (c), (ii) without seeding the cells in a cell culture apparatus prior to prior to performing (c), or as in (i) and (ii).

In an embodiment, the at least one spheroid may have a circularity of approximately 0.1 to approximately 1.0, a circularity of approximately 0.1 to approximately 1.0, a circularity of approximately 0.2 to approximately 1.0, a circularity of approximately 0.4 to approximately 1.0, a circularity of approximately 0.5 to approximately 1.0, a circularity of approximately 0.6 to approximately 1.0, a circularity of approximately 0.7 to approximately 0.95, a circularity of approximately 0.7 to approximately 0.9, a circularity of approximately 0.8 to approximately 0.85, a circularity of at least approximately 0.3, a circularity of at least approximately 0.4, a circularity of at least approximately 0.45, a circularity of at least approximately 0.5, a circularity of at least approximately 0.55, a circularity of at least approximately 0.6, a circularity of at least approximately 0.65, a circularity of at least approximately 0.7, a circularity of at least approximately 0.75, a circularity of at least approximately 0.8, a circularity of at least approximately 0.85, a circularity of at least approximately 0.9, a circularity of at least approximately 0.95, a circularity of at least approximately 0.97, a circularity of at least approximately 0.98, a circularity of at least approximately 0.99, or a circularity of approximately 1.

In an embodiment, the at least one spheroid may have an eccentricity of approximately 0 to approximately 0.7, an eccentricity of approximately 0.6 to approximately 0.75, an eccentricity of approximately 0.5 to approximately 0.95, an eccentricity of approximately 0.5 to approximately 9, an eccentricity of approximately 0.4 to approximately 0.85, an eccentricity of approximately 0.3 to approximately 0.7, an eccentricity of approximately 0.2 to approximately 0.65, an eccentricity of approximately 0.1 to approximately 0.5, an eccentricity of approximately 0.1 to approximately 0.4, an eccentricity of approximately 0.1 to approximately 0.3, an eccentricity of approximately 0.1 to approximately 0.2, an eccentricity of approximately 0.1, an eccentricity of approximately 0.05, an eccentricity of approximately 0.02, an eccentricity of approximately 0.01, an eccentricity of approximately 0, an eccentricity of approximately 0.7 or less, an eccentricity of approximately 0.65 or less, an eccentricity of approximately 0.6 or less, an eccentricity of approximately 0.55 or less, an eccentricity of approximately 0.5 or less, an eccentricity of approximately 0.45 or less, an eccentricity of approximately 0.4 or less, an eccentricity of approximately 0.35 or less, an eccentricity of approximately 0.3 or less, an eccentricity of approximately 0.25 or less, an eccentricity of approximately 0.2 or less, an eccentricity of approximately 0.15 or less, an eccentricity of approximately 0.1 or less, an eccentricity of approximately 0.05 or less, an eccentricity of approximately 0.02 or less, an eccentricity of approximately 0.01 or less, or an eccentricity of approximately 0.

In an embodiment, the method may further comprise: characterizing, analyzing, challenging, otherwise testing the at least one spheroid, or a combination thereof.

In an embodiment, the characterizing, the analyzing, the challenging, or the otherwise testing may comprise: exposing the at least one spheroid to at least one drug or engineered cell, wherein the at least one spheroid expresses a reporter gene, and measuring the expression level of the reporter gene.

In an embodiment, the at least one spheroid is exposed to an immune suppressor.

In an embodiment, the at least one spheroid is exposed to an immune suppressor for up to 12 hours, up to 24 hours, up to 36 hours, up to 2 days, up to 3 days, up to 4 days, up to 5 days, up to 6 days, up to 7 days, up to 8 days, or up to 9 days.

In an embodiment, the at least one spheroid is exposed to an engineered cell, e.g., a T cell, after being exposed to an immune suppressor.

In an embodiment, the at least one spheroid may be exposed to an engineered cell, e.g., a T cell, for up to 2 days, up to 3 days, up to 4 days, up to 5 days, up to 6 days, up to 7 days, up to 8 days, or up to 9 days.

In an embodiment, the at least one engineered cell, e.g., a T cell, may be pre-incubated with an immune suppressor before the at least one spheroid is exposed to said at least one engineered cell.

In an embodiment, the at least one engineered cell, e.g., a T cell, may be pre-incubated with an immune suppressor for up to 6 hours, up to 8 hours, up to 10 hours, up to 12 hours, up to 16 hours, up to 20 hours or up to 24 hours, before the at least one spheroid is exposed to said at least one engineered cell.

In an embodiment, the immune suppressor referred to in the methods described herein is an inhibitory cytokine.

In an embodiment, the immune suppressor referred to in the methods described herein is selected from TGF-beta, adenosine, IL-4, IL-10, lactate, or combinations thereof.

In an embodiment, the desired concentration of extracellular matrix reagent may be about 1%-about 5%, about 1.5%-about 4.0%, about 2.0%-about 3%, about 1.75%-about 2.25%, or about 2.5%.

In embodiment, the desired concentration of extracellular matrix reagent may be measured volume/volume.

In an embodiment, the reporter gene may encode a fluorescent protein.

In an embodiment, measuring the expression level may comprise measuring the size of the fluorescence area of the at least one spheroid, measuring the fluorescence intensity of the at least one spheroid, measuring the eccentricity of the at least one spheroid, or combinations thereof.

In an embodiment, the size of the at least one spheroid may be greater, the fluorescence intensity of the at least one spheroid may be greater, the eccentricity of the at least one spheroid may be lesser, or combinations thereof, before exposing the at least one spheroid to at least one drug or engineered cell compared to after exposing the at least one spheroid to at least one drug or engineered cell, thereby indicating a cytotoxic activity of the at least one drug or engineered cell.

In an embodiment, the engineered cell in the methods described herein may comprise a T cell expressing an exogenous TCR and an exogenous CD8.

In an embodiment, the exogenous CD8 may comprise a CD8αβ heterodimer.

In an embodiment, the exogenous CD8 may comprise a CD8α homodimer.

In an embodiment, the T cell may comprise a CD8+ T cell.

In an embodiment, the T cell may comprise a CD4+ T cell.

In an embodiment, the at least one spheroid may have a size of about 400 μm to about 600 μm along at least one axis, and may reach this size within about 84 to about 108 hours after cell seeding.

In an embodiment, the at least one spheroid may have a size of about 500 μm along at least one axis, and may reach this size within about 96 hours after cell seeding.

In an embodiment, the at least one spheroid may have a form and/or structure that may more accurately mimic at least one in vivo cell-cell interaction, at least one in vivo oxygen gradient, at least one in vivo nutrient gradient, or combinations thereof, as compared to spheroid(s) generated without performing (b) before (c).

In an embodiment, the at least one spheroid may have a form and/or structure that may more accurately mimic at least one in vivo cell-cell interaction, at least one in vivo oxygen gradient, at least one in vivo nutrient gradient, or combinations thereof, as compared to spheroid(s) generated (i) without performing (b) before (c), (ii) without seeding the cells in a cell culture apparatus prior to performing (c), or as in (i) and (ii).

In an embodiment, a cellular spheroid produced by any of the above methods is provided. In an embodiment, a cellular spheroid produced using a protocol described herein is provided.

In an embodiment, cellular spheroids produced by any of the above methods may be derived from primary tumor samples.

### BRIEF DESCRIPTION OF THE DRAWINGS

FIG. 1 shows representative images of A375-RFP cell spheroids 72 hours after seeding cells generated using either the common protocol or a new protocol (Protocol A) described herein. The first column of images shows wells containing spheroid culture produced using the common protocol, with the cells seeded with Matrigel® (row 1), Geltrex™ (row 2), or BME (row 3). The second column of images shows wells containing spheroid culture produced using a new protocol (Protocol A), with the cells seeded with Matrigel® (row 1), Geltrex™ (row 2), or BME (row 3).

FIG. 2A and FIG. 2B show graphs comparing A375-RFP cell spheroid cultures generated using either the common protocol (black bars) or a new protocol (Protocol A) (grey bars), 72 hours after seeding. FIG. 2A shows the percentage of wells that could successfully be acquired and analyzed by IncuCyte® software. FIG. 2B shows the eccentricity of the spheroids, calculated using IncuCyte® software. Asterisks represent statistical significance.

FIG. 3A and FIG. 3B show graphs comparing growth rate of A375-RFP cell spheroid cultures generated using either the common protocol (FIG. 3A) or a new protocol (Protocol A) (FIG. 3B). Cells were seeded as previously set forth, 1e3 (1,000) cells per well with Matrigel®, Geltrex™, or BME.

FIG. 4A and FIG. 4B show spheroids generated using with Matrigel®. As shown in FIG. 4A, on Day 0 of the assay, the cells were less compact and less round than at Day 4. As shown in FIG. 4B, by Day 4 of the assay, spheroids formed and reached the size of approximately 500 μm.

FIG. 5 shows a schematic of a killing assay that may be used to assess killing of spheroids by T cells.

FIGS. 6A and 6B show graphs of the Largest Red Object Area in μm² (y-axis) plotted against Time in Days (x-axis) for spheroids formed using PRAME-expressing UACC257-RFP tumor cells prepared using an adjusted new protocol (Protocol B) and challenged with CD3⁺ T cells that were transduced with one of R11KEA TCR WT (TCR), CD8αβ.TCR, or CD8α.TCR; or T cell media (RPMI with 10% Human albumin serum) or non-transduced T cells were added. FIG. 6A shows an Effector to Target ratio of 25:1, while FIG. 6B shows an Effector to Target ratio of 10:1. FIG. 6A and FIG. 6B show for 8 (FIG. 6A) or 11 (FIG. 6B) days after cell seeding.

FIGS. 7A and 7B show graphs of the Largest Red Object Area in μm² (y-axis) plotted against Time in Days (x-axis) for spheroids formed using PRAME-expressing UACC257-RFP tumor cells prepared using an adjusted new protocol (Protocol B) and challenged with CD8⁺ T cells that were transduced with one of R11KEA TCR WT (TCR), CD8αβ.TCR, or CD8α.TCR; or T cell media (RPMI with 10% Human albumin serum) or non-transduced T cells were added. FIG. 7A shows an Effector to Target ratio of 25:1, while FIG. 7B shows an Effector to Target ratio of 10:1.

FIG. 8A and FIG. 8B show representative images (FIG. 8A) or graphs of normalized spheroid size against time in hours (FIG. 8B) for spheroids formed using PRAME-expressing UACC257-RFP tumor cells prepared using an adjusted new protocol (Protocol B) and challenged with CD4+ (FIG. 8A) or with either CD3+, CD4+ or CD8+ T cells (FIG. 8B) that were transduced with one of R11KEA TCR WT (TCR), CD8αβ.TCR, or CD8α.TCR; or T cell culture media (RPMI with 10% Human albumin serum) or non-transduced T cells were added.

FIG. 9 shows a graph of the Largest Red Object Area in μm² (y-axis) against Time (in Days) (x-axis), for 10 days after cell seeding, of PRAME-expressing UACC257-RFP spheroids challenged with either CD4⁺ T cells transduced with CD8αβ.TCR (“UACC257 CD4) or CD4⁺ T cells transduced with CD8αβ.TCR (“UACC257 CD8).

FIGS. 10A-10D show comparisons of spheroid formation using T98G-RFP cells seeded with different ECM reagents. FIG. 10A shows the percentage of wells that could successfully be acquired and analyzed by IncuCyte® software. FIG. 10B shows average eccentricity of spheroids. FIG. 10C shows a graph of T98G-RFP spheroid formation. Mean Largest Brightfield Object Area in μm² (y-axis) is plotted against Time in Hours (x-axis) for 55 hours after cell seeding. As shown in FIG. 10C, spheroid formation and growth were similar for T98G-RFP cells with Matrigel®, Geltrex™, or BME. FIG. 10D shows images of T98G-RFP spheroids formed with Matrigel®, Geltrex™, or BME. Images were obtained using an IncuCyte® instrument using a 10×objective 48 hours after seeding the cells. The two arrows in FIG. 10D point to wells that could not be analyzed by the software; although the spheroids formed in these wells were highly compact and round, uncontrollable factors, which may be, as non-limiting examples, media contaminants, scratches on the plate, or a combination thereof, affected the calculations of eccentricity and the percentage of wells that could successfully be acquired and analyzed by IncuCyte® software.

FIG. 11A shows a graph of Normalized Spheroid Size (normalized to time 0) (y-axis) plotted against Time in Hours (x-axis) for A375-RFP spheroids formed with Matrigel®, Geltrex™, or BME. FIG. 11B shows images of A375-RFP spheroids formed with Matrigel®, Geltrex™, or BME 72 hours after seeding. FIG. 11C shows a graph of Normalized Spheroid Size (normalized to time 0) (y-axis) plotted against Time in Hours (x-axis) for T98G-RFP spheroids formed with Matrigel®, Geltrex™, or BME. FIG. 11D shows images of T98G-RFP spheroids formed with Matrigel®, Geltrex™, or BME. FIG. 11E shows a graph of Normalized Spheroid Size (normalized to time 0) (y-axis) plotted against Time in Hours (x-axis) for UACC257-RFP spheroids formed with Matrigel®, Geltrex™, or BME. FIG. 11F shows images of UACC257-RFP spheroids formed with Matrigel®, Geltrex™, or BME 72 hours after seeding.

FIG. 12A shows a graph of Largest Brightfield Object Area in μm² (y-axis) plotted against Time in Hours (x-axis) for 90 hours after cell seeding for A375-RFP spheroids formed with Matrigel® Lot #: 1077001 (Batch 1) or Matrigel® Lot #: 1074009 (Batch 2). FIG. 12B shows a table of eccentricity of A375-RFP spheroids formed with Matrigel® Lot #: 1077001 (Batch 1) or Matrigel® Lot #: 1074009 (Batch 2).

FIG. 12C shows a graph of Largest Brightfield Object Area in μm² (y-axis) plotted against Time in Hours (x-axis) for 90 hours after cell seeding for T98G-RFP spheroids formed with Matrigel® Lot #: 1077001 (Batch 1) or Matrigel® Lot #: 1074009 (Batch 2). FIG. 12D shows a table of eccentricity of T98G-RFP spheroids formed with Matrigel® Lot #: 1077001 (Batch 1) or Matrigel® Lot #: 1074009 (Batch 2).

FIG. 12E shows a graph of Largest Brightfield Object Area in μm² (y-axis) plotted against Time in Hours (x-axis) for 90 hours after cell seeding for UACC257-RFP spheroids formed with Matrigel® Lot #: 1077001 (Batch 1) or Matrigel® Lot #: 1074009 (Batch 2). FIG. 12F shows a table of eccentricity of UACC257-RFP spheroids formed with Matrigel® Lot #: 1077001 (Batch 1) or Matrigel® Lot #: 1074009 (Batch 2).

FIG. 13A shows images of A375-RFP spheroids formed with 100 μl or 200 μl media. FIG. 13B shows a graph Normalized Spheroid Size (normalized to time 0) (y-axis) plotted against Time in Hours (x-axis) for A375-RFP spheroids formed with 100 μl or 200 μl media. These data are the average of three independent experiments.

FIG. 13C shows images of T98G-RFP spheroids formed with 100 μl or 200 μl media. FIG. 13D shows a graph Normalized Spheroid Size (normalized to time 0) (y-axis) plotted against Time in Hours (x-axis) for T98G-RFP spheroids formed with 100 μl or 200 μl media. These data are the average of three independent experiments.

FIG. 13E shows images of UACC257-RFP spheroids formed with 100 μl or 200 μl media.

FIG. 13F shows a graph Normalized Spheroid Size (normalized to time 0) (y-axis) plotted against Time in Hours (x-axis) for UACC257-RFP spheroids formed with 100 μl or 200 μl media. These data are the average of two independent experiments.

FIG. 14A shows images of A375-RFP spheroids formed with 1% or 2.5% ECM reagent (Matrigel®).

FIG. 14B shows images of T98G-RFP spheroids formed with 1% or 2.5% ECM reagent (Matrigel®).

FIG. 14C shows images of UACC257-RFP spheroids formed with 1% or 2.5% ECM reagent (Matrigel®).

FIGS. 15A-15C show the formation of human normal hepatocyte spheroids using iCell® Hepatocytes 2.0 (iHH). Spheres were generated after 2D preculture and accutase detachment using Protocol B described herein with 2.5% Geltrex™. FIG. 15A shows representative images of iHH cell spheroids derived from either 3,000 or 6,000 cells 72 hours after seeding. The scale bar indicates 400 μm. FIG. 15B shows the eccentricity of the spheroids 72 hours after seeding cells, calculated using IncuCyte® software. FIG. 15C shows the formation of spheroids (Brightfield Object Total Area (μm²/Image)) over time (hours) reaching a compact and stable shape.

FIGS. 16A-16C show the formation of human normal cardiomyocyte spheroids using iCell® Cardiomyocytes² (iHCM). Spheres were generated directly after thawing using Protocol B described herein with 2.5% Geltrex™. FIG. 16A shows representative images of iHCM cell spheroids derived from 2,500 cells 72 hours after seeding. The scale bar indicates 400 μm. FIG. 16B shows the eccentricity of the spheroids 72 hours after seeding cells, calculated using IncuCyte® software. FIG. 16C shows the formation of spheroids (Brightfield Object Total Area (μm²/Image)) over time (hours) reaching a compact and stable shape.

FIGS. 17A-17C show the formation of human normal astrocyte and neuron spheroids using iCell® Astrocytes (iHA) and iCell® GABANeurons (iHN). Spheres were generated directly after thawing using Protocol B described herein with 2.5% Geltrex™. FIG. 17A shows representative images of iHA and iHN cell spheroids derived from either 2,500 or 5,000 cells or cells in co-culture 114 hours after seeding. The scale bar indicates 400 μm. FIG. 17B shows the eccentricity of the spheroids 114 hours after seeding cells, calculated using IncuCyte® software. FIG. 17C shows the formation of spheroids (Brightfield Object Total Area (μm²/Image)) over time (hours) reaching a compact and stable shape.

FIGS. 18A-18C show the formation of human normal renal spheroids using PromoCell® Human Renal Epithelial Cells (HREpC). Spheres were generated after 2D preculture and trypsin detachment using Protocol B described herein with 2.5% Geltrex™. FIG. 18A shows representative images of HREpC spheroids derived from either 3,000 or 6,000 cells 24 hours after seeding. The scale bar indicates 400 μm. FIG. 18B shows the eccentricity of the spheroids 24 hours after seeding cells, calculated using IncuCyte® software. FIG. 18C shows the formation of spheroids (Brightfield Object Total Area (μm²/Image)) over time (hours) reaching a compact and stable shape.

FIGS. 19A-19C show the formation of human normal coronary artery spheroids using PromoCell® Human Coronary Artery Endothelial Cells (HCAEC). Spheres were generated after 2D preculture and trypsin detachment using Protocol B described herein with 2.5% Geltrex™. FIG. 19A shows representative images of HREpC spheroids derived from either 3,000 or 6,000 cells 72 hours after seeding. The scale bar indicates 400 μm. FIG. 19B shows the eccentricity of the spheroids 72 hours after seeding cells, calculated using IncuCyte® software. FIG. 19C shows the formation of spheroids (Brightfield Object Total Area (μm²/Image)) over time (hours) reaching a compact and stable shape.

FIG. 20 shows an example of Protocol B of spheroid production according to an embodiment of the present disclosure.

FIG. 21 shows an example of Protocol B of spheroid production according to another embodiment of the present disclosure.

FIG. 22A shows images of unstained and stained spheroids according to one embodiment of the present disclosure.

FIG. 22B shows the fluorescence signal from stained iHH and iHCM cell spheroids according to one embodiment of the present disclosure.

FIG. 22C shows the size of unstained and stained iHH and iHCM cell spheroids according to one embodiment of the present disclosure.

FIG. 22D shows the eccentricity of unstained and stained iHH and iHCM cell spheroids according to one embodiment of the present disclosure.

FIG. 23 shows tumor cell spheroids produced by seeding cells in four different types of 96-well ultra-low attachment (ULA) plates (plates 1-4) according to one embodiment of the present disclosure.

FIG. 24 shows tumor cell spheroids generated by using the common protocol or Protocol B according to one embodiment of the present disclosure.

FIG. 25 shows spheroid killing by T cells expressing target-specific TCRs according to one embodiment of the present disclosure.

FIG. 26 shows an example of Protocol B of spheroid production according to an embodiment of the present disclosure.

FIG. 27 shows an example of Protocol B of spheroid production according to another embodiment of the present disclosure.

### DETAILED DESCRIPTION

In an aspect, the disclosure provides for spherical cellular aggregates and compositions produced by methods described herein. In another aspect, spheroid compositions described herein comprise a single uniform spheroid cellular aggregate devoid of satellite or non-uniform aggregates.

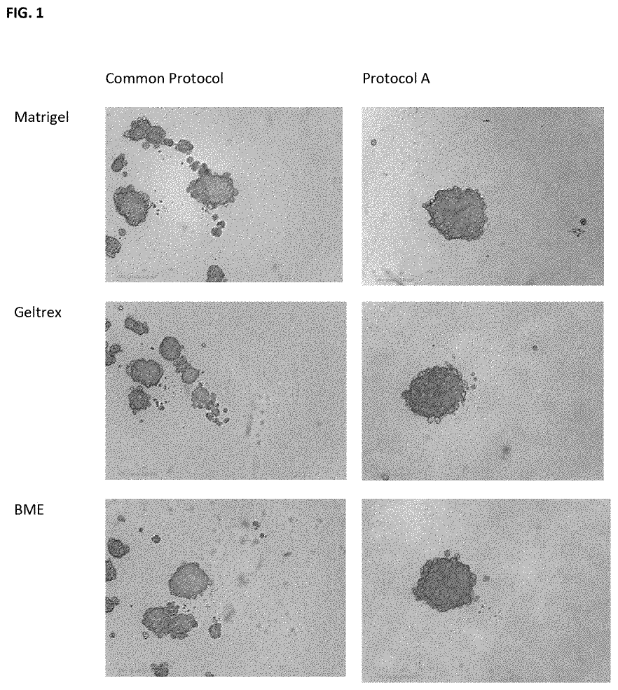

The disclosure provides for methods of generating spheroids characterized by the reduction of cell clusters and/or cell debris generated per analysed partition of a cell culture apparatus, such as a well of a 96-well culture plate. Spheroids described by the improved methods described herein may also have a more centralized location in a well with fewer or reduced satellite cell clusters and/or less cellular debris as compared to common protocols described herein. Spheroids described by the improved methods described herein may also have a more uniform morphology, a more compact structure and/or uniformity in compactness, a lower level of shape eccentricity (a higher level of circularity or other shape uniformity), a more uniform size, or a combination thereof, as compared to common protocols described herein. A representative analysis of improved spheroid formation is set forth in FIG. 1. The result of such improved cell spheroids may be improved, more streamlined, and/or more accurate analysis.

Spheroid size may be determined, as non-limiting examples, by measuring one, two, or more orthogonal diameters using an optical microscopy image. Area and volume may then be calculated using a diameter. See Han (2021) at page 9.

Eccentricity (“e”) may be calculated as

e=(1−(c²/a²))^{1/2}

Where “a” and “c” may be diameters of a spheroid. Eccentricity may also be calculated using automated methods, such as, but not limited to, calculation using IncuCyte® software. Eccentricity may range from 0 (for a perfect circle) to 1 (for an infinitely elongated polygon).

A non-limiting measure of lack of eccentricity, circularity may be calculated as

Circularity=4π×(area/perimeter²).

Id. At pages 8-9. Circularity may range from 0 (for an infinitely elongated polygon) to 1 (for a perfect circle). See id. At page 9.

Compactness may refer to density of cells of a spheroid. Morphology may refer to shape, size, compactness, cell composition, or combinations thereof, of a spheroid.

Spheroid uniformity may refer to uniformity of size, to uniformity of shape (e.g., circularity or lack of eccentricity), to uniformity of compactness, or to a combination thereof.

Spheroids may be generated via cell culture on various scaffold materials. As non-limiting examples, natural polymers, such as, but not limited to, gelatin, alginate, and collagen may also be employed as scaffold materials. As further non-limiting examples, synthetic polymers, such as, but not limited to, poly (lactic-co-glycolic) acid (PLGA) or polycaprolactone (PCL), and poly (ethylene glycol) (PEG), may also be employed as scaffold materials. In other non-limiting examples, spheroids may be generated via cell culture on various extra-cellular matrix (ECM) reagents, such as, but not limited to, those comprising extracellular matrix (ECM) proteins such as Laminin, Collagen IV, heparin sulfate proteoglycans, entactin/nidogen, or combinations thereof. ECM reagents may also comprise various growth factors. Non-limiting exemplary ECM reagents include reagents comprising solubilized basement membrane matrix secreted by Engelbreth-Holm-Swarm (EHS) mouse sarcoma cells (such as, but not limited to, Matrigel® Basement Membrane Matrix, which may be, as non-limiting examples, LDEV-free, reduced growth factor, or combinations thereof, produced by Corning Life Sciences (“Matrigel®”); Geltrex™ produced by Gibco® (“Geltrex™”); and Basement Membrane Extract produced by Trevigen® (“Cultrex®”), and other basement membrane extracts. See, e.g., Hughes C S, et al., Matrigel: a complex protein mixture required for optimal growth of cell culture. Proteomics. 2010 May; 10(9):1886-90, which is incorporated herein by reference in its entirety. Other formulations of Matrigel®, Geltrex™, and Cultrex® may be used, as well as other commercially available and lab-prepared basement membrane extracts. Such supporting ECM-like substrate products may be referred to herein as “extracellular matrix reagent” or “ECM reagent”. Concentrations of ECM referred to herein may refer to concentrations of ECM reagent. The percentage of ECM reagent may, as a non-limiting example, be calculated as a percentage of the volume of ECM reagent compared to the total volume of the media and cell suspension (v/v). The ECM reagent may be considered 100%, and may be diluted to comprise a percentage of the total volume per well. ECM, ECM reagent, media comprising ECM, media comprising ECM reagent, or combinations thereof, may be added to the cells to produce a desired culture concentration of extracellular matrix or extracellular matrix reagent. Unless otherwise specified, addition of ECM, ECM reagent, media comprising ECM, media comprising ECM reagent, or combinations thereof, may each individually or collectively be encompassed or referred to by the phrases “adding ECM”, “ECM is added”, or similar phrases.

Spheroids may be generated using many types of cells, including, but not limited to, non-mammalian and mammalian animal cells, including, but not limited to, human cells, murine cells, cells from domestic animals, and cells from wild animals. Non-limiting examples of cells that may be used to generate spheroids may include tumor cells of various lineages, other immortalized cells of various lineages, and primary cells of various types, such as, but not limited to, embryonic stem cells, pluripotent stem cells, multipotent stem cells, other stem cells, neural cells, hepatic cells, cardiac cells, arterial cells, renal cells, mammary cells, embryonic cells, prostate cells, and other cells, as well. Non-limiting examples of tumor cells that may be used to generate spheroids may include cells in the NCI-60 Human Tumor Cell Lines panel, solid tumor cells of various lineages, and liquid tumor cells of various lineages, and other types, as well. Tumor cells that may be used to generate spheroids may include, as non-limiting examples, tumor cells from explants, biopsies, or cultures, tumor cells that have spontaneously immortalized, and tumor cells that have been immortalized via engineering. Human normal primary cells may include cells differentiated from induced pluripotent stem cells (iPSC)-derived or isolated from normal human tissues, such as, but not limited to, adult tissues, tissues from children, fetal tissues, embryonic tissues, and/or placental tissues. One type of cell or a mixture of more than one type of cell may be utilized. Cells “capable of spheroid formation” are types of cells, or mixtures of more than one cell type, that form 3D multicellular spheroids upon being subjected to art-known methods of spheroid generation, improvements of such methods, new methods of spheroid generation (Protocol A), or adjusted new methods (Protocol B) of spheroid generation.

### A Common Method of Spheroid Production

In one commonly used method of spheroid generation (a common protocol), the following steps are performed, commonly in the following order:

ECM, ECM reagent, media comprising ECM, media comprising ECM reagent, or combinations thereof, may be added to cell suspension to produce a desired culture concentration of extracellular matrix or extracellular matrix reagent. Unless otherwise specified, addition of ECM, ECM reagent, media comprising ECM, media comprising ECM reagent, or combinations thereof, may each individually or collectively be encompassed or referred to by the phrases “adding ECM”, “ECM is added”, or similar phrases. Other scaffold materials may also be employed. A “desired culture concentration of extracellular matrix” may be that concentration of ECM or ECM reagent at which the type(s) of cell being used may generate spheroids. The concentration of extracellular matrix may be measured as the concentration of ECM reagent per well (such as, but not limited to, by v/v). The final concentration of is commonly approximately 2.5% of ECM reagent v/v, but may vary, such as, with different cell type(s). As a non-limiting example, approximately 2.5 μl of ECM reagent may be added to approximately 100 μl media.

The ECM reagent and the cell suspension are gently mixed.

The ECM cell suspension mixture is seeded in a tissue culture apparatus; commonly, approximately 200 μl per well is seeded in a 96 well ultra-low attachment (ULA) U-bottom plate (available from, e.g., Corning®, such as Corning® 7007 (330 μL total volume, 75 to 200 working volume). Approximately 1e3 (1,000) cells are commonly be seeded per well, but this parameter may vary with different cell type(s).

The ECM cell suspension mixture is centrifuged, commonly at approximately 125 Xg for approximately 10 minutes at approximately 4° C. These parameters may vary, such as, with different cell type(s).

The culture may be monitored for approximately 7-10 days, or for another desired period of time. A non-limiting example of a suitable cell monitoring process is monitoring using an IncuCyte® live cell imaging and analysis platform.

Media may be changed and/or characterization, analysis, challenging, and/or testing (such as addition of T cells) may be performed on day approximately 3, 4, or 5, or at another desired time or times.

As used herein, “a common protocol”, “the common protocol”, and/or the plural tenses of these phrases may refer to methods of spheroid generation comprising some or all of the above steps set forth in this section (A Common Method of Spheroid Production) in the order set forth and/or not comprising (i) centrifuging a cell suspension during the protocol before adding ECM, (ii) seeding a cell suspension during the protocol before adding ECM, or (iii) combinations of (i) and (ii).

### New Methods of Spheroid Production

In a new method of spheroid generation (representative new protocol (Protocol A), or improved methods), the following may be performed. In embodiments, (i) centrifuging a cell suspension during the protocol is performed before adding ECM, (ii) seeding a cell suspension during the protocol is performed before adding ECM, or (iii) combinations of (i) and (ii). In embodiments, the following are performed in the following order.

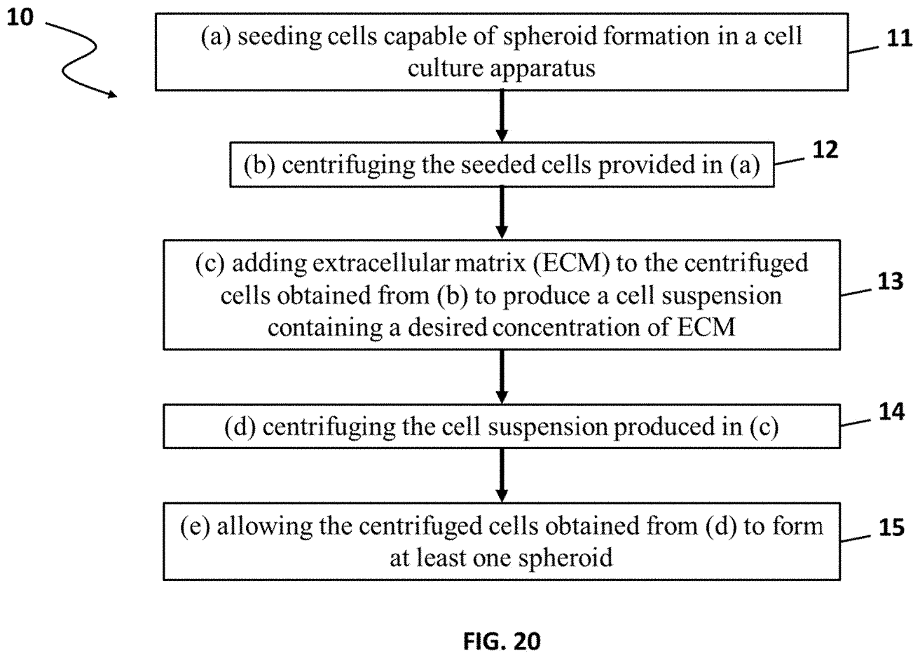

FIG. 20 shows an exemplary Protocol A of spheroid production according to one embodiment of the present disclosure. An exemplary Protocol A (10) may include (a) seeding cells capable of spheroid formation in a cell culture apparatus (11), (b) centrifuging the seeded cells provided in (a) (12), (c) adding extracellular matrix (ECM) to the centrifuged cells obtained from (b) to produce a cell suspension containing a desired concentration of ECM (13), (d) centrifuging the cell suspension produced in (c) (14), and (e) allowing the centrifuged cells obtained from (d) to form at least one spheroid (15).

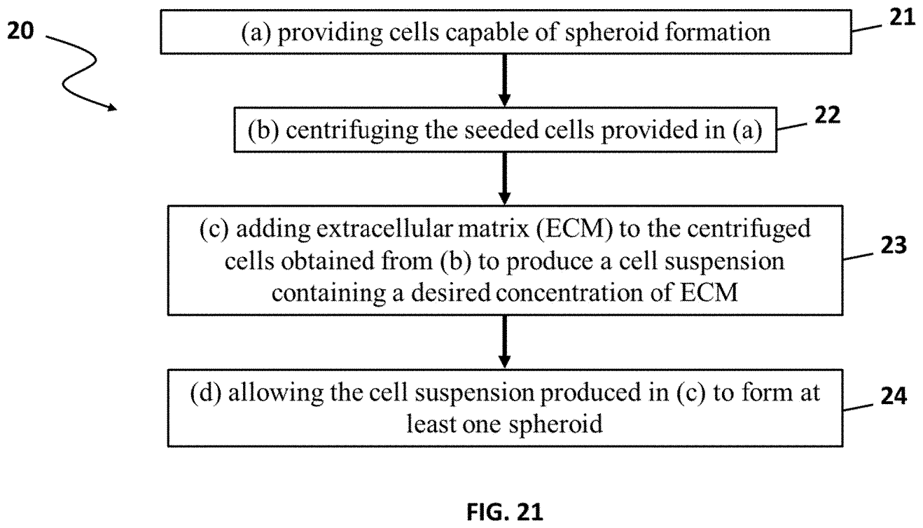

FIG. 21 shows an exemplary Protocol A of spheroid production according to another embodiment of the present disclosure. An exemplary Protocol A (20) may include (a) seeding cells capable of spheroid formation in a cell culture apparatus (21), (b) centrifuging the seeded cells provided in (a) (22), (c) adding extracellular matrix (ECM) to the centrifuged cells obtained from (b) to produce a cell suspension containing a desired concentration of ECM (23), and (d) allowing the cell suspension produced in (c) to form at least one spheroid (24).

Where cells are seeded before addition of ECM (as may be performed in a new protocol (Protocol A) as described herein), cell suspension may be seeded in a 96 well ULA U-bottom plate, such as a Corning® 7007 plate, in the amount of approximately 50 μl to approximately 100 approximately 100 or 50 μl to approximately 200 μl, of cell suspension per well. Approximately 5e2 (500) to approximately 1e4 (10,000), approximately 7.5e2 (750) to approximately 5e3 (5,000), approximately 1e3 (1,000) to approximately 2e3 (2,000), or approximately 1e3 (1,000) cells may be seeded per well. Other tissue culture plates or apparatus may be used. As non-limiting examples, 384-well or 1356-well plates may be used. As non-limiting examples, cell culture dishes, flasks, or tubes may be used. In embodiments, ULA tissue culture plates or apparatus are used. Other volumes of cell suspension, in accordance with suitability for the cell culture apparatus being used and/or the type(s) of cell being used and/or other considerations, may be used; and other cell concentrations, in accordance with suitability for the cell culture apparatus being used and/or the type(s) of cell being used and/or other considerations, may be used. Seeded cell density may be approximately 1×10⁴ to approximately 20×10⁶ cells/ml, approximately 1.5×10⁴ to approximately 10×10⁶ cells/ml, approximately 5×10⁴ to approximately 5×10⁶ cells/ml, approximately 1×10⁵ to approximately 5×10⁵ cells/ml, approximately 1×10⁴ to approximately 10×10⁴ cells/ml, approximately 1×10⁴ to approximately 10×10⁴ cells/ml, approximately 1.5×10⁴ to approximately 9×10⁴ cells/ml, approximately 1.5×10⁴ to approximately 5×10⁴ cells/ml, approximately 2×10⁴ to approximately 4×10⁴ cells/ml, approximately 2×10⁴ to approximately 3×10⁴ cells/ml, or approximately 2×10⁴ cells/ml as non-limiting examples. These densities may result in a final cell density in cells/ml of approximately one half of the seeded cell density, after ECM is added to the cells, but the number of cells/per well may remain the same. The cell seeding density per volume, number of cells per well, or combinations thereof may be optimized according to cell type and/or type of culture apparatus, as non-limiting examples. As another non-limiting example, cell seeding density per volume, number of cells per well, or combinations thereof may be optimized in applications where more than one type(s) of cell is used. The cell seeding density per volume, number of cells per well, number of cells per cell culture apparatus partition, or combinations thereof may be adjusted, such as but not limited to, to result in the growth of one spheroid per well of a size of approximately 500 μm by approximately Day 4 after seeding, which may, in embodiments, be achieved using a new protocol (Protocol A) as described herein. The cell seeding density per volume, number of cells per well, number of cells per cell culture apparatus partition, or combinations thereof may be adjusted, such as but not limited to, to result in a cell concentration desirable for characterization, analysis, challenging, and/or testing.

The cell suspension may be centrifuged at approximately 125 Xg for approximately 10 minutes at approximately 4° C. Other centrifugation relative centrifugal forces, other times, and other temperatures may be utilized, and these parameters may be optimized, as a non-limiting example, for the type(s) of cell being used. As non-limiting examples, cell suspensions may be centrifuged at approximately 25 Xg to approximately 400 Xg, at approximately 50 Xg to approximately 275 Xg, at approximately 75 Xg to approximately 250 Xg, at approximately 100 Xg to approximately 225 Xg, at approximately 105 Xg to approximately 215 Xg, at approximately 110 Xg to approximately 200 Xg, at approximately 115 Xg to approximately 175 Xg, at approximately 120 Xg to approximately 150 Xg, at approximately 150 Xg, at approximately 135 Xg, at approximately 130 Xg, or at approximately 125 Xg, at approximately 120 Xg or at approximately 115 Xg. As non-limiting examples, cell suspensions may be centrifuged for approximately 2 minutes to approximately 30 minutes, for approximately 4 minutes to approximately 20 minutes, for approximately 5 minutes to approximately 15 minutes, or for approximately 10 minutes. As non-limiting examples, cell suspensions may be centrifuged at approximately 1° C. to approximately 20° C., at approximately 3° C. to approximately 15° C., at approximately 4° C. to approximately 10° C., or at approximately 4° C. The cells suspension may be centrifuged before adding ECM.

ECM, ECM reagent, media comprising ECM, media comprising ECM reagent, or combinations thereof, may be added to the cells to produce a desired culture concentration of extracellular matrix or extracellular matrix reagent. Unless otherwise specified, addition of ECM, ECM reagent, media comprising ECM, media comprising ECM reagent, or combinations thereof, may each individually or collectively be encompassed or referred to by the phrases “adding ECM”, “ECM is added”, or similar phrases. Other scaffold materials may also be employed. A “desired culture concentration of extracellular matrix” may be that concentration of ECM or ECM reagent at which the type(s) of cell being used generates spheroids. As a non-limiting example, where a 96 well ULA U-bottom plate with approximately 50 μl of cell suspension per well is used, the amount of media with ECM reagent added may be approximately 50 μl. As another non-limiting example, where a 96 well ULA U-bottom plate with approximately 100 μl of cell suspension per well is used, the amount of media with ECM reagent added may be approximately 100 μl. The final concentration of ECM reagent may be approximately 2.5% of the final volume, which may be measured v/v. Other concentrations of ECM or ECM reagent may be used, and the concentration may be optimized for the type(s) of cell being used. As non-limiting examples, a concentration of extracellular matrix reagent v/v may be approximately 1.5%-approximately 4.0%, may be approximately 1.75%-approximately 2.25%, may be approximately 2.0%-approximately 3.0%, or may be approximately 2.5%, which may, as a non-limiting example, be measured v/v. As a non-limiting example, approximately 2.5 μl of ECM reagent may be added to approximately 100 μl media. Concentration of ECM may be adjusted, as non-limiting examples, for different cell type(s), to improve spheroid compactness, to improve cell growth or combinations thereof.

The ECM cell suspension mixture may be centrifuged at approximately 125 Xg for approximately 10 minutes at approximately 4° C. Other centrifugation relative centrifugal forces, other times, and other temperatures may be utilized, and these parameters may be optimized, as a non-limiting example, for the type(s) of cell being used. As non-limiting examples, cell suspensions may be centrifuged at approximately 25 Xg to approximately 500 Xg, at approximately 50 Xg to approximately 275 Xg, at approximately 100 Xg to approximately 400 Xg, at approximately 75 Xg to approximately 250 Xg, at approximately 100 Xg to approximately 225 Xg, at approximately 105 Xg to approximately 215 Xg, at approximately 110 Xg to approximately 200 Xg, at approximately 115 Xg to approximately 175 Xg, at approximately 120 Xg to approximately 150 Xg, at approximately 125 Xg to approximately 400 Xg, at approximately 150 Xg, at approximately 135 Xg, at approximately 130 Xg, or at approximately 125 Xg, at approximately 120 Xg or at approximately 115 Xg. As non-limiting examples, cell suspensions may be centrifuged for approximately 2 minutes to 30 minutes, for approximately 4 minutes to 20 minutes, for approximately 5 minutes to 15 minutes, or for approximately 10 minutes. As non-limiting examples, cell suspensions may be centrifuged at approximately 1° C. to 20° C., at approximately 3° C. to 15° C., at approximately 4° C. to 8° C., at approximately 8° C., at approximately 7° C., at approximately 6° C. at approximately 5° C., at approximately 4° C. at approximately 3° C., at approximately 2° C., or at approximately 1° C. Centrifugation relative centrifugal forces and temperatures may be adjusted according, as a non-limiting example, to cell type(s). As a non-limiting example, larger cells may require larger centrifugal forces, longer times centrifuging, or combinations thereof, in order to ground the cells to the bottom of the plate. In embodiments, the centrifugation temperature may be chosen so that the ECM reagent may not solidify before the end of the centrifugation.

Cultures may be maintained at cell culture conditions known to be used or usable for the cell type(s). For example, cultures may be maintained in a humidified incubator at approximately 37° C. and approximately 5% CO₂. The culture may be monitored for approximately 7 to approximately 10 days, or for another desired period of time. Other culture times may be utilized, and the culture time may be optimized for the type(s) of cell being used. A non-limiting example of a suitable cell monitoring process is monitoring using an IncuCyte® live cell imaging and analysis platform. Other optical microscopy techniques and systems may be employed for monitoring spheroids. Optionally, scanning electron microscopy and/or transmission electron microscopy (TEM) may be employed.

Media may be changed and/or characterization and/or analysis and/or testing and/or challenging (such as, but not limited to, addition of T cells) may be performed on day approximately 3, approximately 4 or approximately 5, or at another desired time or times. Other time intervals may be utilized, and the time interval may be optimized for the type(s) of cell being used and/or for the type(s) of characterization and/or analysis and/or testing and/or challenging that may be performed.

In embodiments, the procedures may be performed in the above order. In embodiments, cells may be centrifuged before ECM is added to the cells. In embodiments, cells may be seeded before ECM is added to the cells. In embodiments, cells may be seeded, then the cells may be centrifuged, then ECM may be added to the cells. In embodiments, cells, with or without ECM, may be centrifuged at least two times.

As used herein, “new protocol”, “new method”, “improved method”, “Protocol A”, and/or plural tenses of these phrases, may refer to a method or methods of spheroid generation comprising some or all of the above procedures set forth in this section (New Methods of Spheroid Production) in the order set forth and/or comprising (i) centrifuging a cell suspension during the protocol before adding ECM, (ii) seeding a cell suspension during the protocol before adding ECM, or (iii) combinations of (i) and (ii).

Adjusted New Methods (Protocol B) of Spheroid Production

A spheroid or spheroids (e.g., “spheroid(s)”) in a certain volume of media may be desirable for characterizing, analyzing, challenging, otherwise testing, or a combination thereof, of at least one spheroid, which volume may be referred to herein as a “testing volume” or “V”. For spheroids formed in a testing volume (V) of media (including ECM), approximately one half volume (V_{1/2}) of media may commonly be changed with approximately one half of volume (V_{1/2}) of fresh media, or approximately one half of volume (V_{1/2}) of media may commonly be removed and a test composition and/or control in approximately one half volume (V_{1/2}) of media may commonly be added. In other words, media is commonly removed, and additional media, a test composition, and/or a control is added.

When removing media, however, spheroid(s) may be disrupted and/or change location in a cell culture well or other cell culture apparatus. In embodiments, it may be desirable to reduce or eliminate disruption and/or movement of spheroid(s). In embodiments, this may be accomplished by partially or fully avoiding removal of media. For example, cells may be seeded in a first volume that is a first fraction of a testing volume V that is desired for characterization, analysis, challenging, otherwise testing, or a combination thereof, of at least one spheroid; the cells may then be centrifuged; ECM may then be added to the cells in a second volume that is a second fraction of the testing volume V; and at least one spheroid may be allowed to form. In an embodiment, media, a control, a test composition, or combinations thereof may be added to the at least one spheroid (with ECM) in a volume that results in a combined volume of the testing volume V. A test composition may comprise, as non-limiting examples, engineered cells, such as but not limited to engineered T cells, drug(s), nutrients(s), or combinations thereof. A control may comprise, as non-limiting examples, media, non-engineered cells, differently engineered cells, different drug(s), different nutrient(s), different concentrations of a testing composition, or a combination thereof. Using such methods, removal of media from cell cultures may be fully or partially avoided. Using such methods, disruption and/or movement of spheroid(s) may be lessened or avoided.

For example, where a testing volume that is desired for characterization, analysis, challenging, otherwise testing, or a combination thereof, of at least one spheroid, is V, “V_{F1}” may be a volume that is a first fraction of the testing volume of V, and “V_{F2}” may be a volume that is a second fraction of the volume of V. In an embodiment, cells may be initially seeded in V_{F1} volume media, the cells may be centrifuged, then V_{F2} volume ECM reagent may be added, for a total volume of V_{F1} plus V_{F2} (“V_{F1}+V_{F2}”)(including ECM). V_{F1} may be the same as V_{F2}, or V_{F1} may be different from V_{F2}. As non-limiting examples, a fraction (F) may be 1/10, ⅛, ⅙, ⅕, ⅓, ⅔, or ¼ of a testing volume (V). In an embodiment, media and/or a control and/or a test composition, in a volume sufficient to a produce a testing volume V, may be added to at least one spheroid in a V_{F1} V_{F2} volume of media (including ECM). In an embodiment, for spheroid(s) in V_{F1} V_{F2} volume of media (including ECM), a third volume of media “V_{F3}”, where is V_{F3} is a fraction of V, may be added, or a test composition or control in a volume of V_{F3} may be added, or combinations thereof, with either or combinations thereof resulting in a testing volume of approximately V. In embodiments, V_{F3} may be approximately equal to V minus (V_{F1} plus V_{F2}), i.e., V_{F3} (V−(V_{F1} V_{F2})). In embodiments, V may be approximately equal to V_{F1}+V_{F2} V_{F3}; i.e., V−V_{F1}+V_{F2} V_{F3}. V_{F1}, V_{F2}, and V_{F3} may be the same, two may be the same as each other but different from the other, or they all may be different from one another.

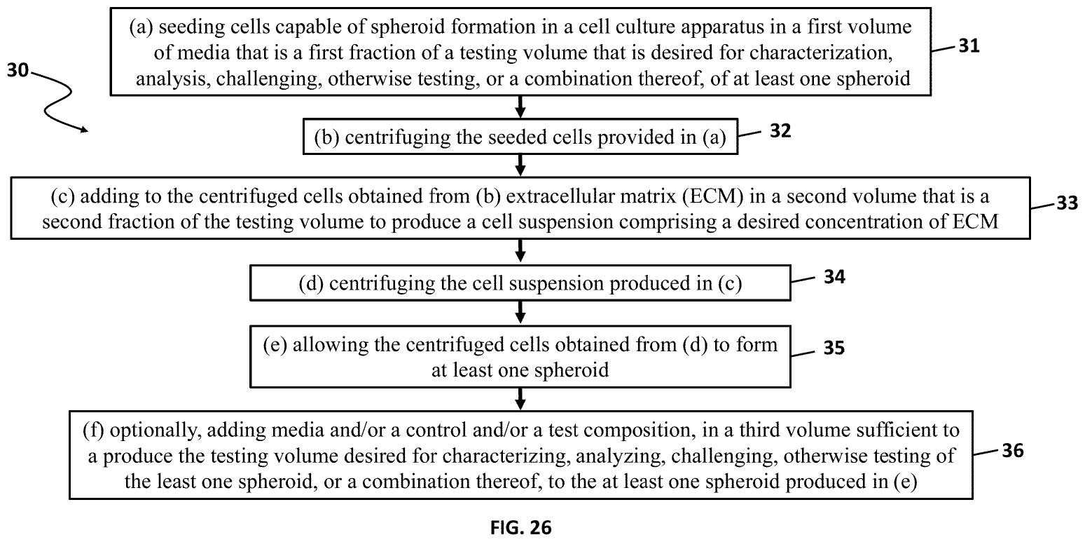

FIG. 26 shows an exemplary Protocol B of spheroid production according to an embodiment of the present disclosure. An exemplary Protocol B (30) may include (a) seeding cells capable of spheroid formation in a cell culture apparatus in a first volume of media that is a first fraction of a testing volume that is desired for characterization, analysis, challenging, otherwise testing, or a combination thereof, of at least one spheroid (31), (b) centrifuging the seeded cells provided in (a) (32), adding to the centrifuged cells obtained from (b) extracellular matrix (ECM) in a second volume that is a second fraction of the testing volume to produce a cell suspension comprising a desired concentration of ECM, (d) centrifuging the cell suspension produced in (c) (34), and (e) allowing the centrifuged cells obtained from (d) to form at least one spheroid (35), and optionally, (f) adding to the at least one spheroid produced in (e) media and/or a control and/or a test composition, in a volume sufficient to a produce the testing volume (36).

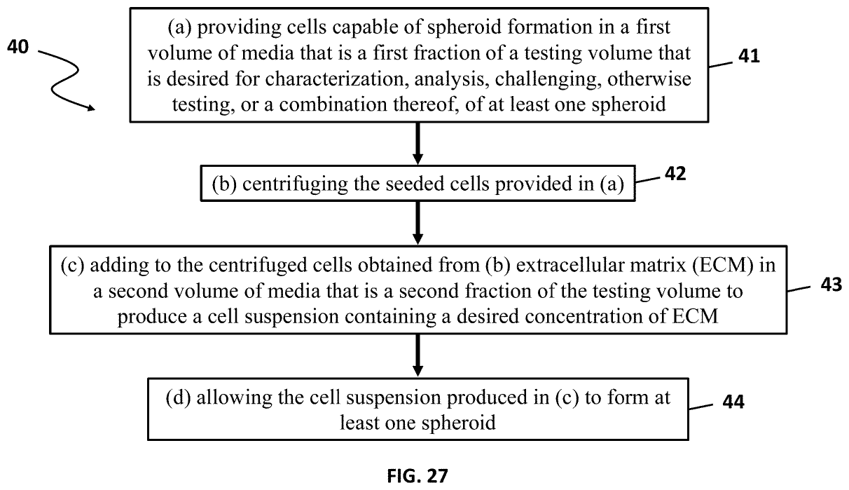

FIG. 27 shows an exemplary Protocol B of spheroid production according to another embodiment of the present disclosure. An exemplary Protocol B (40) may include (a) providing cells capable of spheroid formation in a cell culture apparatus in a first testing volume of media that is a first fraction of a volume that is desired for characterization, analysis, challenging, otherwise testing, or a combination thereof, of at least one spheroid (41), (b) centrifuging the seeded cells provided in (a) (42), (c) adding to the centrifuged cells obtained from (b) extracellular matrix (ECM) in a second volume of media that is a second fraction of the testing volume to produce a cell suspension containing a desired concentration of ECM (43), and (d) allowing the cell suspension produced in (c) to form at least one spheroid (44).

As a non-limiting example, where a certain volume is “V”, “V_{1/2}” may be a volume that is approximately one half of the volume of V, and “V_{1/4}” may be a volume that is approximately one quarter of the volume of V. As a non-limiting example, cells may be initially seeded in V_{1/4} (e.g., 50 μl) volume media, the cells may be centrifuged, then V_{1/4} volume ECM reagent may be added, for a combined volume of V_{1/2} (e.g., 100 μl) (including ECM). As another non-limiting example, cells may be initially seeded in V_{1/4} (e.g., 35 μl) volume media, the cells may be centrifuged, then V_{1/4} volume ECM reagent may be added, for a total volume of V_{1/2} (e.g., 70 μl) (including ECM). As another non-limiting example, where a certain volume is “V”, “V_{1/3}” may be a volume that is approximately one third of the volume of V, and “V_{2/3}” may be a volume that is approximately two thirds of the volume of V. As another non-limiting example, cells may be initially seeded in V_{1/3} (e.g., 50 μl) volume media, the cells may be centrifuged, then V_{1/3} volume ECM reagent may be added, for a combined volume of V_{2/3} (e.g., 100 μl) (including ECM). As another non-limiting example, cells may be initially seeded in V_{1/3} (e.g., 40 μl) volume media, the cells may be centrifuged, then V_{1/3} volume ECM reagent may be added, for a combined volume of V_{2/3} (e.g., 80 μl) (including ECM). As another non-limiting example, cells may be initially seeded in V_{F1} (e.g., 50 μl) volume media, the cells may be centrifuged, then V_{F2} (e.g., 60 μl) volume ECM reagent may be added, for a combined volume of V_{F1} V_{F2} (e.g., 110 μl) (including ECM). As a non-limiting example, where V may be approximately e.g., 2004, or 140 μL, for spheroid(s) in V_{1/2} (e.g., 1004, or 70 μL) volume of media (including ECM), approximately V−V_{1/2}=V_{1/2} (e.g., 100 μl, L or 70 μL) volume of fresh media may be added, or a test composition or control in approximately V−V_{1/2}=V_{1/2} volume of media may be added, with either resulting in a volume of approximately V (e.g., 2004, or 140 μL). As another non-limiting example, where V may be approximately, e.g., 1504, or 120 μL, for spheroid(s) in V_{2/3} (e.g., 1004, or 80 μL) volume of media (including ECM), approximately V−V_{2/3}=V_{1/3} (e.g., 504, or 40 μL) volume of fresh media may be added, or a test composition or control in approximately V−V_{2/3}=V_{1/3} volume of media may be added, with either resulting in a volume of approximately V (e.g., 150 μl, L or 120 μl, L). As another non-limiting example, where V may be approximately, e.g., 200 μL, for spheroid(s) in V_{F1}+V_{F2} (e.g., 110 μL) volume of media (including ECM), approximately V−(V_{F1}+V_{F2}) (e.g., 90 μL) volume of fresh media may be added, or a test composition or control in approximately V−(V_{F1}+V_{F2}) volume of media may be added, with either resulting in a volume of approximately V (e.g., 200 μL). Volumes given as examples (“e.g.”) are non-limiting examples.

In embodiments, an adjusted new protocol (Protocol B) may comprise other procedures, such as those described for a new protocol (Protocol A). In embodiments, the procedures may be performed in an order described for a new protocol (Protocol A). In embodiments, cells may be centrifuged before ECM is added to the cells. In embodiments, cells may be seeded before ECM is added to the cells. In embodiments, cells may be seeded, then the cells may be centrifuged, then ECM may be added to the cells. In embodiments, cells, with or without ECM, may be centrifuged at least two times during a protocol. Parameters for seeding, centrifuging, addition of ECM, and other procedures may vary, such as, but not limited to, as described with relation to a new protocol (Protocol A). Volume may vary, as non-limiting examples, according to working volume of a culture apparatus, according to desired volume for characterizing, analyzing, challenging, otherwise testing spheroid(s), or a combination thereof.

In embodiments, in an adjusted new protocol (Protocol B), cell suspension may be seeded before ECM is added. Cell suspension may be centrifuged before ECM is added. Cell suspension may be centrifuged after seeding and before adding ECM. Cell suspension may be centrifuged a first time during a protocol in a volume of V_{F}. Cell concentration before seeding in a volume of V_{F} may be adjusted to achieve a desired final concentration of cells in V (once other components in media are added). A desired final concentration may be a volume desired characterizing, analyzing, challenging, otherwise testing spheroid(s), or a combination thereof. A desired cell concentration may be, as a non-limiting example, approximately 1,000 cells per culture apparatus partition, such as, but not limited to, approximately 1,000 cells per well of a 96 well plate.

As used herein, “adjusted new protocol”, “adjusted new method”, “adjusted improved method”, “Protocol B”, and/or the plural tenses of these phrases may refer to a method or methods of spheroid generation comprising some or all of the above procedures and/or comprising (i) centrifuging a cell suspension during the protocol before adding ECM, (ii) seeding a cell suspension during the protocol before adding ECM in a volume of media that comprises a first fraction of a testing volume V that is desired for characterization, analysis, challenging, otherwise testing, or a combination thereof, of at least one spheroid, or (iii) combinations of (i) and (ii). In embodiments, using Protocol B, no media removal may be performed so that spheroid(s) may not be disrupted and/or change location in a cell culture well or other cell culture apparatus.

### Potential Exemplary Attributes of Spheroids or Cell Cultures Generated Using a New Protocol (Protocol A)

In embodiments, cells treated with a new protocol (Protocol A) described herein may form spheroid(s) and/or grow at rates similar to or approximately the same as cells treated with common protocols described herein. In embodiments, treatment of cells with a new protocol (Protocol A) may not negatively affect or decrease spheroid(s) formation rates and/or cell growth rates, as compared to cells treated with a common protocol described herein.

In embodiments, treatment of cells with a new protocol (Protocol A) may positively affect or may increase spheroid(s) formation rates and/or cell growth rates, as compared to cells treated with common protocols described herein. In embodiments, cultures of spheroid(s) generated using a new protocol (Protocol A) may comprise a combination of these attributes (positive attributes).

In embodiments, cells treated with a new protocol (Protocol A) may form spheroid(s) and/or grow at rates similar to or approximately the same as cells treated using (i) a protocol that does not comprise centrifugation of cells prior to adding ECM to the cells to produce a cell suspension comprising a desired concentration of extracellular matrix, (ii) a protocol that does not comprise seeding cells in a cell culture apparatus prior to adding ECM to the cells to produce a cell suspension comprising a desired concentration of extracellular matrix, or (iii) a protocol that comprises neither centrifugation of cells nor seeding cells in a cell culture apparatus prior to adding ECM to the cells to produce a cell suspension comprising a desired concentration of extracellular matrix.

In embodiments, treatment of cells with a new protocol (Protocol A) may not negatively affect or decrease spheroid(s) formation rates and/or cell growth rates, as compared to cells treated using (i) a protocol that does not comprise centrifugation of cells prior to adding ECM to the cells to produce a cell suspension comprising a desired concentration of extracellular matrix, (ii) a protocol that does not comprise seeding cells in a cell culture apparatus prior adding ECM to the cells to produce a cell suspension comprising a desired concentration of extracellular matrix, or (iii) a protocol that comprises neither centrifugation of cells nor seeding cells in a cell culture apparatus prior to adding ECM to the cells to produce a cell suspension comprising a desired concentration of extracellular matrix.

In embodiments, treatment of cells with a new protocol (Protocol A) may positively affect or may increase spheroid(s) formation rates and/or cell growth rates, as compared to cells treated (i) a protocol that does not comprise centrifugation of cells prior to adding ECM to the cells to produce a cell suspension comprising a desired concentration of extracellular matrix, (ii) a protocol that does not comprise seeding cells in a cell culture apparatus prior adding ECM to the cells to produce a cell suspension comprising a desired concentration of extracellular matrix, or (iii) a protocol that comprises neither centrifugation of cells nor seeding cells in a cell culture apparatus prior to adding ECM to the cells to produce a cell suspension comprising a desired concentration of extracellular matrix. In embodiments, cultures of spheroid(s) generated using a new protocol (Protocol A) may comprise a combination of these attributes (positive attributes).

The phrases “a protocol that does not comprise centrifugation of cells prior to adding ECM to the cells to produce a cell suspension comprising a desired concentration of extracellular matrix”, “a protocol that does not comprise seeding cells in a cell culture apparatus prior adding ECM to the cells to produce a cell suspension comprising a desired concentration of extracellular matrix”, and “a protocol that comprises neither centrifugation of cells nor seeding cells in a cell culture apparatus prior to adding ECM to the cells to produce a cell suspension comprising a desired concentration of extracellular matrix” refer to actions performed as a part of a spheroid generation protocol, and are not intended to refer to actions that may or may not have been performed on the cells prior to the commencement of the spheroid generation protocol. For example, these phrases contemplate that the cells, population of cells, or cell line used in the spheroid generation protocol may have been centrifuged, seeded, or both at one or multiple times before commencement of the spheroid generation protocol.

In embodiments, cultures of spheroid(s) generated using a new protocol (Protocol A) may comprise spheroid(s) having a more uniform morphology, spheroid(s) having a more compact structure, spheroid(s) having a lower level of shape eccentricity (a higher level of circularity or other shape uniformity), larger spheroids, a higher density (degree of compactness), or a combination thereof, as compared to cultures generated using common protocols described herein. In embodiments, cultures of spheroid(s) generated using a new protocol (Protocol A) may comprise a lower percentage of non-spheroid cellular aggregates and/or spheroids lacking uniform size, as compared to cultures generated using common protocols described herein. In embodiments, in cultures generated using a new protocol (Protocol A), at least one spheroid may be generated per culture well or other culture apparatus partition, cellular debris, spheroids lacking uniform size, and/or non-spheroid cellular aggregates may be absent or may be present in lower amounts or numbers as compared to cultures generated using common protocols described herein, or a combination thereof.

In embodiments, in cultures generated using a new protocol (Protocol A), only one spheroid may be generated per culture well or other culture apparatus partition, cellular debris, spheroids lacking uniform size, and/or non-spheroid cellular aggregates may be absent or may be present in lower amounts or numbers as compared to cultures generated using common protocols described herein, or a combination thereof. In embodiments, in cultures generated using a new protocol (Protocol A), the spheroid(s) may be generated more centrally located in the culture well or other culture apparatus partition or may be larger, as compared to cultures generated using common protocols described herein. In embodiments, cultures of spheroid(s) generated using a new protocol (Protocol A) may comprise a combination of these attributes (positive attributes).

In embodiments, cultures of spheroid(s) generated using a new protocol (Protocol A) may comprise spheroid(s) having a more uniform morphology, spheroid(s) having a more compact structure, spheroid(s) having a lower level of shape eccentricity (a higher level of circularity or other shape uniformity), larger spheroids, a higher density (degree of compactness), or a combination thereof, as compared to cultures generated using (i) a protocol that does not comprise centrifugation of cells prior to adding ECM to the cells to produce a cell suspension comprising a desired concentration of extracellular matrix, (ii) a protocol that does not comprise seeding the cells in a cell culture apparatus adding ECM to cells to produce a cell suspension comprising a desired concentration of extracellular matrix, or (iii) a protocol that comprises neither centrifugation of cells nor seeding cells in a cell culture apparatus prior to adding ECM to the cells to produce a cell suspension comprising a desired concentration of extracellular matrix.

In embodiments, cultures of spheroid(s) generated using a new protocol (Protocol A) may comprise a lower percentage of non-spheroid cellular aggregates and/or spheroids lacking uniform size, as compared to cultures generated using (i) a protocol that does not comprise centrifugation of cells prior to adding ECM to the cells to produce a cell suspension comprising a desired concentration of extracellular matrix, (ii) a protocol that does not comprise seeding cells in a cell culture apparatus prior adding ECM to cells to produce a cell suspension comprising a desired concentration of extracellular matrix, or (iii) a protocol that comprises neither centrifugation of cells nor seeding the cells in a cell culture apparatus prior to adding ECM to the cells to produce a cell suspension comprising a desired concentration of extracellular matrix. In embodiments, in cultures generated using a new protocol (Protocol A), at least one spheroid may be generated per culture well or other culture apparatus partition, cellular debris, spheroids lacking uniform size, and/or non-spheroid cellular aggregates may be absent or may be present in lower amounts or numbers, or a combination thereof, as compared to cultures generated using (i) a protocol that does not comprise centrifugation of cells prior to adding ECM to the cells to produce a cell suspension comprising a desired concentration of extracellular matrix, (ii) a protocol that does not comprise seeding cells in a cell culture apparatus prior adding ECM to the cells to produce a cell suspension comprising a desired concentration of extracellular matrix, or (iii) a protocol that comprises neither centrifugation of cells nor seeding cells in a cell culture apparatus prior to adding ECM to the cells to produce a cell suspension comprising a desired concentration of extracellular matrix.

In embodiments, in cultures generated using a new protocol (Protocol A), only one spheroid may be generated per culture well or other culture apparatus partition, cellular debris, spheroids lacking uniform size, and/or non-spheroid cellular aggregates may be absent or may be present in lower amounts or numbers, or a combination thereof, as compared to cultures generated using (i) a protocol that does not comprise centrifugation of cells prior to adding ECM to the cells to produce a cell suspension comprising a desired concentration of extracellular matrix, (ii) a protocol that does not comprise seeding cells in a cell culture apparatus prior adding ECM to the cells to produce a cell suspension comprising a desired concentration of extracellular matrix, or (iii) a protocol that comprises neither centrifugation of cells nor seeding cells in a cell culture apparatus prior to adding ECM to the cells to produce a cell suspension comprising a desired concentration of extracellular matrix.

In embodiments, in cultures generated using a new protocol (Protocol A), the spheroid(s) may be generated more centrally located in the culture well or other culture apparatus partition, as compared to cultures generated using (i) a protocol that does not comprise centrifugation of cells prior to adding ECM to the cells to produce a cell suspension comprising a desired concentration of extracellular matrix, (ii) a protocol that does not comprise seeding cells in a cell culture apparatus prior adding ECM to the cells to produce a cell suspension comprising a desired concentration of extracellular matrix, or (iii) a protocol that comprises neither centrifugation of cells nor seeding cells in a cell culture apparatus prior to adding ECM to the cells to produce a cell suspension comprising a desired concentration of extracellular matrix.

In embodiments, cultures of spheroid(s) generated using a new protocol (Protocol A) may comprise a combination of these attributes (positive attributes). The phrases “a protocol that does not comprise centrifugation of cells prior to adding ECM to the cells to produce a cell suspension comprising a desired concentration of extracellular matrix”, “a protocol that does not comprise seeding cells in a cell culture apparatus prior adding ECM to the cells to produce a cell suspension comprising a desired concentration of extracellular matrix”, and “a protocol that comprises neither centrifugation of cells nor seeding cells in a cell culture apparatus prior to adding ECM to the cells to produce a cell suspension comprising a desired concentration of extracellular matrix” refer to actions performed as a part of a spheroid generation protocol, and are not intended to refer to actions that may or may not have been performed on the cells prior to the commencement of the spheroid generation protocol. For example, these phrases contemplate that the cells, population of cells, or cell line used in the spheroid generation protocol may have been centrifuged, seeded, or both at one or multiple times before commencement of the spheroid generation protocol.

In embodiments, in cultures generated using a new protocol (Protocol A), at least approximately 50% or more of the culture wells or other culture apparatus partitions may comprise a culture having at least one positive attribute. In embodiments, in cultures generated using a new protocol (Protocol A), at least approximately 55% or more of the culture wells or other culture apparatus partitions may comprise a culture having at least one positive attribute. In embodiments, in cultures generated using a new protocol (Protocol A), at least approximately 60% or more of the culture wells or other culture apparatus partitions may comprise a culture having at least one positive attribute. In embodiments, in cultures generated using a new protocol (Protocol A), at least approximately 65% or more of the culture wells or other culture apparatus partitions may comprise a culture having at least one positive attribute. In embodiments, in cultures generated using a new protocol (Protocol A), at least approximately 70% or more of the culture wells or other culture apparatus partitions may comprise a culture having at least one positive attribute. In embodiments, in cultures generated using a new protocol (Protocol A), at least approximately 50% or more, at least approximately 55% or more, at least approximately 60% or more, at least approximately 65% or more, at least approximately 70% or more, at least approximately 75% or more, at least approximately 80% or more at least approximately 85% or more, at least approximately 90% or more, at least approximately 95% or more, or approximately 100% of the culture wells or other culture apparatus partitions may comprise a culture having at least one positive attribute.

In embodiments, cultures of spheroid(s) generated using a new protocol (Protocol A) may comprise spheroid(s) having a circularity of approximately 0.1 to approximately 1.0, a circularity of approximately 0.1 to approximately 1.0, a circularity of approximately 0.2 to approximately 1.0, a circularity of approximately 0.4 to approximately 1.0, a circularity of approximately 0.5 to approximately 1.0, a circularity of approximately 0.6 to approximately 1.0, a circularity of approximately 0.7 to approximately 0.95, a circularity of approximately 0.7 to approximately 0.9, a circularity of approximately 0.8 to approximately 0.85, a circularity of at least approximately 0.3, a circularity of at least approximately 0.4, a circularity of at least approximately 0.45, a circularity of at least approximately 0.5, a circularity of at least approximately 0.55, a circularity of at least approximately 0.6, a circularity of at least approximately 0.65, a circularity of at least approximately 0.7, a circularity of at least approximately 0.75, a circularity of at least approximately 0.8, a circularity of at least approximately 0.85, a circularity of at least approximately 0.9, a circularity of at least approximately 0.95, a circularity of at least approximately 0.97, a circularity of at least approximately 0.98, a circularity of at least approximately 0.99, or a circularity of approximately 1.

In embodiments, cultures of spheroid(s) generated using a new protocol (Protocol A) may comprise spheroid(s) having an eccentricity of approximately 0.0 to approximately 0.7, an eccentricity of approximately 0.6 to approximately 0.75, an eccentricity of approximately 0.5 to approximately 0.95, an eccentricity of approximately 0.5 to approximately 0.9, an eccentricity of approximately 0.4 to approximately 0.85, an eccentricity of approximately 0.3 to approximately 0.7, an eccentricity of approximately 0.2 to approximately 0.65, an eccentricity of approximately 0.1 to approximately 0.5, an eccentricity of approximately 0.1 to approximately 0.4, an eccentricity of approximately 0.1 to approximately 0.3, an eccentricity of approximately 0.1 to approximately 0.2, an eccentricity of approximately 0.1, an eccentricity of approximately 0.05, an eccentricity of approximately 0.02, an eccentricity of approximately 0.01, an eccentricity of approximately 0, an eccentricity of approximately 0.7 or less, an eccentricity of approximately 0.65 or less, an eccentricity of approximately 0.6 or less, an eccentricity of approximately 0.55 or less, an eccentricity of approximately 0.5 or less, an eccentricity of approximately 0.45 or less, an eccentricity of approximately 0.4 or less, an eccentricity of approximately 0.35 or less, an eccentricity of approximately 0.3 or less, an eccentricity of approximately 0.25 or less, an eccentricity of approximately 0.2 or less, an eccentricity of approximately 0.15 or less, an eccentricity of approximately 0.1 or less, an eccentricity of approximately 0.05 or less, an eccentricity of approximately 0.02 or less, an eccentricity of approximately 0.01 or less, or an eccentricity of approximately 0.

In embodiments, cultures of spheroids generated using a new protocol (Protocol A) may comprise at least two spheroids having a difference from one another in eccentricity, in circularity, in compactness, in size, or a combination thereof, of less than about 1%, less than about 5%, less than about 15%, less than about 20%, less than about 25%, less than about 30%, less than about 35%, less than about 40%, less than about 45%, less than about 50%, less than about 55%, less than about 60%, less than about 70%, or less than about 75%. “Cultures of spheroids” may refer to multiple spheroids within a multiple partitions of a cell culture apparatus or apparatuses (e.g., in a multiple wells of a plate or plates), or to a single spheroid or multiple spheroids within a single partition of a cell culture apparatus (e.g., in a single well of a plate). Spheroids produced across one iteration of a protocol may be compared. Spheroids produced across multiple iterations of a protocol may be compared. Spheroids of the same cell type(s) may be compared. Spheroids of different cell type(s) may be compared.

In embodiments, spheroids generated with the improved methods described herein result in individual cell culture apparatus partitions with 95% or less, 90% or less, 85% or less, 80% or less, 75% or less, 60% or less, 50% or less, 40% or less, 30% or less, or 25% or less non-spheroid cellular aggregates as compared to spheroids generated in the same sized tissue culture partition, generated using the same seeding concentration of cells, or generated using both, using a common protocol described herein. In another aspect, spheroids generated with the improved methods described herein result in wells with 95% or less, 90% or less, 85% or less, 80% or less, 75% or less, 60% or less, 50% or less, 40% or less, 30% or less, or 25% or less cellular debris as compared to spheroids generated in the same sized well using a common protocol described herein.

In embodiments, spheroids generated with the improved methods described herein result in individual cell culture apparatus partitions with approximately 95% or less, approximately 90% or less, approximately 85% or less, approximately 80% or less, approximately 75% or less, approximately 60% or less, approximately 50% or less, approximately 40% or less, approximately 30% or less, or approximately 25% or less non-spheroid cellular aggregates as compared to spheroids generated in the same sized tissue culture partition, generated using the same seeding concentration of cells, or generated using both, as compared to cultures generated using (i) a protocol that does not comprise centrifugation of cells prior to adding ECM to the cells to produce a cell suspension comprising a desired concentration of extracellular matrix, (ii) a protocol that does not comprise seeding cells in a cell culture apparatus prior adding ECM to the cells to produce a cell suspension comprising a desired concentration of extracellular matrix, or (iii) a protocol that comprises neither centrifugation of cells nor seeding cells in a cell culture apparatus prior to adding ECM to the cells to produce a cell suspension comprising a desired concentration of extracellular matrix. Percentage cellular debris may be calculated, as non-limiting examples, by mass, volume, or diameter.

In embodiments, spheroids generated with the improved methods described herein result in individual cell culture apparatus partitions with approximately 95% or less, approximately 90% or less, approximately 85% or less, approximately 80% or less, approximately 75% or less, approximately 60% or less, approximately 50% or less, approximately 40% or less, approximately 30% or less, or approximately 25% or less cellular debris as compared to spheroids generated in the same sized tissue culture partition, generated using the same seeding concentration of cells, or generated using both, as compared to cultures generated using (i) a protocol that does not comprise centrifugation of cells prior to adding ECM to the cells to produce a cell suspension comprising a desired concentration of extracellular matrix, (ii) a protocol that does not comprise seeding cells in a cell culture apparatus prior adding ECM to the cells to produce a cell suspension comprising a desired concentration of extracellular matrix, or (iii) a protocol that comprises neither centrifugation of cells nor seeding cells in a cell culture apparatus prior to adding ECM to the cells to produce a cell suspension comprising a desired concentration of extracellular matrix. Percentage cellular debris may be calculated, as non-limiting examples, by mass, volume, or diameter.

In embodiments, the improved methods described herein may generate a single spheroid (per well) with a size range of about 200 μm to about 750 μm, about 250 μm to about 700 μm, about 300 μm to about 650 μm, about 400 μm to about 600 μm, about 450 μm to about 550 μm, or about 500 μm in a well of approximately 75 μL to approximately 200 μL working volume 200 μl working volume at about 24 hours to about 120 hours, about 36 hours to about 96 hours, about 48 hours to about 84 hours, or about 96 hours after seeding. In embodiments, the improved methods described herein may generate a single spheroid (per well) with a size of about 450 μm to about 550 μm along at least one axis, within about 84 to about 108 hours after cell seeding. In embodiments, the improved methods described herein may generate a single spheroid (per well) with a size of about 400 μm to about 600 μm along at least one axis or dimension, within about 96 hours after cell seeding. In embodiments, the improved methods described herein may generate a single spheroid (per well) with a size of about 450 μm to about 550 μm along at least one axis or dimension, within about 96 hours after cell seeding. In embodiments, the improved methods described herein may generate a single spheroid (per well) with a size of about 500 μm along at least one axis or dimension, within about 96 hours after cell seeding.

In an aspect, spheroids generated with a new protocol (Protocol A) described herein may result in two or fewer, three or fewer, four or fewer, five or fewer, 10 or fewer, 20 or fewer, or 30 or fewer non-spheroid cellular aggregates/cellular debris-like particles per approximately 1×10² to approximately 20×10⁶ cells seeded, approximately 1×10³ to approximately 20×10⁵ cells seeded, approximately 2×10³ to approximately 10×10⁵ cells seeded, approximately 1×10⁴ to approximately 10×10⁶ cells seeded, approximately 1.5×10⁴ to approximately 10×10⁶ cells seeded, approximately 5×10⁴ to approximately 5×10⁶ cells seeded, approximately 1×10⁵ to approximately 5×10⁵ cells seeded, approximately 1×10⁴ to approximately 10×10⁴ cells seeded, approximately 1×10⁴ to approximately 10×10⁴ cells seeded, approximately 1.5×10⁴ to approximately 9×10⁴ cells seeded, approximately 1.5×10⁴ to approximately 5×10⁴ cells seeded, approximately 2×10⁴ to approximately 4×10⁴ cells seeded, approximately 2×10⁴ to approximately 3×10⁴ cells seeded, approximately 1×10² cells seeded, approximately 5×10² cells seeded, approximately 1×10³ cells seeded, approximately 2×10³ cells seeded, approximately 5×10³ cells seeded, approximately 7×10³ cells seeded, approximately 1×10⁴ cells seeded, approximately 2×10⁴ cells seeded, approximately 3×10⁴ cells seeded, approximately 5×10⁴ cells seeded, approximately 7×10⁴ cells seeded.

In an embodiment, spheroid(s) generated using a new protocol (Protocol A) may more accurately mimic in vivo conditions, such as, but not limited to, cell-cell interactions, oxygen gradient(s), and nutrient gradient(s), as compared to spheroid(s) generated using a common protocol. In an embodiment, spheroid(s) generated using a new protocol (Protocol A) may more accurately mimic in vivo conditions, such as, but not limited to, cell-cell interactions, oxygen gradient(s), and nutrient gradient(s), as compared to cultures generated using (i) a protocol that does not comprise centrifugation of cells prior to adding ECM to the cells to produce a cell suspension comprising a desired concentration of extracellular matrix, (ii) a protocol that does not comprise seeding cells in a cell culture apparatus prior adding ECM to the cells to produce a cell suspension comprising a desired concentration of extracellular matrix, or (iii) a protocol that comprises neither centrifugation of cells nor seeding cells in a cell culture apparatus prior to adding ECM to the cells to produce a cell suspension comprising a desired concentration of extracellular matrix. In an embodiment, spheroid(s) generated using a new protocol (Protocol A) and having a size of approximately 400 μm to approximately 600 μm along at least one axis may more accurately mimic in vivo conditions, such as, but not limited to, cell-cell interactions, oxygen gradient(s), and nutrient gradient(s), as compared to spheroid(s) generated using a common protocol. In an embodiment, spheroid(s) generated using a new protocol (Protocol A) and having a size of approximately 400 μm to approximately 600 μm along at least one axis may more accurately mimic in vivo conditions, such as, but not limited to, cell-cell interactions, oxygen gradient(s), and nutrient gradient(s), as compared to cultures generated using (i) a protocol that does not comprise centrifugation of cells prior to adding ECM to the cells to produce a cell suspension comprising a desired concentration of extracellular matrix, (ii) a protocol that does not comprise seeding cells in a cell culture apparatus prior adding ECM to the cells to produce a cell suspension comprising a desired concentration of extracellular matrix, or (iii) a protocol that comprises neither centrifugation of cells nor seeding cells in a cell culture apparatus prior to adding ECM to the cells to produce a cell suspension comprising a desired concentration of extracellular matrix. In an embodiment, spheroid(s) generated using a new protocol (Protocol A) and having a size of approximately 500 μm along at least one axis may more accurately mimic in vivo conditions, such as, but not limited to, cell-cell interactions, oxygen gradient(s), and nutrient gradient(s), as compared to spheroid(s) generated using a common protocol. In an embodiment, spheroid(s) generated using a new protocol (Protocol A) and having a size of approximately 500 μm along at least one axis may more accurately mimic in vivo conditions, such as, but not limited to, cell-cell interactions, oxygen gradient(s), and nutrient gradient(s), as compared to cultures generated using (i) a protocol that does not comprise centrifugation of cells prior to adding ECM to the cells to produce a cell suspension comprising a desired concentration of extracellular matrix, (ii) a protocol that does not comprise seeding cells in a cell culture apparatus prior adding ECM to the cells to produce a cell suspension comprising a desired concentration of extracellular matrix, or (iii) a protocol that comprises neither centrifugation of cells nor seeding cells in a cell culture apparatus prior to adding ECM to the cells to produce a cell suspension comprising a desired concentration of extracellular matrix.

In embodiments, spheroids generated using a new protocol (Protocol A), cultures generated using a new protocol (Protocol A), or combinations thereof, may have any combination of these positive attributes, attributes, or combinations thereof.

### Potential Exemplary Attributes of Spheroids or Cell Cultures Generated Using an Adjusted New Protocol (Protocol B)

In embodiments, spheroids or cell cultures generated using an adjusted new protocol (Protocol B) may have one or more of the same or similar positive attributes, attributes, or combinations thereof, as spheroids or cell cultures generated using a new protocol (Protocol A), such as those positive attributes and attributes set forth in the above section entitled “Potential Exemplary Attributes of Spheroids or Cell Cultures Generated Using a New Protocol”.

### Potential Exemplary Uses of Spheroids or Cell Cultures Generated Using a New Protocol (Protocol A) or an Adjusted New Protocol (Protocol B)

In embodiments, the spheroid(s) generated using a new protocol (Protocol A) or adjusted new protocol (Protocol B) is characterized, analyzed, challenged, and/or otherwise tested. Characterization, analysis, challenging, and/or other testing may comprise exposing the spheroid(s) to at least one drug, potential or putative drug, nutrient, cell, engineered cell, type of radiation, or environmental condition. A “drug” may be a chemical substance that, when administered to a cell or cells, produces a biological effect. A “drug” may be an immune suppressor, non-limiting examples of which include TGF-beta, adenosine, IL-4, IL-10, and lactate. A non-limiting example of an engineered cell may be an engineered T cell, such as a T cell expressing an engineered T cell receptor (TCR), a chimeric antigen receptor (CAR), or another engineered receptor or component.

A T-cell may express exogenous TCRs and antigen binding proteins described in U.S. Patent Application Publication No. 2017/0267738; U.S. Patent Application Publication No. 2017/0312350; U.S. Patent Application Publication No. 2018/0051080; U.S. Patent Application Publication No. 2018/0164315; U.S. Patent Application Publication No. 2018/0161396; U.S. Patent Application Publication No. 2018/0162922; U.S. Patent Application Publication No. 2018/0273602; U.S. Patent Application Publication No. 2019/0016801; U.S. Patent Application Publication No. 2019/0002556; U.S. Patent Application Publication No. 2019/0135914; U.S. Pat. Nos. 10,538,573; 10,626,160; U.S. Patent Application Publication No. 2019/0321478; U.S. Patent Application Publication No. 2019/0256572; U.S. Pat. Nos. 10,550,182; 10,526,407; U.S. Patent Application Publication No. 2019/0284276; U.S. Patent Application Publication No. 2019/0016802; U.S. Patent Application Publication No. 2019/0016803; U.S. Patent Application Publication No. 2019/0016804; U.S. Pat. No. 10,583,573; U.S. Patent Application Publication No. 2020/0339652; U.S. Pat. Nos. 10,537,624; 10,596,242; U.S. Patent Application Publication No. 2020/0188497; U.S. Pat. No. 10,800,845; U.S. Patent Application Publication No. 2020/0385468; U.S. Pat. Nos. 10,527,623; 10,725,044; U.S. Patent Application Publication No. 2020/0249233; U.S. Pat. No. 10,702,609; U.S. Patent Application Publication No. 2020/0254106; U.S. Pat. No. 10,800,832; U.S. Patent Application Publication No. 2020/0123221; U.S. Pat. Nos. 10,590,194; 10,723,796; U.S. Patent Application Publication No. 2020/0140540; U.S. Pat. No. 10,618,956; U.S. Patent Application Publication No. 2020/0207849; U.S. Patent Application Publication No. 2020/0088726; and U.S. Patent Application Publication No. 2020/0384028; the contents of each of these publications and sequence listings described therein are herein incorporated by reference in their entireties.

The T-cell may be a αβ T cell, γδ T cell, natural killer T cell. Natural killer cell. In an embodiment, TCRs described herein are single-chain TCRs or soluble TCRs.

In an embodiment, a T-cell may co-express exogenous TCR, antigen binding protein, or both, with exogenous CD8 polypeptides, e.g., CD8αβ heterodimer, CD8α homodimer, and modified CD8α homodimer, in which CD8α stalk region is replaced with CD8β stalk region as described in Wong et al. “Stalk region of beta-chain enhances the coreceptor function of CD8.” J Immunol. 2003 Jul. 15; 171(2):867-74; the content of which is herein incorporated by reference by its entirety.

Live-cell imaging and analysis platforms, e.g., Incucyte® Live-Cell Analysis System, that can enable quantification of cell behavior over time (from hours to weeks) by automatically gathering and analyzing images around the clock may be used to monitor, characterize, analyze, challenge, and/or test the effect on spheroids induced by agents, which may include but not limited to organic compounds, inorganic compounds, viruses, prokaryotic cells, eukaryotic cells, naturally existing cells, and engineered cells.

As used herein, the term “reporter gene” may refer to gene coding for a protein that is susceptible to quantitative analysis when expressed and/or quantitative analysis on cells that express reporter genes. Any reporter proteins known so far may be applicable for the present disclosure, including but not limited to genes for Renilla luciferase, green fluorescent protein, firefly luciferase, red fluorescence protein, and secreted alkaline phosphatase (SeAP). It is preferred that more than one reporter gene is selected for the present disclosure from the group consisting of Renilla luciferase, red fluorescence protein, and green fluorescent protein.

In some embodiments, spheroids may inducibly or constitutively express exogenously transduced reporter genes. The effects on spheroids induced by agents described herein may be determined by methods including, but not limited to, measuring the size of the fluorescence area of spheroids, measuring the fluorescence intensity of spheroids, and/or measuring the eccentricity of spheroids.

In some embodiments, when the size of the at least one spheroid is greater, the fluorescence intensity of the at least one spheroid is greater, and/or the eccentricity of the at least one spheroid is lesser before the exposing spheroids to agents described herein compared to after the exposing spheroids to agents, these results may indicate that agents may possess a cytotoxic activity against spheroids. In some embodiments, when the size, the fluorescence intensity, and/or the eccentricity of the at least one spheroid before the exposing spheroids to agents described herein are similar to that after the exposing spheroids to agents, these results may indicate that agents may not possess cytotoxic activity against spheroids. In some embodiments, when the size of the at least one spheroid is lesser, the fluorescence intensity of the at least one spheroid is lesser, and/or the eccentricity of the at least one spheroid is greater before the exposing spheroids to agents described herein compared to that after the exposing spheroids to agents, these results may indicate that agents may possess a growth promoting activity on spheroids.

In some embodiments, agents may include T cell expressing exogenous TCR and exogenous CD8. Exogenous CD8 may include CD8αβ heterodimer or CD8α homodimer. T cell may include CD8+ T cell and/or CD4+ T cells.

In some embodiments, the effect of T cells or other effector cells on spheroids may be assessed. The ratio of effector to target cells may be any ratio of interest.

In some embodiments, methods described herein can be used to screen chemical libraries that are generated in the laboratory by those skilled in the art or available from many other sources. Some examples of sources for chemical libraries may comprise the National Cancer Institute (NCI) small molecule sample collection that has been screened in the NCI panel of 60 human tumor cell lines. In some embodiments, the NCI panel of 60 human tumor cell lines may be prepared by the method of the present disclosure to generate spheroids and to test the activity of agents on the resulting spheroids.

3D cell cultures may be useful for in vitro safety studies. Primary human cells may be used to form 3D spheroids in accordance with some embodiments of the present disclosure.

In some embodiments, primary human cells used for generating 3D spheroids may include human normal cells differentiated from induced pluripotent stem cell (iPSC)-derived or isolated from normal human adult tissues or combinations thereof.

In an embodiment, the characterizing, the analyzing, the challenging, or the otherwise testing may comprise: the at least one spheroid that may be or may be not stained with a fluorescent probe, and measuring the fluorescence intensity of the respective samples.

In an embodiment, the fluorescent probe may include a cell-permeable dye containing a chloromethyl or bromomethyl group that reacts with thiol groups, utilizing a glutathione S-transferase-mediated reaction leading to cell-impermeant reaction products.

As used herein, the term “fluorescent probe” may refer to any fluorescent dye that is susceptible to quantitative analysis when retained in a cell. Any fluorescent dyes known so far may be applicable for the present disclosure including, but not limited to, cell-permeable dyes containing a chloromethyl or bromomethyl group that reacts with thiol groups, utilizing a glutathione S-transferase-mediated reaction leading to cell-impermeant reaction products.

In some embodiments, cells may be stained in 2D preculture before the generation of spheroids or cells may be stained after spheroid formation using fluorescent probes. The effects on spheroids induced by agents described herein may be determined by methods including, but not limited to, measuring the size of the fluorescence area of spheroids, measuring the fluorescence intensity of spheroids, and/or measuring the eccentricity of spheroids.

The term “primary tumor sample” as used herein refers to a sample containing tumor material obtained from a subject having cancer. The term encompasses tumor tissue samples, for example, tissue obtained by surgical resection and tissue obtained by biopsy, such as for example, a core biopsy or a fine needle biopsy. The term also encompasses patient derived xenograft (PDX). Patient derived xenografts may be generated when cancerous tissue from a patient's primary tumor is implanted directly into an immunodeficient mouse (see, for example, Morton C L, Houghton P J (2007). “Establishment of human tumor xenografts in immunodeficient mice”. Nature Protocols 2 (2): 247-50; Siolas D, Hannon G J (September 2013). “Patient-derived tumor xenografts: transforming clinical samples into mouse models”. Cancer Research 73 (17): 5315-9). PDX can mirror patients' histopathological and genetic profiles. It has improved predictive power as preclinical cancer models, and enables the true individualized therapy and discovery of predictive biomarkers.

In some embodiments, the subject may be a human. In some embodiments, the subject may be a non-human mammal or a non-human vertebrate. In some embodiments, the subject may be laboratory animal, a mouse, a rat, a rodent, a farm animal, a pig, a cattle, a horse, a goat, a sheep, a companion animal, a dog a cat, or a guinea pig.

Tumor cell spheroids can be prepared by methods of the present disclosure.

In some embodiments, the primary tumor sample may be collected in a serum-supplemented medium, for example but not limited to, RPMI medium supplemented with 10% fetal bovine serum. The sample may be then minced, i.e., cut or chopped into tiny pieces. In some embodiments, the sample may be minced on ice. In some embodiments, the minced primary tumor sample may contain tumor pieces in the size of about 3 mm, 2.5 mm, 2.0 mm, 1.5 mm, 1.0 mm, 0.5, or 0.25 mm.

In some embodiments, the primary tumor sample may be not frozen and thawed.

In some embodiments, minced primary tumor sample may be frozen in a medium supplemented with serum and thawed prior to treating with the composition containing enzymes. In some embodiments, the minced primary tumor sample may be frozen for at least 6 hours 12 hours, 24 hours, 2 days, 1 week or one month. In some embodiments, the minced primary tumor sample may be frozen at −80° C. In some embodiments, the minced primary tumor sample may be frozen in liquid nitrogen. In some embodiments, the minced primary tumor sample may be frozen in a medium supplemented with serum. In some embodiments, the minced primary tumor sample may be frozen in a mixture containing serum and solvent such as Dimethyl sulfoxide (DMSO). In some embodiments, the minced primary tumor sample may be frozen in a mixture containing fetal bovine serum and Dimethyl sulfoxide (DMSO).

In some embodiments, the frozen minced primary tumor sample may be thawed, i.e., defrosted, before treating the sample with a composition comprising an enzyme. In some embodiments, the minced primary tumor sample may be thawed in a water bath kept at about 37° C. (e.g., 35° C., 36° C., 37° C., 38° C., or 39° C.). In some embodiments, the minced primary tumor sample may be thawed at room temperature.

The minced primary tumor sample may be treated with an enzyme mix to digest the tumor samples. In some embodiments, the composition containing an enzyme may include collagenase. In some embodiments, the composition containing an enzyme may include a serum-supplemented culture medium, insulin, one or more corticosteroids, one or more antibiotics, collagenase and optionally one or more growth factors. Serum-supplemented culture media, corticosteroids, antibiotics, and growth factors are well-known in the art. In some embodiments, the composition containing an enzyme may include DMEM or RPMI, fetal bovine serum, insulin, epidermal growth factor, hydrocortisone, Penicillin and/or Streptomycin, and collagenase. In some embodiments, the composition containing an enzyme may further include a buffering agent such as 4-(2-hydroxyethyl)-1-piperazineethanesulfonic acid (HEPES).

“Treating the minced primary tumor sample with a composition containing an enzyme” may include incubating the minced tumor samples with the enzyme composition for at least 1 hour. In some embodiments, the minced tumor samples may be incubated with the enzyme mix for at least 2 hours, at least 4 hours, at least 6 hours, at least 8 hours, at least 10 hours, at least 12 hours, at least 15 hours or at least 24 hours. In some embodiments, the minced primary tumor sample may be incubated with the enzyme mix at 25° C., 26° C., 27° C., 28° C., 29° C., 30° C., 31° C., 32° C., 33° C., 34° C., 35° C., 36° C., 37° C., 38° C., or 39° C. In some embodiments, the minced primary tumor sample may be incubated with the enzyme mix at 37° C.

In some embodiments, the minced primary tumor sample may be treated with the composition containing the enzyme in an amount or for a time sufficient yield a partial digestion of the minced primary tumor sample. In some embodiments, the minced primary tumor sample may be treated with the composition containing the enzyme for 30 minutes to 15 hours at a temperature of 25° C. to 39° C.

Collecting tumor spheroids from the enzyme mix treated sample may include centrifuging and washing the sample at least twice followed by isolating the digested tumor spheroids of the desired size. In some embodiments, the enzyme mix treated sample may be centrifuged and washed using phosphate buffered saline (PBS) at least twice. Tumor spheroids of the desired size may be collected using sieves. In some embodiments, the tumor spheroids having a diameter of 10 μm, 50 μm, 100 μm, 200 μm, 300 μm, 400 μm, 450 μm and 500 μm may be collected from the enzyme mix treated sample with the use of a sieve. In some embodiments, the tumor spheroids having a diameter of 40 m to 100 μm may be collected from the enzyme mix treated sample with the use of a sieve. In some embodiments, the tumor spheroids having a diameter of 40 μm, 50 μm, 60 μm and 70 μm may be collected from the enzyme mix treated sample with the use of a sieve.

The tumor spheroids having a desired diameter may be collected by sieving the enzyme mix treated sample through cell strainers. In some embodiments, the tumor spheroids having a diameter of 10 μm to 500 μm may be collected by sieving the enzyme mix treated sample via 500 μm and 10 μm cell strainers to yield tumor spheroids having a diameter of 10 μm to 500 In some embodiments, the tumor spheroids having a diameter of 40 μm to 100 μm may be collected by sieving the enzyme mix treated sample via 100 μm and 40 μm cell strainers to yield tumor spheroids having a diameter of 10 μm to 500 The tumor spheroids of the desired diameter may be collected and suspended in a biocompatible gel and/or extracellular matrix (ECM). Examples of biocompatible gel may include collagen, BD Matrigel™ Matrix Basement Membrane, or fibrin hydrogel (e.g., fibrin hydrogel generated from thrombin treatment of fibrinogen). Examples of ECM may include laminin, collagen IV, heparin sulfate proteoglycans, nidogen, or combinations thereof.

In some embodiments, the mixture of tumor spheroids and biocompatible gel and/or ECM may be further centrifuged.

In some embodiments, the collected tumor spheroids may be not frozen and then thawed before suspending in the biocompatible gel and/or ECM. In some embodiments, the collected tumor spheroids may be introduced into the three-dimensional cell culture device within less than 2 hours, less than 1 hour, less than 30 minutes, less than 20 minutes, less than 10 minutes, or less after collection.

In some embodiments, the collected tumor spheroids may be frozen in a freezing medium and then thawed before suspending in the biocompatible gel and/or ECM. In some embodiments, the collected tumor spheroids may be frozen for at least 6 hours 12 hours, 24 hours, 2 days, 1 week or one month. In some embodiments, the collected tumor spheroids may be frozen at −80° C. In some embodiments, the collected tumor spheroids may be frozen in liquid nitrogen. In some embodiments, the collected tumor spheroids may be frozen at −80° C. overnight, and then transferred to liquid nitrogen for storage. In some embodiments, the collected tumor spheroids may be frozen in a medium supplemented with serum. In some embodiments, the collected tumor spheroids may be frozen in a mixture containing culture medium such as DMEM or RPMI, fetal bovine serum and solvent such as Dimethyl sulfoxide (DMSO). The frozen spheroids may be thawed, for example overnight at 4° C., and then suspended in the biocompatible gel and/or ECM.

In some embodiments, the tumor spheroids may be incubated with effector cells, e.g., T cells, γδ T cells, natural killer T cells, and natural killer cells. In some embodiments, fluorophore dyes that can be used for the detection of dead cells in non-fixed conditions include, by way of example and not limitation, DNA-dependent stains such as propidium iodide, DRAQ7, and 7-AAD. In some embodiments, fluorophore dyes that can be used for the detection of live or fixed cells include, by way of example and not limitation, DNA-dependent stains, such as acridine orange, nuclear green LCS1 (ab138904), DRAQ5 (ab108410), CyTRAK Orange, NUCLEAR-ID Red DNA stain (ENZ-52406), and SiR700-DNA. Examples of non-DNA-dependent fluorophore dyes that stain for live cells are known in the art and may include, by way of example and not limitation, calcein AM, calcein violet AM, and calcein blue AM.

### Example 1

### Spheroid Formation and Comparison of Protocols—A375-RFP

Common protocol: A375 cells stably expressing red fluorescent protein (RFP) were generated by transducing A375 cells (CRL-1619™ cells obtained from ATCC) with a nucleic acid encoding RFP (A375-RFP). A375-RFP cells were grown in DMEM media (ATCC) supplemented with 10% fetal bovine serum (Avantor®). For spheroid generation, cells were trypsinized (Trypsin 0.05%, JE) and resuspended with fresh DMEM media. Cells were counted and brought to 0.5e4 cells/ml. Extra cellular matrix (ECM) reagent was added to the cell suspension at a final concentration of 2.5% v/v ECM reagent. ECM was one of Corning® Matrigel® Basement Membrane Matrix Growth Factor Reduced, Catalog No. 354230 (“Matrigel®”) (Lot: 1074009, unless otherwise indicated), GelTrex™ (Lot: 2280513) (“GelTrex™”), or Cultrex® Reduced Growth Factor Basement Membrane Matrix, Type 2 (BME 2), manufactured by Trevigen®, Catalog Number: 3533-010-02 (Lot: 1573030) (“Cultrex® BME”), which may be referred to as “BME” in the FIGS. The ECM reagent and cell suspension were gently mixed. The ECM cell suspension mixture was seeded at 200 μl per well in a 96 well ultra-low attachment (ULA) U-bottom plate (Corning® 7007) at a final cell concentration of 1e3 (1,000) cells per well. The plate was centrifuged at 125×g for 10 minutes at 4° C. The culture was monitored using an IncuCyte® live cell imaging and analysis platform for 7-10 days. On day 4 or 5 after seeding: 100 μl of media was changed with fresh DMEM media, or 100 μl of media was removed and T cells in 100 μl media were added.

New protocol (Protocol A): A375-RFP cells were grown in DMEM media (ATCC) supplemented with 10% fetal bovine serum (Avantor®). For spheroid generation, cells were trypsinized (Trypsin 0.05%, JE) and resuspended with fresh DMEM media. Cells were counted and brought to a 1e4 (10,000) cells/ml concentration. The cell suspension was seeded at 100 μl per well in a 96 well ultra-low attachment (ULA) U-bottom plate (Corning® 7007) at a final cell concentration of 1e3 (1,000) cells per well. The plate was centrifuged at 125×g for 10 minutes at 4° C. 100 μl ECM reagent was added to the cell suspension at a final concentration of 2.5% v/v. ECM was one of Matrigel® (Lot: 1074009), GelTrex™ (Lot: 2280513) or Cultrex® BME. The plate was again centrifuged at 125×g for 10 minutes at 4° C. Spheroids were allowed to form. The culture was monitored using an IncuCyte® live cell imaging and analysis platform for 7 to 10 days. On day 4 or 5 after seeding: 100 μl of media was changed with fresh DMEM media, or 100 μl of media was removed and T cells in 100 μl media were added.

Unless otherwise specified, in each Example set forth herein, images were taken, data was gathered, and calculations were made using an IncuCyte® live cell imaging and analysis platform (Model: S3-C2) and IncuCyte® S3 Spheroid Software Module. Unless otherwise specified, in each Example set forth herein, images were taken, data was gathered, and calculations were made using a 10× objective.

### Example 2

### Spheroid Formation and Comparison of Protocols—UACC257-RFP

Common protocol: UACC257 cells (obtained from ATCC) stably expressing red fluorescent protein (RFP) were generated by transducing UACC257 cells with a nucleic acid encoding RFP (UACC257-RFP). UACC257-RFP cells were grown in RPMI 1640 media (Gibco®) supplemented with 10% fetal bovine serum (Avantor®). For spheroid generation, cells were trypsinized (Trypsin 0.05%, JE) and resuspended with fresh RPMI 1640 media. Cells were counted and brought to 0.5e4 cells/ml. Extra cellular matrix (ECM) reagent was added to the cell suspension at a final concentration of 2.5% v/v ECM reagent. ECM was one of Matrigel® (Lot: 1074009), GelTrex™ (Lot: 2280513) or Cultrex® BME. The ECM reagent and cell suspension were gently mixed. The ECM cell suspension mixture was seeded at 200 μl per well in a 96 well ultra-low attachment (ULA) U-bottom plate (Corning® 7007) at a final cell concentration of 1e3 (1,000) cells per well. The plate was centrifuged at 125×g for 10 minutes at 4° C. The culture was monitored using an IncuCyte® live cell imaging and analysis platform for 7-10 days. On day 4 or 5 after seeding: 100 μl of media was changed with fresh RPMI 1640 media, or 100 μl of media was removed and T cells in 100 μl media were added.

New protocol (Protocol A): UACC257-RFP cells were grown in RPMI 1640 media (Gibco®) supplemented with 10% fetal bovine serum (Avantor®). For spheroid generation, cells were trypsinized (Trypsin 0.05%, JE) and resuspended with fresh RPMI 1640 media. Cells were counted and brought to a 1e4 (10,000) cells/ml concentration. The cell suspension was seeded at 100 μl per well in a 96 well ultra-low attachment (ULA) U-bottom plate (Corning® 7007) at a final cell concentration of 1e3 (1,000) cells per well. The plate was centrifuged at 125×g for 10 minutes at 4° C. 100 μl ECM reagent was added to the cell suspension at a final concentration of 2.5% v/v. ECM reagent. ECM was one of Matrigel® (Lot: 1074009), GelTrex™ (Lot: 2280513) or Cultrex® BME). The plate was again centrifuged at 125×g for 10 minutes at 4° C. Spheroids were allowed to form. The culture was monitored using an IncuCyte® live cell imaging and analysis platform for 7-10 days. On day 4 or 5 after seeding: 100 μl of media was changed with fresh RPMI 1640 media, or 100 μl of media was removed and T cells in 100 μl media were added.

### Example 3

### Formation of Improved Spheroids Using a New Protocol (Protocol A)—A375-RFP

Spheroids were formed using A375-RFP cells, using each of the common protocol and a new protocol (Protocol A) as set forth in Example 1. Cells were seeded in 96-well ultra-low attachment U-bottom plates (Corning® 7007), 1e3 (1,000) cells per well, with Matrigel® (Lot #1074009), Geltrex™ (Lot 2280513), or Cultrex® BME. Images were obtained every 4 hours after seeding, using an IncuCyte® instrument using a 10× objective.

FIG. 1 shows photographic images of A375-RFP cell spheroids 72 hours after seeding cells generated using either the common protocol or a new protocol (Protocol A). The first column of images shows wells containing spheroid culture produced using the common protocol, with the cells seeded with Matrigel® 2.5% v/v (Lot #1074009)(row 1), Geltrex™ 2.5% v/v (Lot #2280513) (row 2), or Cultrex® BME (row 3) 2.5% v/v. The second column of images shows wells containing spheroid culture produced using a new protocol (Protocol A), with the cells seeded with Matrigel® (row 1), Geltrex™ (row 2), or Cultrex® BME (row 3).

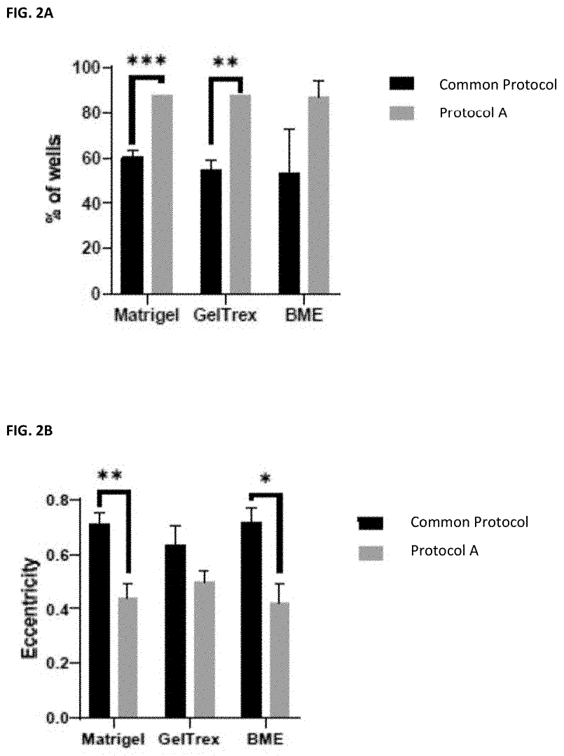

FIG. 2A and FIG. 2B show graphs comparing A375-RFP cell spheroid cultures generated using either the common protocol described herein (black bars) or a new protocol (Protocol A) described herein (grey bars), 72 hours after seeding. FIG. 2A shows the percentage of wells that could successfully be acquired and analyzed by IncuCyte® software. FIG. 2B shows the eccentricity of the spheroids, calculated using IncuCyte® software. Spheroids were analyzed using 10×objective. Asterisks represent statistical significance.

As can be seen in FIG. 1A, FIG. 1B, and FIG. 2A, and FIG. 2B, using a new protocol (Protocol A), cultures comprising a well-defined single 3D spheroid per well, with uniform morphology, better circularity, and more compact structure were generated, as compared to cultures generated using the common protocol. Due to the reduction of multiple cell clusters generated per well and central localization of these single spheroids per well, the number of wells that can be acquired and analyzed by software, such as IncuCyte® software, was increased.

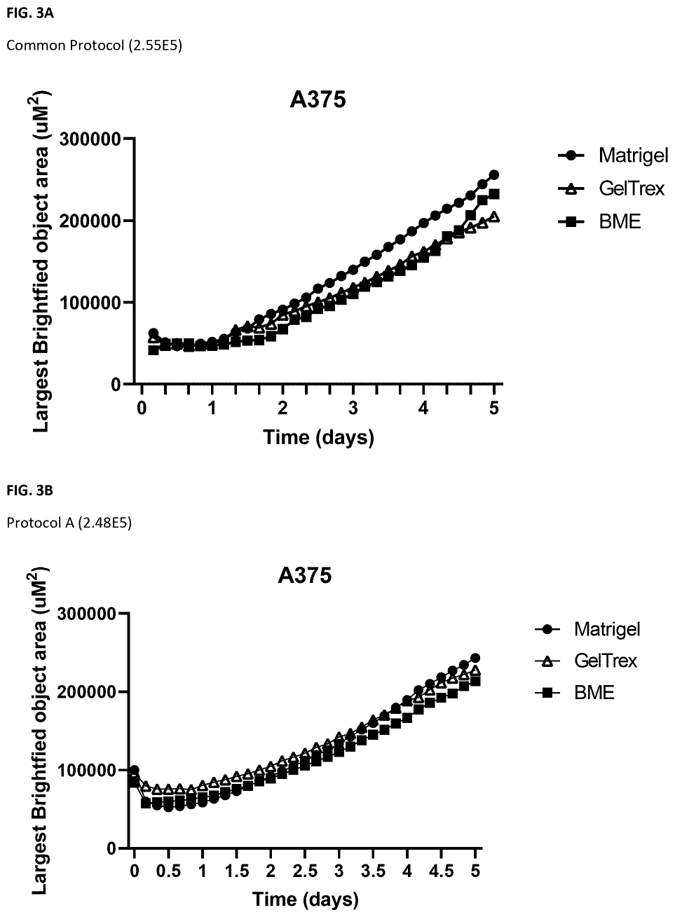

FIG. 3A and FIG. 3B show graphs comparing A375-RFP cell spheroid cultures generated using either the common protocol (FIG. 3A) or a new protocol (Protocol A) (FIG. 3B). Cells were seeded as previously set forth, 1e3 (1,000) cells per well with Matrigel®, Geltrex™, or Cultrex® BME. The size of spheroids formed by A375-RFP cells using the common protocol or a new protocol (Protocol A) was analyzed during the first 5 days of spheroid generation after seeding. Images were obtained every 4 hours after seeding on an IncuCyte® instrument using 10× objective. Spheroid size was analyzed using IncuCyte® software, according to manufacturer guidelines. Largest Brightfield Object Area in μm² (y-axis) is plotted against Time in Days (x-axis). Largest Brightfield Object Area was calculated using IncuCyte® software, using a 10× objective. Measurements were taken every 4 hours during the first 5 days after seeding. For spheroids generated using the common protocol, the Largest Brightfield Object Area measured on day 5 after seeding was 2.55E5. For spheroids generated using a new protocol (Protocol A), the Largest Brightfield Object Area measured on day 5 after seeding was 2.48E5. As can be seen, a new protocol (Protocol A) did not affect the growth rate of the spheroids as compared with the common protocol.

### Example 4

### Size Analysis of Spheroids Produced Using a New Protocol (Protocol A)—UACC257-RFP

Using a new protocol (Protocol A) as set forth in Example 2, spheroids were formed using UACC257-RFP cells (expressing preferentially expressed antigen in melanoma (PRAME)). Cells were seeded in 96-well ultra-low attachment U-bottom plates, 1e3 (1,000) cells per well. Spheroids were monitored for 4 days using an IncuCyte® system.

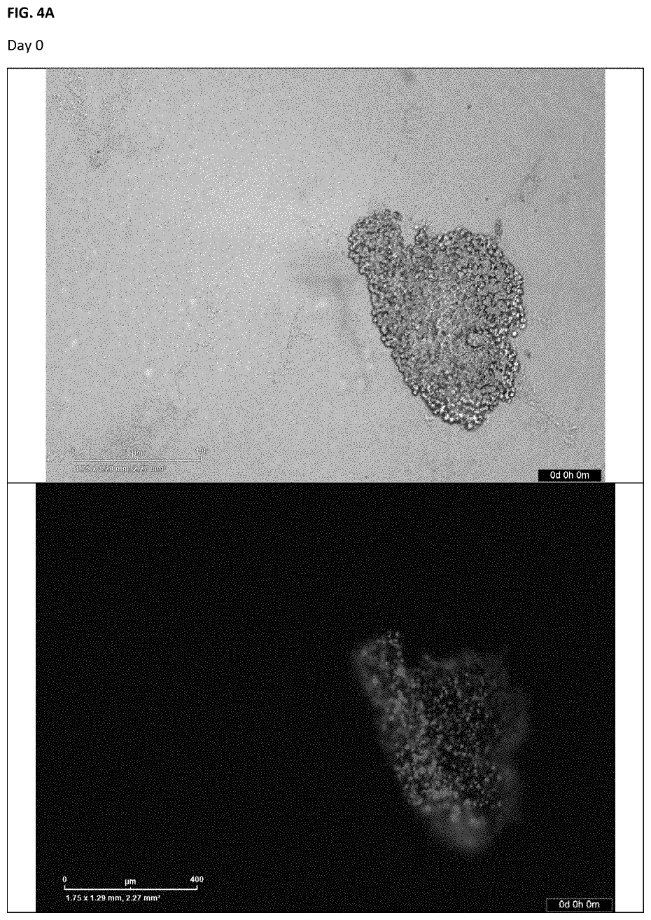

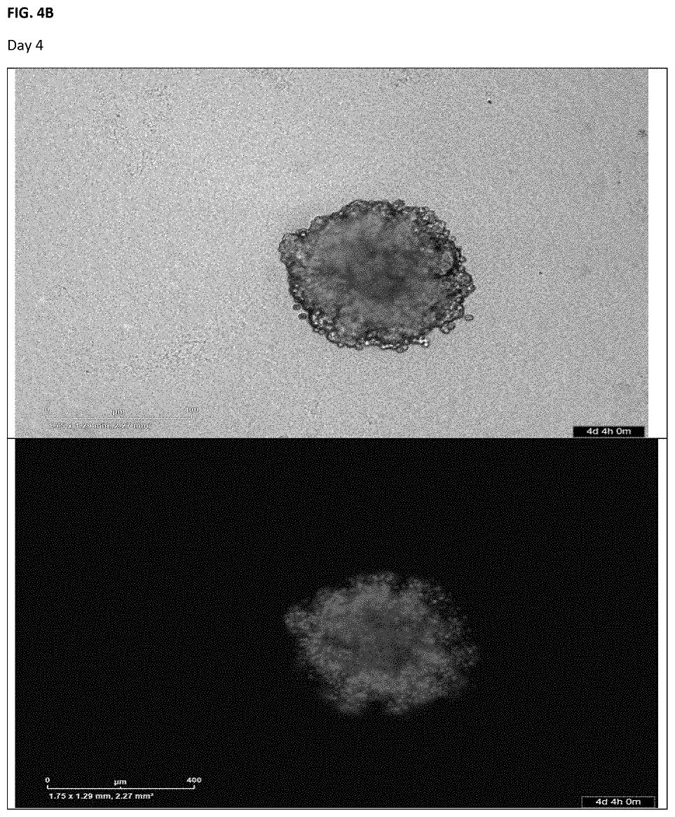

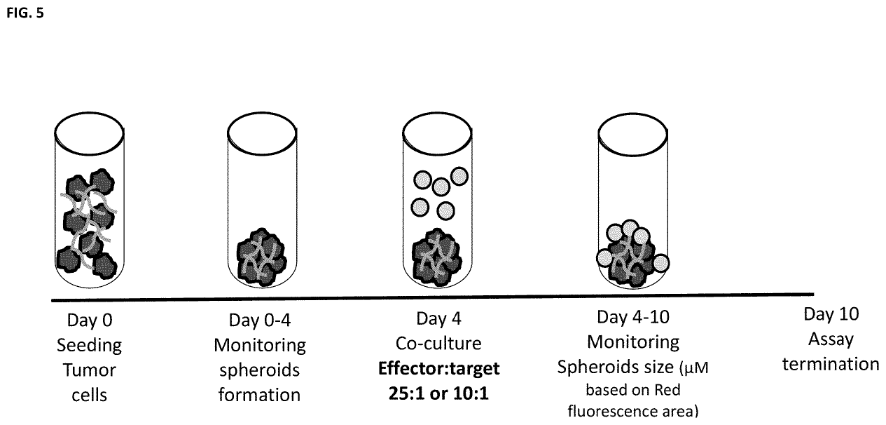

The spheroids shown in FIG. 4A and FIG. 4B were generated using Matrigel® 2.5% v/v (Lot #1074009). As shown in FIG. 4A, on Day 0 of the assay, the cells were less compact and less round than at Day 4. As shown in FIG. 4B, by Day 4 of the assay, spheroids formed and reached the size of approximately 500 μm. Images in FIG. 4A and FIG. 4B were taken using an IncuCyte® system, 10× objective, red fluorescence channel only.

### Example 5

### Spheroid Formation—T98G-RFP—Adjusted New Protocol (Protocol B)

Adjusted new protocol (Protocol B): T98G tumor cells stably expressing red fluorescent protein (RFP) were generated by transducing T98G cells (obtained from ATCC) with a nucleic acid encoding RFP (T98G-RFP). T98G-RFP cells were grown in EMEM media (ATCC) supplemented with 10% fetal bovine serum (Avantor®). For spheroid generation, cells were trypsinized (Trypsin 0.05%, JE) and resuspended with fresh RPMI 1640 media. Cells were counted and brought to a 1e4 (10,000) cells/ml concentration (for use, as a non-limiting example, when seeding at 100 μl per well) or 2e4 (20,000) cells/ml concentration (for use, as a non-limiting example, when seeding at 50 μl per well). The cell suspension was seeded at 50 μl per well or at 100 μl per well in a 96 well ultra-low attachment (ULA) U-bottom plate (Corning® 7007) at a final cell concentration of 1e3 (1,000) cells per well. The plate was centrifuged at 125×g for 10 minutes at 4° C. 50 μl (added to plates seeded with 50 μl cell suspension) or 100 μl (added to plates seeded with 50 μl cell suspension) ECM reagent was added to the cell suspension at a final concentration of 2.5% v/v Matrigel® (Lot #1074009), for a total volume of 100 μl or 200 μl. The plate was again centrifuged at 125×g for 10 minutes at 4° C. The culture was monitored using an IncuCyte® live cell imaging and analysis platform for 7-10 days. For cell cultures in 200 μl media (including ECM), on day 4 or 5 after seeding: 100 μl of media was changed with fresh EMEM media, or 100 μl of media was removed and T cells in 100 μl media were added. For cell cultures seeded in 100 μl media (including ECM), on day 4 or 5 after seeding: 100 μl of fresh EMEM media was added, or T cells in 100 μl media were added. Media was not removed from the cell cultures in 100 μl media (including ECM) before addition of T cells. Seeding cells in 50 μl, followed by centrifugation, followed by addition of 50 μl ECM reagent may be referred to as an example of an “adjusted new protocol” or “Protocol B”.

### Example 6

### Spheroid Formation—A375-RFP and UACC257-RFP—Adjusted New Protocol (Protocol B)

Spheroids were generated with each of A375-RFP cells and UACC257-RFP cells, using an adjusted new protocol (Protocol B), as described in Example 5, but using cell-appropriate media for each.

### Example 7

### Spheroid Formation—T98G-RFP—New Protocol (Protocol A)

Spheroids were generated with T98G-RFP cells using a new protocol (Protocol A), as described in Example 1, but using cell-appropriate media for T98G-RFP cells.

### Example 8

### Comparison of Spheroid Formation Using Different ECM Reagents—T98G-RFP

T98G-RFP tumor cells were prepared and spheroids allowed to form according to an adjusted new protocol (Protocol B), as in Example 5. T98G-RFP cells were seeded 1e3 (1,000) cells per well of a 96 well ultra-low attachment (ULA) U-bottom plate (Corning® 7007). Spheroids were seeded in Matrigel® (Lot 1074009 or 1077001), Geltrex™ (Lot 2280513), or Cultrex® BME). Spheroids were allowed to form after addition of 2.5% v/v ECM reagent. Images were taken and measurements were made at Day 1 and/or Day 2 after cell seeding.

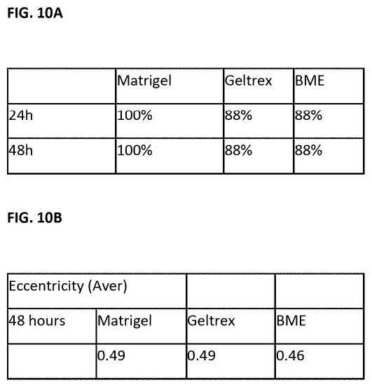

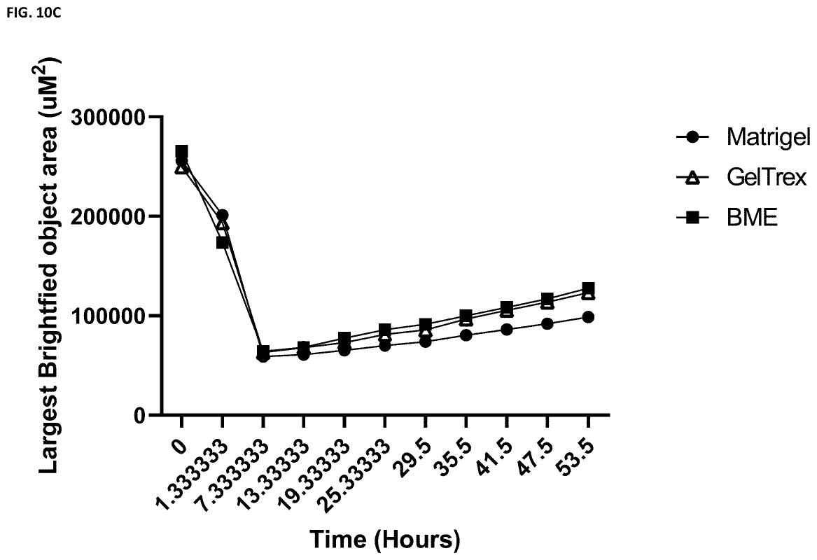

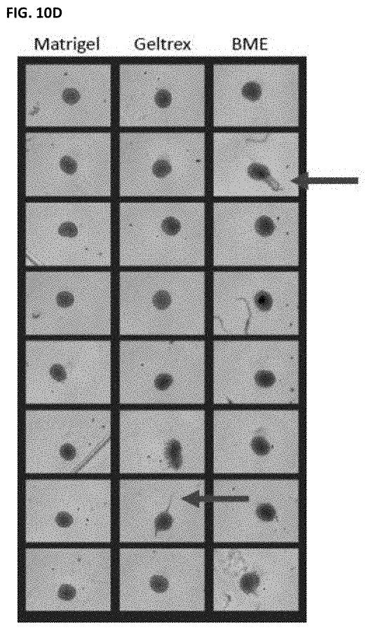

In FIG. 10A, shows the percentage of wells that could successfully be acquired and analyzed by IncuCyte® software.

FIG. 10B shows average eccentricity of spheroids calculated using IncuCyte® software.

FIG. 10C shows a graph of T98G-RFP spheroid formation. Mean Largest Brightfield Object Area in μm² (y-axis) is plotted against Time in Hours (x-axis) for 55 hours after cell seeding. As shown in FIG. 10C, spheroid formation was similar for T98G-RFP cells with Matrigel®, Geltrex™, or Cultrex® BME.

FIG. 10D shows images of T98G-RFP spheroids formed with Matrigel®, Geltrex™, or Cultrex® BME. Images were obtained using an IncuCyte® instrument using a 10× objective 48 hours after seeding the cells. The two arrows point to wells that could not be analyzed by the software; although the spheroids formed in these wells were highly compact and round, uncontrollable factors, which may be, as non-limiting examples, sterile filaments and/or proteins in the media, scratches on the plate, or a combination thereof, affected the calculations of eccentricity and the percentage of wells that could successfully be acquired and analyzed by IncuCyte® software.

### Example 9

### Comparison of Spheroid Formation Using Different ECM Products—A375-RFP, T98G-RFP, and UACC257-RFP

A375-RFP cells, T98G-RFP cells, and UACC257-RFP cells were prepared and spheroids allowed to form from each, using a new protocol (Protocol A), as in Example 1 (A375-RFP cells), Example 7 (T98G-RFP cells), or Example 2 (UACC257-RFP cells), except that each type of cell was seeded with each of Matrigel® (Lot 1074009), Geltrex™ (Lot 2280513), or Cultrex® BME.

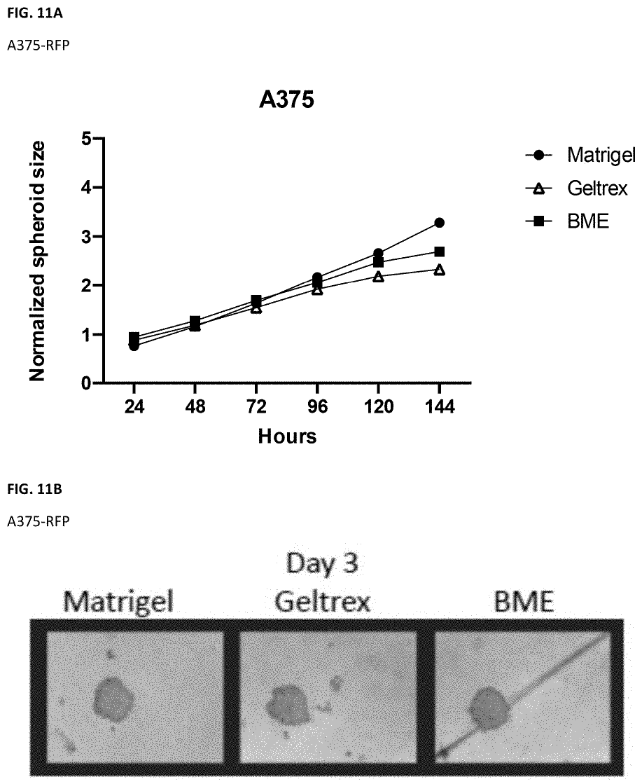

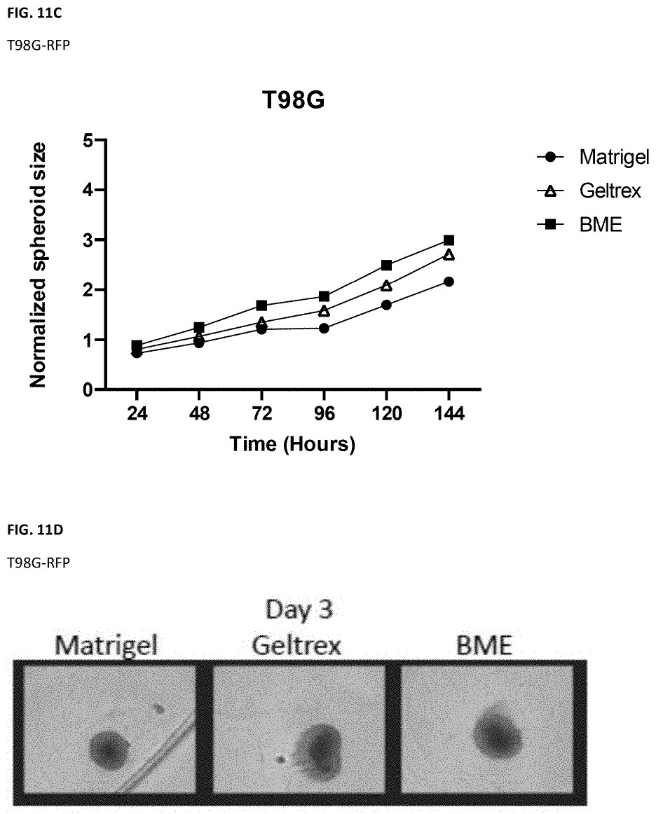

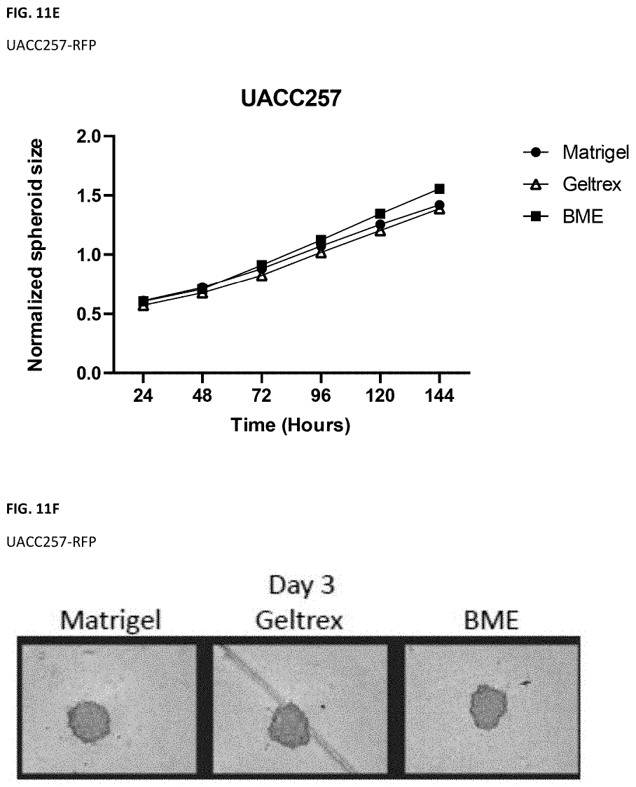

FIG. 11A shows a graph of Normalized Spheroid Size (normalized to time 0) (y-axis) plotted against Time in Hours (x-axis) for A375-RFP spheroids formed with Matrigel®, Geltrex™, or Cultrex® BME. FIG. 11B shows images of A375-RFP spheroids formed with Matrigel®, Geltrex™, or Cultrex® BME 72 hours after seeding. Images were obtained using a 10× objective. A375-RFP spheroids formed with Matrigel® showed a minor (not statistically significant) augmentation in growth rate, as compared to A375-RFP spheroids formed with Geltrex™ or Cultrex® BME.

FIG. 11C shows a graph of Normalized Spheroid Size (normalized to time 0) (y-axis) plotted against Time in Hours (x-axis) for T98G-RFP spheroids formed with Matrigel®, Geltrex™, or Cultrex® BME. FIG. 11D shows images of T98G-RFP spheroids formed with Matrigel®, Geltrex™, or Cultrex® BME 72 hours after seeding. Images were obtained using a 10× objective. T98G-RFP spheroids formed with Geltrex™ or Cultrex® BME showed a “spread” out-layer morphology, as compared to A375-RFP spheroids formed with Matrigel®.

FIG. 11E shows a graph of Normalized Spheroid Size (normalized to time 0) (y-axis) plotted against Time in Hours (x-axis) for UACC257-RFP spheroids formed with Matrigel®, Geltrex™, or Cultrex® BME. FIG. 11F shows images of UACC257-RFP spheroids formed with Matrigel®, Geltrex™, or Cultrex® BME, 72 hours after seeding. Images were obtained using a 10×objective.

These data demonstrate that spheroids generated with Matrigel® showed a slight improvement in growth rate and morphology, as compared to spheroids generated with Geltrex™ or Cultrex® BME.

### Example 10

### Comparison of Spheroid Formation Using Different Batches of Matrigel®—A375-RFP, T98G-RFP, and UACC257-RFP

A375-RFP cells, T98G-RFP cells, and UACC257-RFP cells were prepared and spheroids allowed to form from each, using a new protocol (Protocol A), as in Example 1 (A375-RFP cells), Example 7 (T98G-RFP cells), or Example 2 (UACC257-RFP cells), except that each type of cell was seeded with each of two different batches of Matrigel®: Matrigel® Lot #: 1077001 (Batch 1) (comprising 7.6 mg/ml protein concentration) or Matrigel® Lot #: 1074009 (Batch 2) (comprising 8.1 mg/ml protein concentration). Cells were seeded at 1,000 cells per well in 200 μl media.

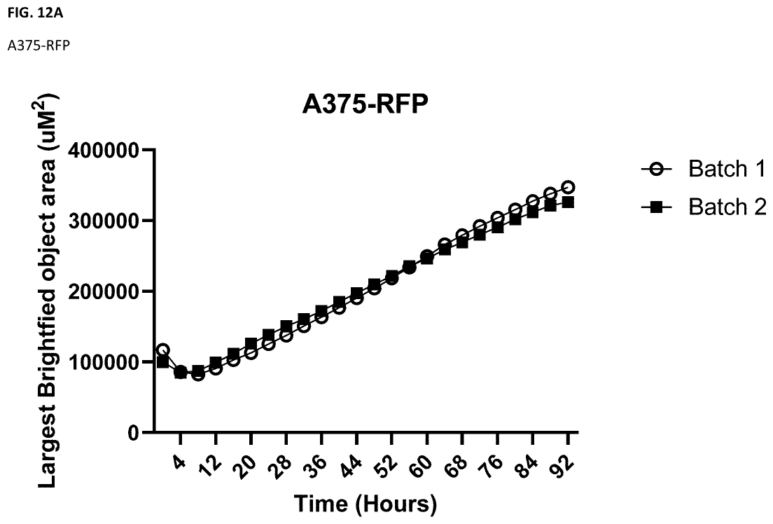

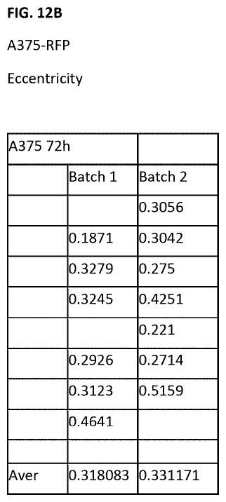

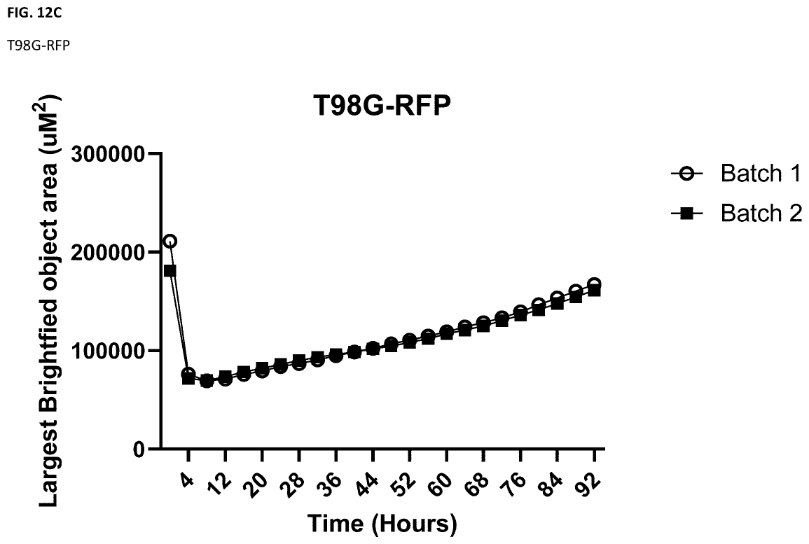

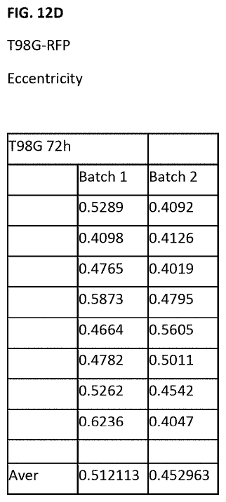

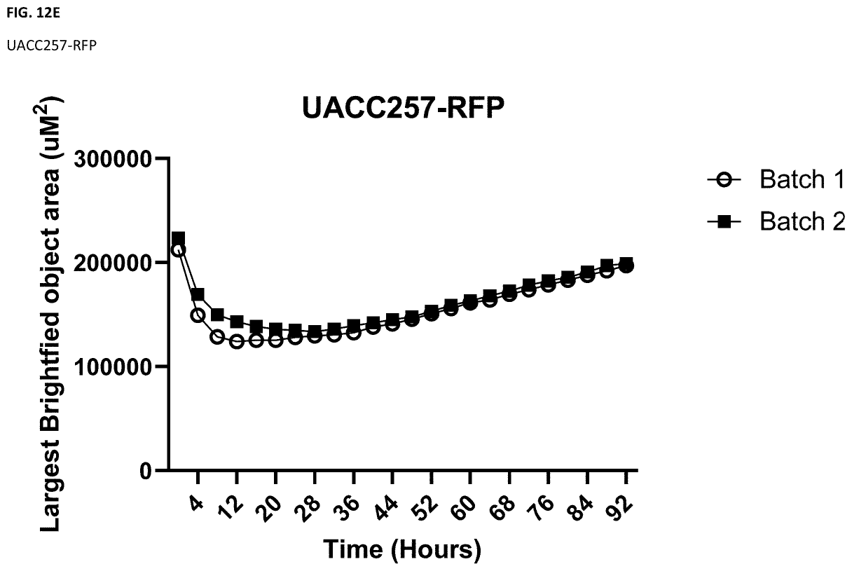

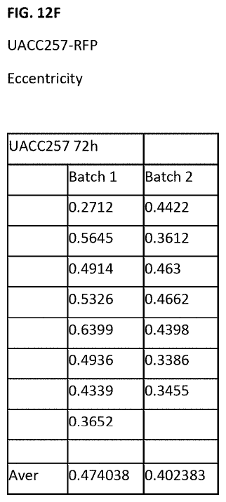

FIG. 12A shows a graph of Largest Brightfield Object Area in μm² (y-axis) plotted against Time in Hours (x-axis) for 90 hours after cell seeding for A375-RFP spheroids formed with Matrigel® Lot #: 1077001 (Batch 1) or Matrigel® Lot #: 1074009 (Batch 2). FIG. 12B shows a table of eccentricity of A375-RFP spheroids formed with Matrigel® Lot #: 1077001 (Batch 1) or Matrigel® Lot #: 1074009 (Batch 2) or. Data were obtained using a 10× objective. Eccentricity was calculated using an IncuCyte system. Data is not included for well that could not be analyzed.

FIG. 12C shows a graph of Largest Brightfield Object Area in μm² (y-axis) plotted against Time in Hours (x-axis) for 90 hours after cell seeding for T98G-RFP spheroids formed with Matrigel® Lot #: 1077001 (Batch 1) or Matrigel® Lot #: 1074009 (Batch 2). FIG. 12D shows a table of eccentricity of T98G-RFP spheroids formed with Matrigel® Lot #: 1077001 (Batch 1) or Matrigel® Lot #: 1074009 (Batch 2). Data were obtained using a 10× objective. Eccentricity was calculated using an IncuCyte system.

FIG. 12E shows a graph of Largest Brightfield Object Area in μm² (y-axis) plotted against Time in Hours (x-axis) for 90 hours after cell seeding for UACC257-RFP spheroids formed with Matrigel® Lot #: 1077001 (Batch 1) or Matrigel® Lot #: 1074009 (Batch 2). FIG. 12F shows a table of eccentricity of UACC257-RFP spheroids formed with Matrigel® Lot #: 1077001 (Batch 1) or Matrigel® Lot #: 1074009 (Batch 2). Data were obtained using a 10× objective. Eccentricity was calculated using an IncuCyte system. Data is not included for well that could not be analyzed.

No significant differences were detected between spheroids formed with different Matrigel® batches.

### Example 11

### Comparison of Spheroid Formation Using Different Volumes of Media—A375-RFP, T98G-RFP, and UACC257-RFP

To generate 200 μl cultures, A375-RFP cells, T98G-RFP cells, and UACC257-RFP cells were prepared and spheroids allowed to form from each, as in Example 1 (A375-RFP cells), as in Example 7 using anew protocol (Protocol A) (T98G-RFP cells), or as in Example 2 (UACC257-RFP cells). To generate 100 μl cultures, A375-RFP cells, T98G-RFP cells, and UACC257-RFP cells were prepared and spheroids allowed to form from each, as in Example 1 modified as in an adjusted new protocol (Protocol B) set forth in Example 5 (see Example 6) (A375-RFP cells), Example 5 using an adjusted new protocol (Protocol B) (T98G-RFP cells), or Example 2 modified as in an adjusted new protocol (Protocol B) set forth in Example 5 (see Example 6) (UACC257-RFP cells). Cells were seeded at 1,000 cells per well. In each culture, spheroids were formed with Matrigel® 2.5% v/v. Spheroids were imaged on Day 3 after seeding, using a 10× objective.

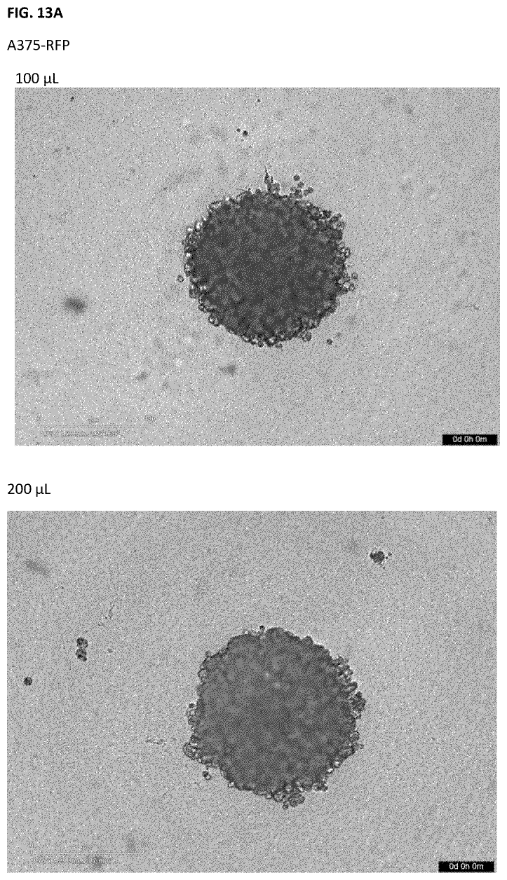

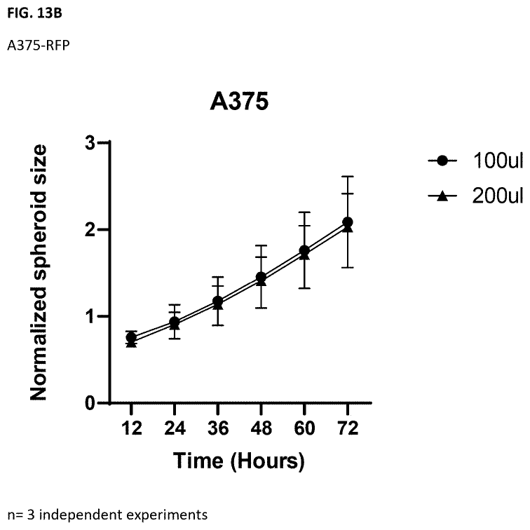

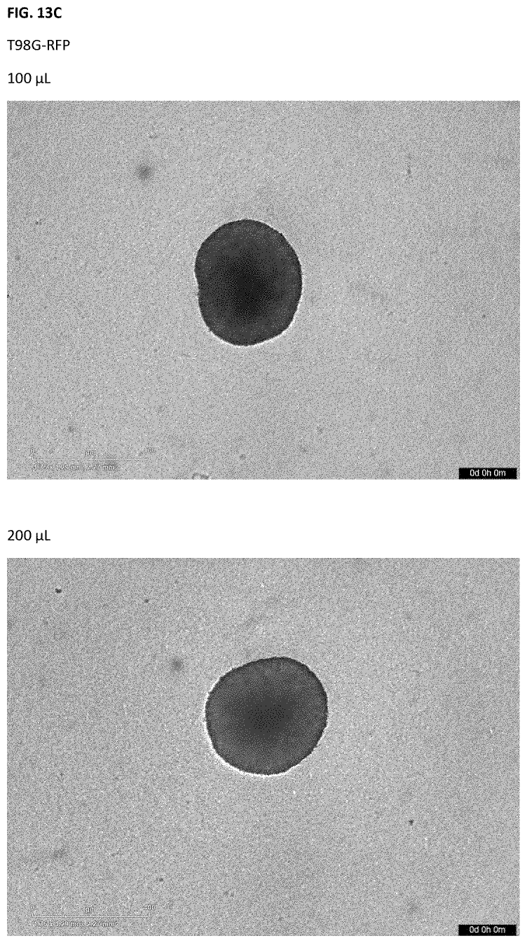

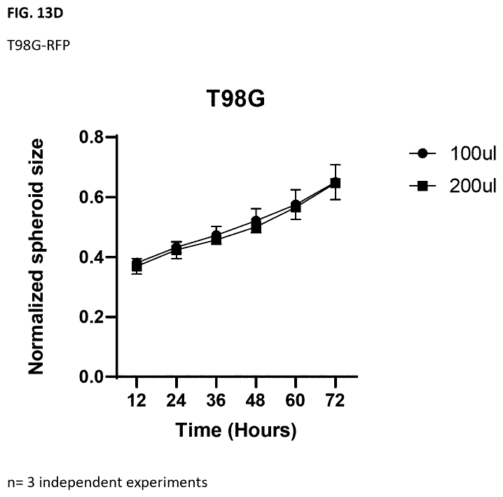

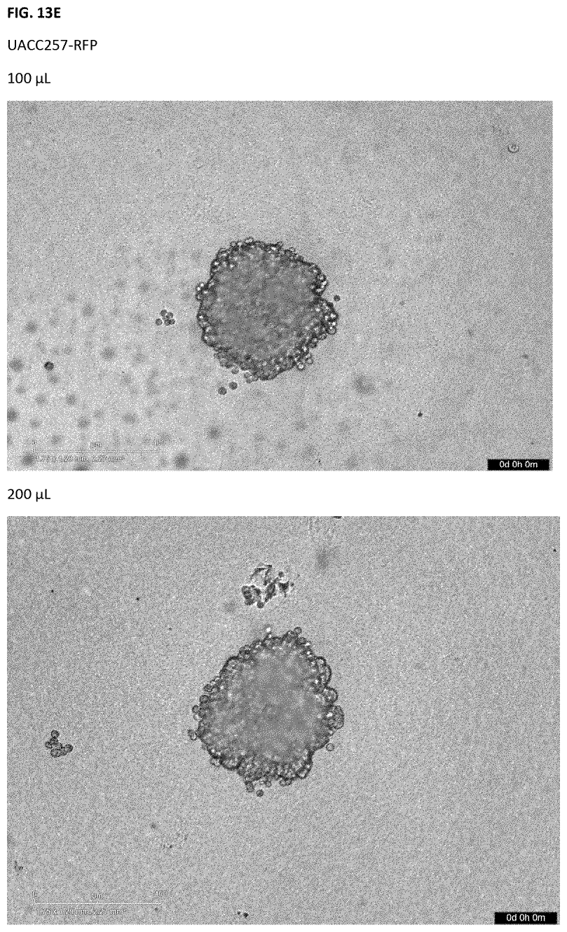

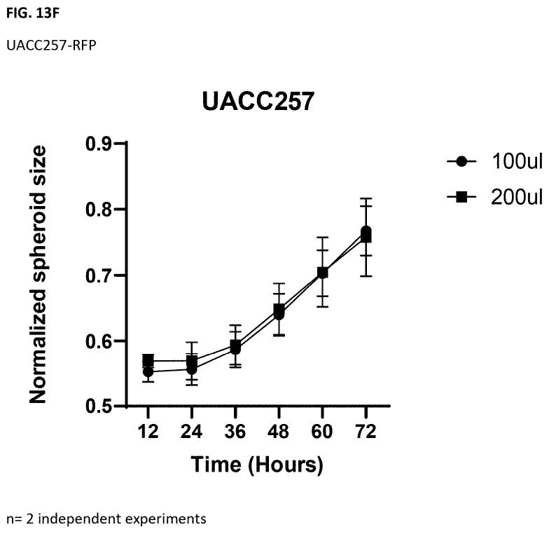

FIG. 13A shows images of A375-RFP spheroids formed with 100 μl or 200 μl media. FIG. 13B shows a graph Normalized Spheroid Size (normalized to time 0) (y-axis) plotted against Time in Hours (x-axis) for A375-RFP spheroids formed with 100 μl or 200 μl media. These data are the average of three independent experiments.

FIG. 13C shows images of T98G-RFP spheroids formed with 100 μl or 200 μl media. FIG. 13D shows a graph Normalized Spheroid Size (normalized to time 0) (y-axis) plotted against Time in Hours (x-axis) for T98G-RFP spheroids formed with 100 μl or 200 μl media. These data are the average of three independent experiments.

FIG. 13E shows images of UACC257-RFP spheroids formed with 100 μl or 200 μl media. FIG. 13F shows a graph Normalized Spheroid Size (normalized to time 0) (y-axis) plotted against Time in Hours (x-axis) for UACC257-RFP spheroids formed with 100 μl or 200 μl media. These data are the average of two independent experiments.

These data demonstrate similar morphology and growth rate for spheroids formed in 100 μl media, as compared to spheroids formed in 200 μl media.

### Example 12

### Comparison of Spheroid Formation Using Different Concentrations of ECM—A375-RFP, T98G-RFP, and UACC257-RFP

A375-RFP cells, T98G-RFP cells, and UACC257-RFP cells were prepared and spheroids allowed to form from each, using a new protocol (Protocol A), as in Example 1 (A375-RFP cells), Example 7 (T98G-RFP cells), or Example 2 (UACC257-RFP cells), except that ECM was added to each well to a concentration of either 1% v/v or 2.5% v/v Matrigel® (Lot #1074009). Cells were seeded at 1,000 cells per well. Spheroids were imaged on Day 4 after seeding using an IncuCyte® instrument using a 10×objective, brightfield.

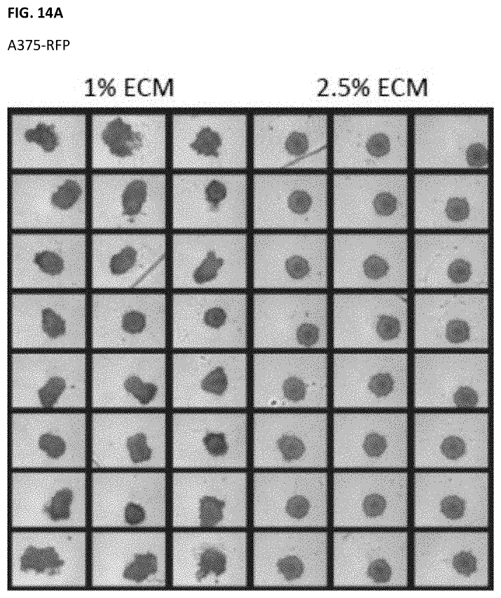

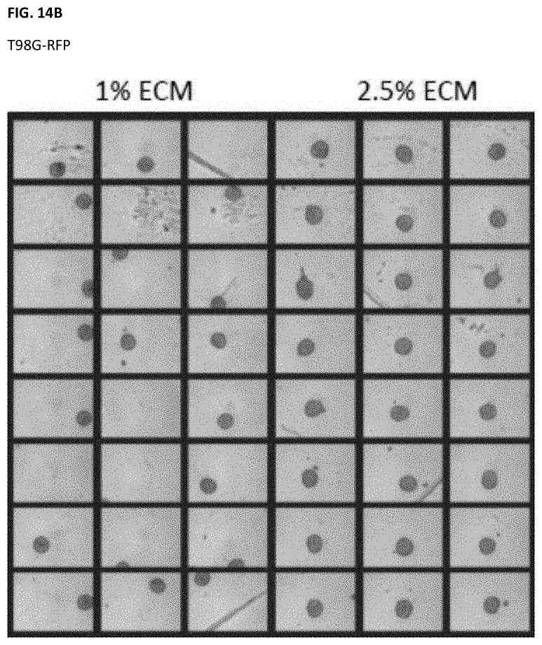

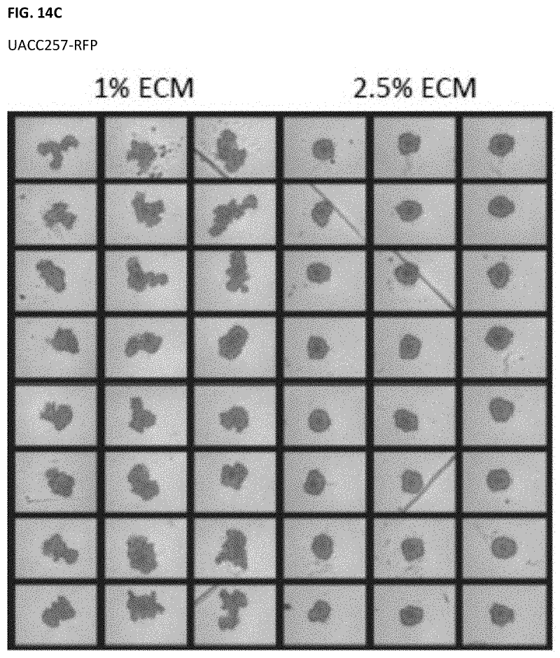

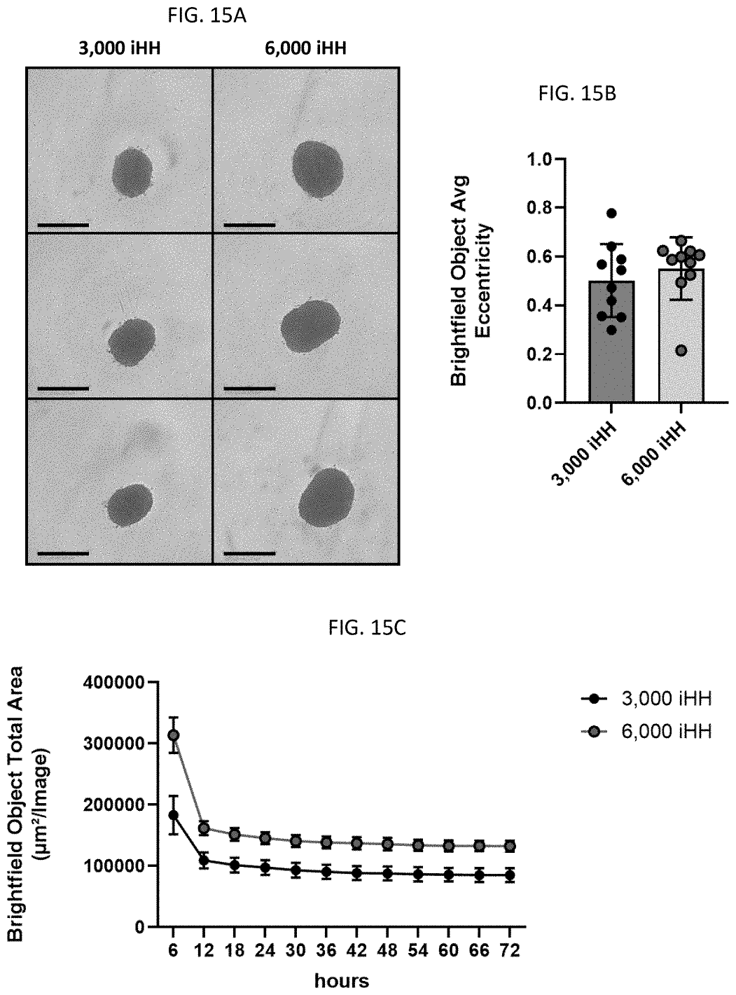

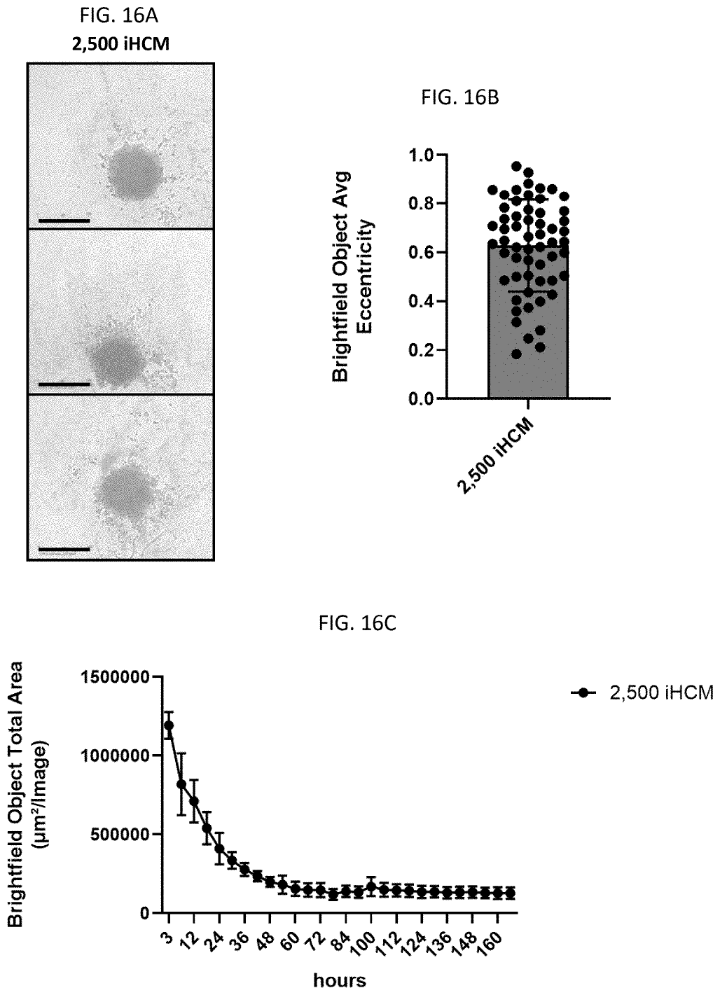

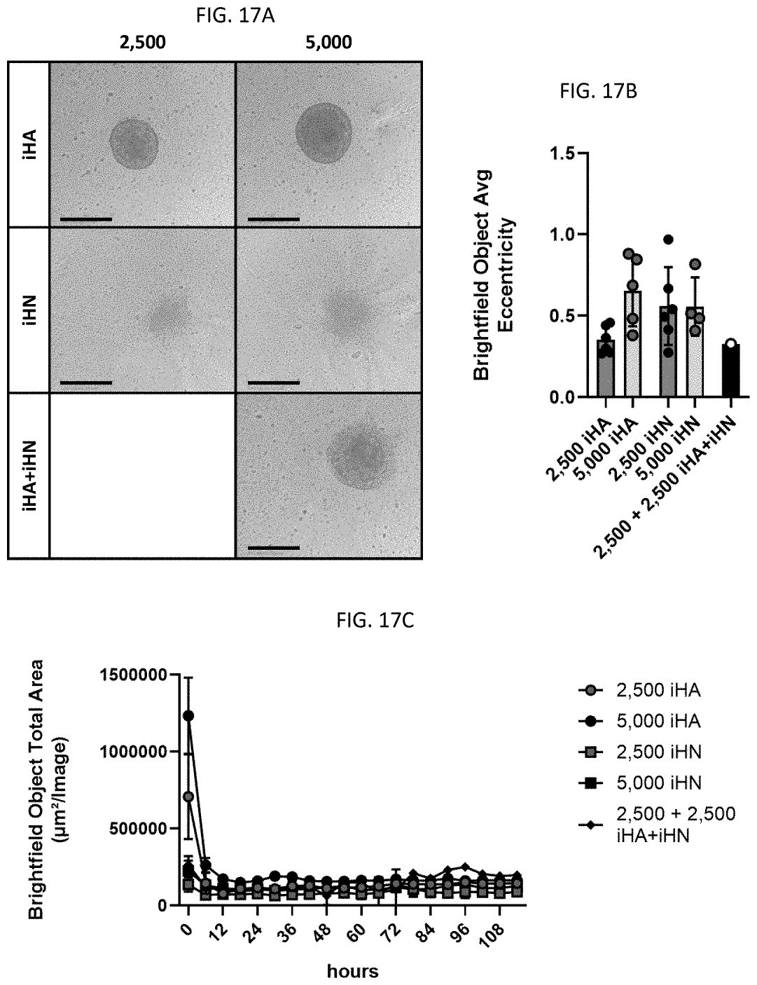

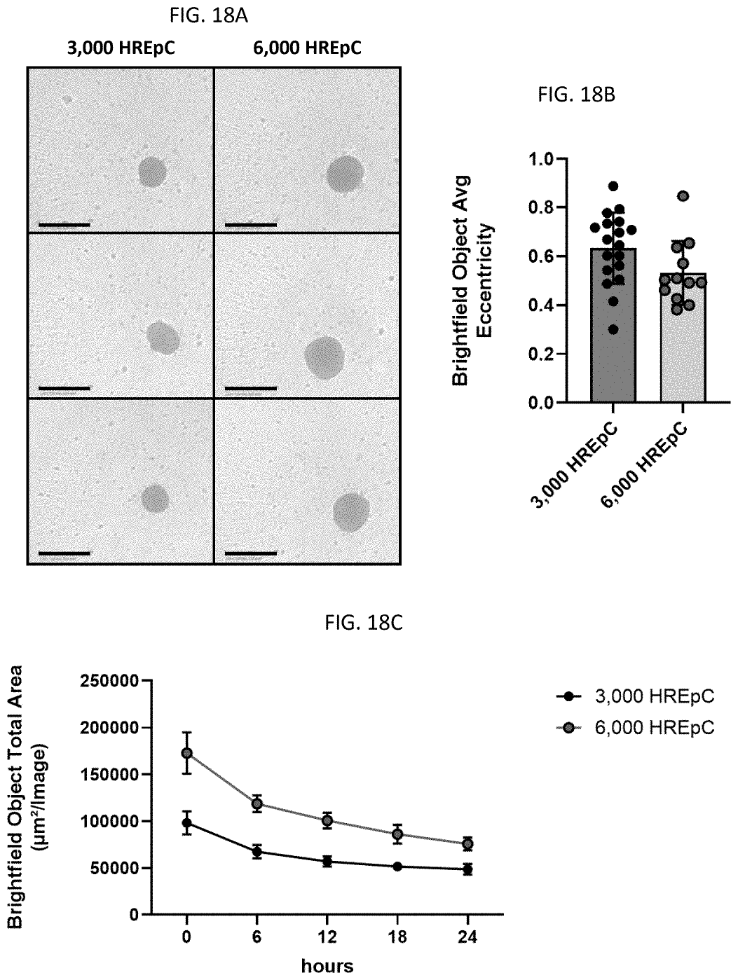

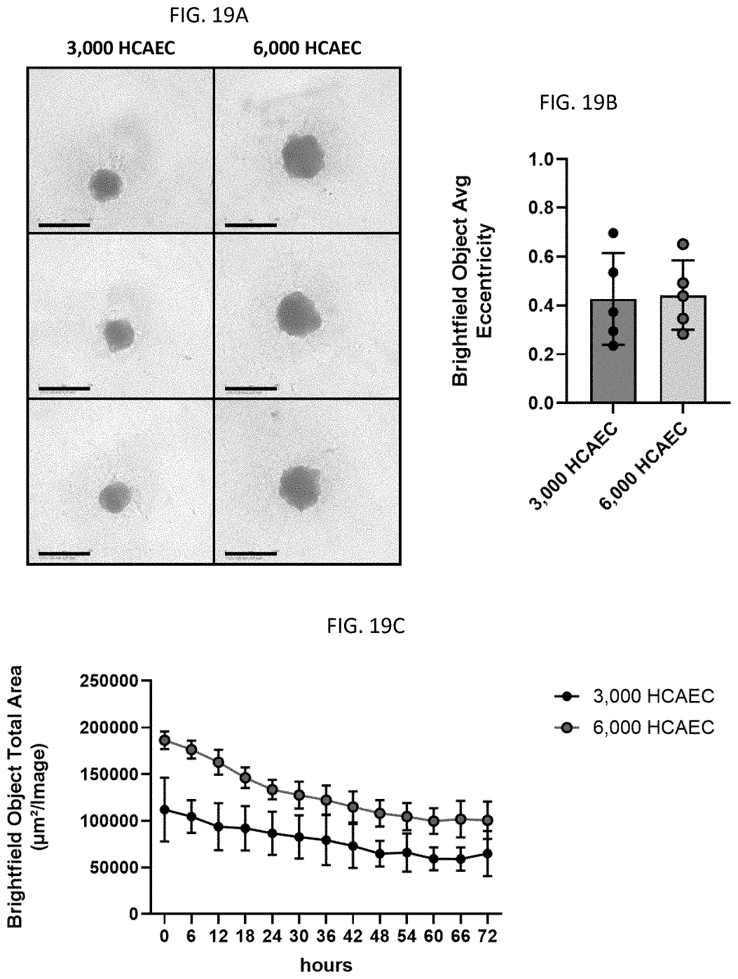

FIG. 14A shows images of A375-RFP spheroids formed with 1% or 2.5% Matrigel®.

FIG. 14B shows images of T98G-RFP spheroids formed with 1% or 2.5% Matrigel®.

FIG. 14C shows images of UACC257-RFP spheroids formed with 1% or 2.5% Matrigel®.

These data demonstrate that reduction of Matrigel® concentration from 2.5% to 1% impaired spheroid morphology.

### Example 13

### CD3⁺ T Cell Killing of Spheroids Generated Using an Adjusted New Protocol (Protocol B)—UACC257-RFP

T cell killing assays were performed on spheroids formed using PRAME-expressing UACC257-RFP tumor cells. Cells were prepared using an adjusted new protocol (Protocol B) (as in Example 1 modified as in an adjusted new protocol (Protocol B) set forth in Example 5 (see Example 6)) and spheroids allowed to form. Matrigel® (Lot #1074009) 2.5% v/v was used. T cells were prepared using peripheral blood mononuclear cells (PBMC's) from one healthy donor.

T cells were transduced with one of R11KEA TCR WT (TCR), CD8αβ.TCR, or CD8α.TCR, in which the TCR specifically binds PRAME/MHC complex. CD8αβ.TCR expresses CD8αβ heterodimer and R11KEA TCR WT. CD8α.TCR expresses CD8αCD8βstalk with CD8α transmembrane and intracellular domain (CD8α homodimer) and R11KEA TCR WT.

T cells were separated into CD4⁺ and CD8⁺ populations using CD4⁺ selection beads (Miltenyi®), and a comparison was conducted on each population, including total CD3⁺ population (PBMC's product). The Effector to Target ratios examined were 25:1 and 10:1 (adjusted to the Tee population, based on Flow cytometry staining). The amount of T cells was calculated relative to the initial seeding density of the tumor cells (amount of tumor cells at day 0) (1,000 cells per well). Spheroid killing assays were performed on Day 4 of spheroid growth. Spheroid size (in μm², based on red fluorescent area) was monitored on days 0-8 or 0-11. Killing analysis was performed based on the size of the largest red fluorescent area. FIG. 5 shows a schematic of a non-limiting exemplary killing assay that may be used in embodiments.

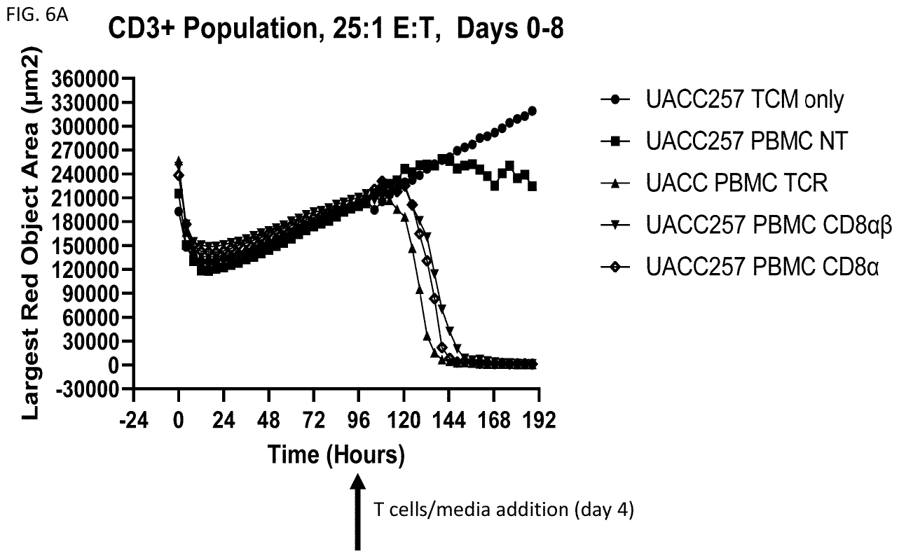

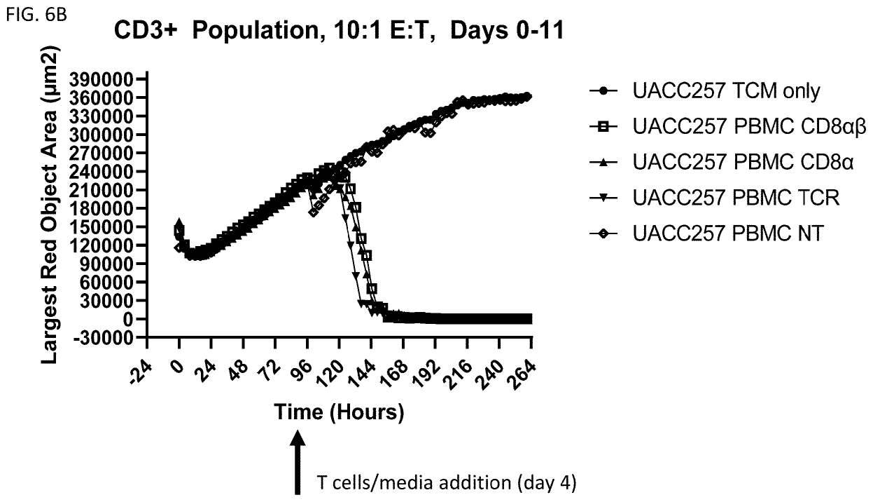

T cell media, non-transduced CD3⁺ PBMC cells, or CD3⁺ T cells transduced with one of R11KEA TCR WT (TCR), CD8αβ.TCR, or CD8α.TCR were added to UACC257-RFP spheroids 4 days after culture initiation. Cells were added at a concentration of 10,000 or 25,000 cells per well. Spheroid size was monitored for at least an additional 4 days. FIG. 6A shows an Effector to Target ratio of 25:1 (25,000 T cells: 1,000 tumor cells (based on day 0 seeding amount)), while FIG. 6B shows an Effector to Target ratio of 10:1 (10,000 T cells: 1,000 tumor cells (based on day 0 seeding amount). Based on recent experiments evaluating the number of tumor cells per spheroid on day 4, the actual Effector to Target ratios may be lower, such as 3:1 for a nominal 10:1 ratio, as the tumor cells proliferate during the first 4 days after seeding before addition of T cells. FIG. 6A and FIG. 6B show the Largest Red Object Area in μm² (y-axis) plotted against Time in Days (x-axis) for 8 (FIG. 6A) or 11 (FIG. 6B) days after cell seeding. Largest Red Object Area was measured using an IncuCyte® system, 10×objective, red fluorescence channel only. FIG. 6A and FIG. 6B show that similar killing capabilities were demonstrated by CD3⁺ T cells transduced with the three different TCR constructs, R11KEA TCR WT (line indicated by arrow labelled “UACC257 PBMC TCR”), CDαβ.TCR (line indicated by arrow labelled “UACC257 PBMC CD8αβ.”), or CD8α.TCR (line indicated by arrow labelled “UACC257 PBMC CD8α.”); but T cell media (line indicated by arrow labelled “UACC257 TCM only”) and non-transduced PBMC cells (line indicated by arrow labelled “UACC257 PBMC NT”) did not have similar killing capabilities as compared to the cells transduced with TCR. Transduced CD3⁺ cells demonstrated high killing capability at both Effector to Target ratios 25:1 (FIG. 6A) and 10:1 (FIG. 6B). TCR⁺ cells showed the most potent effect on spheroid size. The examined transduced CD3⁺ conditions showed comparable killing capabilities. Non-transduced cells and media control did not demonstrate any killing.

### Example 14

### CD8⁺ T Cell Killing of Spheroids Generated Using an Adjusted New Protocol (Protocol B) UACC257-RFP

T cell killing assays were performed on spheroids formed using PRAME-expressing UACC257-RFP tumor cells. Cells were prepared and spheroids allowed to form, using an adjusted new protocol (Protocol B) (as in Example 1 modified as in an adjusted new protocol (Protocol B) set forth in Example 5 (see Example 6)). Matrigel® (Lot #1074009) 2.5% v/v was used. T cells were prepared using peripheral blood mononuclear cells (PBMC's) from one healthy donor.

T cells were transduced with one of R11KEA TCR WT (TCR), CD8αβ.TCR, or CD8α.TCR T cells.

T cells were separated into CD4⁺ and CD8⁺ populations using CD4⁺ selection beads (Miltenyi®), and a comparison was conducted on each population, including total CD3⁺ population (PBMC's product). The Effector to Target ratios examined were 25:1 and 10:1 (adjusted to the Tee population, based on Flow cytometry staining). The amount of T cells was calculated relative to the initial seeding density of the tumor cells (amount of tumor cells at day 0) (1,000 cells per well). Spheroid killing assays were performed on Day 4 of spheroid growth. Spheroid size (in μm², based on red fluorescence area) was monitored on days 0-9 or 0-10. Killing analysis was performed based on the size of the largest red fluorescent area. FIG. 5 shows a schematic of a non-limiting exemplary killing assay that may be used in embodiments.

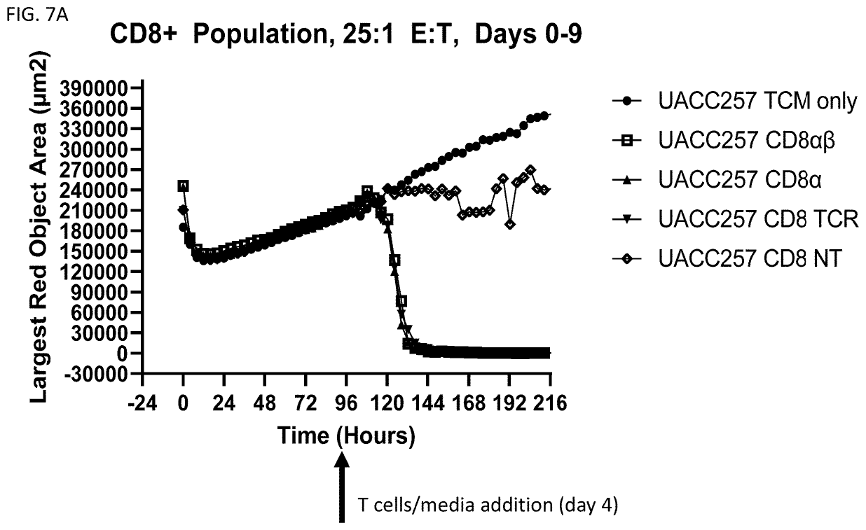

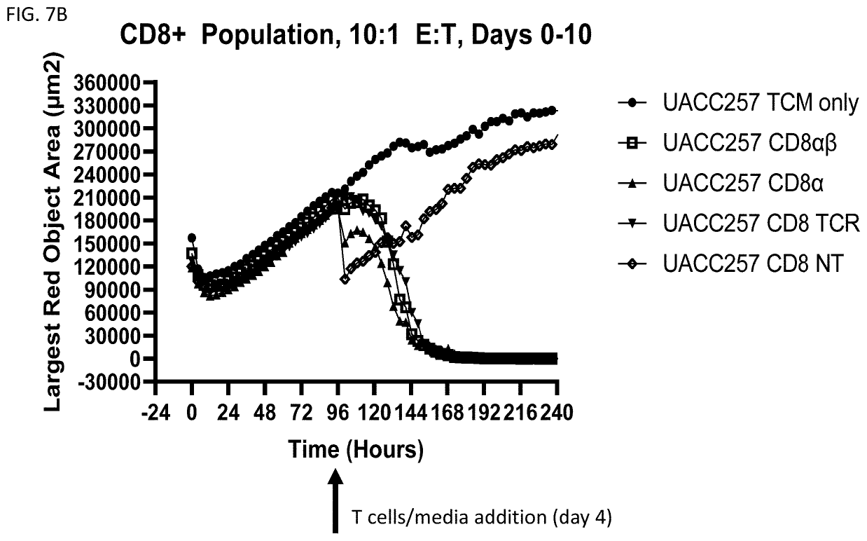

T cell media, non-transduced CD8⁺ PBMC cells, or CD8⁺ T cells transduced with one of R11KEA TCR WT, CD8αβ.TCR, or CD8α.TCR were added to UACC257-RFP spheroids 4 days after culture initiation. Cells were added at a concentration of 1,000 cells per well. Spheroid size was monitored for at least an additional 5 days. FIG. 7A shows an Effector to Target ratio of 25:1, while FIG. 7B shows an Effector to Target ratio of 10:1. Based on recent experiments, the actual Effector to Target ratios may be lower, such as 3:1 for a nominal 10:1 ratio, as the tumor cells proliferate during the first 4 days after seeding before addition of T cells. FIG. 7A and FIG. 7B show the Largest Red Object Area in μm² (y-axis) plotted against Time in Days (x-axis) for 9 (FIG. 7A) or 10 (FIG. 7B) days after cell seeding. Largest Red Object Area was measured using an IncuCyte® system, 10×objective, red fluorescence channel only. FIG. 7A and FIG. 7B show that similar killing capabilities were demonstrated by CD8⁺ T cells transduced with the three different TCR constructs, R11KEA TCR WT (line indicated by arrow labelled “UACC257 CD8 TCR”), CDαβ.TCR (line indicated by arrow labelled “UACC257 CD8αβ.”), or CD8α.TCR (line indicated by arrow labelled “UACC257 CD8α.”); but T cell media (line indicated by arrow labelled “UACC257 TCM only”) and non-transduced PBMC cells (line indicated by arrow labelled “UACC257 CD8 NT”) did not have similar killing capabilities as compared to the cells transduced with TCR. Transduced CD8⁺ cells demonstrated high killing capability at both Effector to Target ratios 25:1 (FIG. 7A) and 10:1 (FIG. 7B). The examined transduced CD8⁺ conditions showed comparable killing capabilities. Non-transduced cells and media control did not demonstrate any killing.

### Example 15

### T Cell Killing of Spheroids Generated Using an Adjusted New Protocol (Protocol B)—UACC257-RFP

T cell killing assays were performed on spheroids formed using PRAME-expressing UACC257-RFP tumor cells. UACC257-RFP cells were prepared and spheroids allowed to form, using an adjusted new protocol (Protocol B) (as in Example 1 modified as in an adjusted new protocol (Protocol B) set forth in Example 5 (see Example 6)). Cells were seeded at 1,000 cells per well. Spheroids were formed with Matrigel® (Lot #1074009) 2.5% v/v. T cells were prepared using peripheral blood mononuclear cells (PBMC's) from three healthy donors.

T cell media, non-transduced CD4+ T cells, or CD4⁺ T cells transduced with one of R11KEA WT TCR (TCR), CD8αβ.TCR, or CD8α.TCR at an Effector: Target ratio of 4:1 (effectors normalized to % CD3⁺ Tet⁺) were added to UACC257-RFP spheroids 4 days after culture initiation. Cells were co-cultured for 6 days.

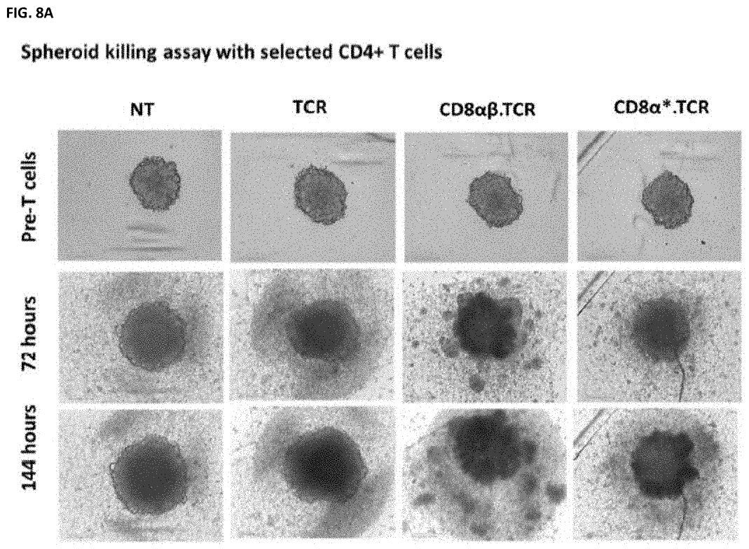

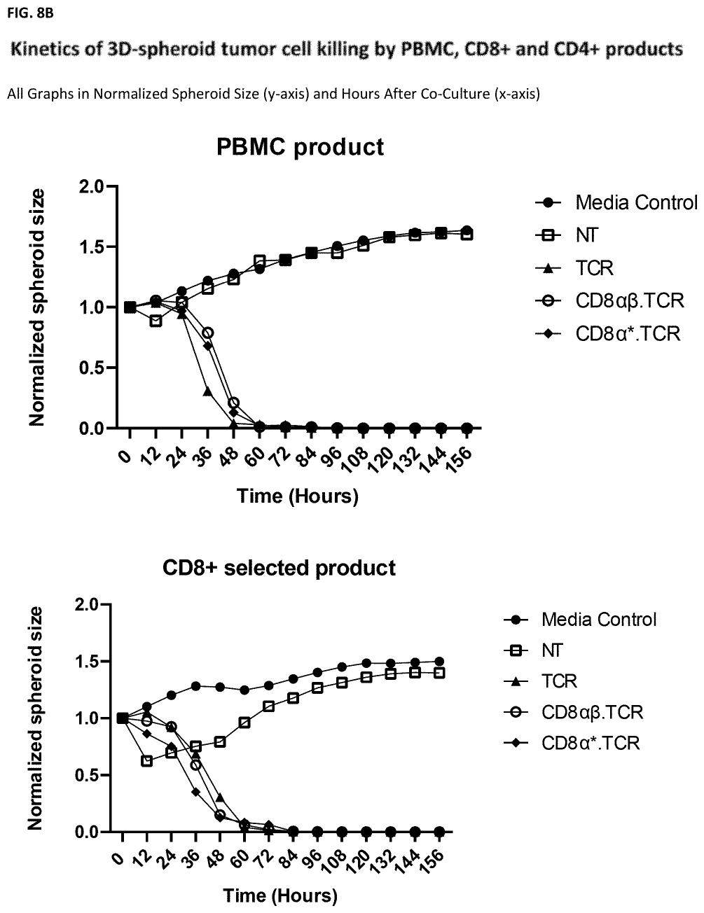

FIG. 8A shows representative bright field images of UACC257-RFP spheroids before T cells were added, and 72 and 144 hours after CD4+ T cells were added. Images were obtained using a 10× objective.

FIG. 8B shows graphs of spheroid size analysis over 156 hours post addition of PBMC-derived products transduced with the indicated different receptors, CD8⁺ selected T cells transduced with the indicated different receptors, or CD4⁺ selected T cells transduced with the indicated different receptors. Data shown is normalized to last acquired time point prior to T cell addition. Data shown is for one of three donor products.

The data show that CD4⁺ T cells expressing R11KEA TCR and CD8αβ heterodimer (CD8αβ.TCR) exhibit greater killing activity against PRAME+ tumor cells, e.g., UACC257, than CD4⁺ T cells expressing CD8α homodimer (CD8α.TCR). Similar to the negative controls, e.g., the non-transduced CD4⁺ cells (NT) and the media control, CD4⁺ T cells expressing R11KEA TCR alone (TCR, no CD8 receptor) did not exhibit killing activity, suggesting that the expression of CD8αβ heterodimer or CD8α homodimer in CD4⁺ T cells may be required to induce PRAME-specific cytotoxic activity of CD4⁺ T cells expressing R11KEA TCR. As a control, CD8⁺ T cells or PBMC expressing CD8αβ.TCR, CD8α.TCR, or R11KEA TCR alone (TCR) exhibit comparable killing activity against UACC257.

### Example 16

### T Cell Killing of Spheroids Generated Using an Adjusted New Protocol (Protocol B)—UACC257-RFP

T cell killing assays were performed on spheroids formed using PRAME-expressing UACC257-RFP tumor cells. UACC257-RFP tumor cells were prepared and spheroids allowed to form, using an adjusted new protocol (Protocol B) (as in Example 1 modified as in an adjusted new protocol (Protocol B) set forth in Example 5 (see Example 6)), using Matrigel® (Lot #1077001) 2.5% v/v. T cells were prepared using Peripheral blood mononuclear cells (PBMC's) from one healthy donor.

CD4⁺ T cells and CD8⁺ T cells transduced with CD8αβ.TCR at an Effector:Target ratio of 25:1 to UACC257-RFP (seeded at 1,000 cells per well) spheroids 4 days after culture initiation.

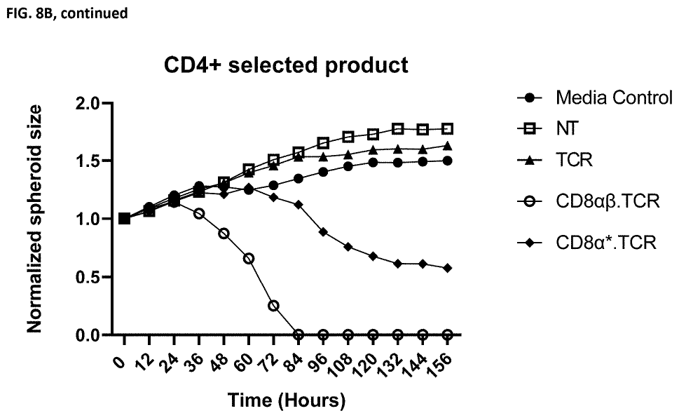

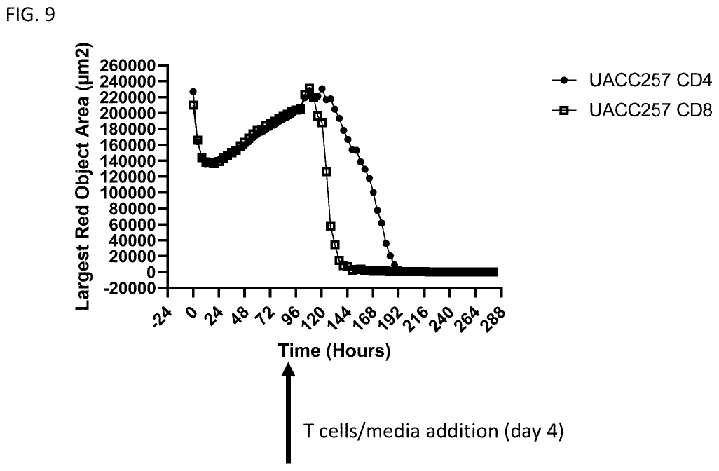

FIG. 9 shows a graph of the Largest Red Object Area in μm² (y-axis) against Time (in Days) (x-axis) for 10 days after cell seeding of UACC257-RFP spheroids challenged with either CD4⁺ T cells transduced with CD8αβ.TCR (“UACC257 CD4”) or CD8⁺ T cells transduced with CD8αβ.TCR (“UACC257 CD8”). As shown in FIG. 9, CD8αβ.TCR CD8⁺ cells had faster and stronger killing kinetics compared to CD8αβ.TCR CD4⁺ cells.

### Example 17

### T Cell Killing of Spheroids Generated Using an Adjusted New Protocol (Protocol B)—UACC257-RFP Cells—Summary

Overall, the data presented in the Figures discussed above show that: TCR transduced CD3⁺ cells have demonstrated a pronounced killing capability, and no significant differences were detected between the transduced constructs. No difference in spheroid killing by CD8⁺ T cells was observed comparing the three TCR constructs. CD4⁺ cells acquired killing ability when transduced with TCR⁺ CD8⁺ constructs. CD4⁺ T cells transduced with CD8αβ.TCR construct have greater potential for killing capability than those transduced with CD8α.TCR. CD4⁺ cells demonstrated a reduced killing effect compared with CD8⁺ cells transduced with CD8αβ.TCR.

### Example 18

### Spheroid Formation—Primary Human Cells—Protocol B

Spheroid Formation—Human iPSC-Derived Hepatocytes (iHH)—Protocol B

Protocol B: Hepatocytes 2.0 (iHH) were cultured according to the manufacture's application protocol “Modeling 3D Liver Tissue: 3D Hepatocyte Spheroids in Low Attachment Plates” (FUJIFILM Cellular Dynamics, Inc). For spheroid generation, cells were handled according to the application protocol until step 12 of “Harvesting Hepatocytes”. The cell suspension was seeded at 35 μl per well in a 96 well ultra-low attachment (ULA) U-bottom plate (Corning® 4515 or Thermo Scientific™ 174925). The plate was centrifuged at 200×g for 2 minutes at 4° C. ECM solution was prepared on ice and all tips and reservoirs were precooled and kept cold during the whole procedure. 35 μl of ECM solution was added to the wells at a final concentration of 2.5% v/v Geltrex™, for a total volume of 70 μl. The plate was again centrifuged at 200×g for 2 minutes at 4° C. The culture was monitored using an IncuCyte® live cell imaging and analysis platform for a maximum of 7 days. 80% of the culture medium was changed with fresh medium every other day. Seeding cells in 35 μl, followed by centrifugation, followed by addition of 35 μl ECM solution may be referred to as an example of an “Protocol B”. (FIGS. 26 and 27).

Spheroid Formation—Human iPSC-Derived cardiomyocytes (iHCM)—Protocol B

Protocol B: iCell® Cardiomyocytes² (iHCM) were thawed according to the manufacture's protocol “User's Guide” (FUJIFILM Cellular Dynamics, Inc). For spheroid generation, cells were counted and the cell suspension was seeded at 2,500 cells in 40 μl per well in a 96 well ultra-low attachment (ULA) U-bottom plate (Corning® 4515 or Thermo Scientific™ 174925). The plate was centrifuged at 300×g for 5 minutes at 4° C. ECM solution was prepared on ice and all tips and reservoirs were precooled and kept cold during the whole procedure. 40 μl of ECM solution was added to the cell suspension at a final concentration of 2.5% v/v Geltrex™, for a total volume of 80 μl. The plate was again centrifuged at 300×g for 5 minutes at 4° C. The culture was monitored using an IncuCyte® live cell imaging and analysis platform for a maximum of 7 days. 80% of the culture medium was changed with fresh medium every other day. Seeding cells in 40 μl, followed by centrifugation, followed by addition of 40 μl ECM solution may be referred to as an example of an “Protocol B”.

Spheroid Formation—Human iPSC-Derived astrocytes (iHA) and human iPSC-derived GABA-neurons (iHN)—Protocol B

Protocol B: Astrocytes (iHA) and iCell® GABANeurons (iHN) were thawed according to the respective manufacture's protocols “Quick Guide” and “User's Guide” (FUJIFILM Cellular Dynamics, Inc). For spheroid generation, cells were counted and the cell suspension was seeded at 2,500 or 5,000 cells in 50 μl or 2,500 in 25 μl+25 μl for the co-culture per well in a 96 well ultra-low attachment (ULA) U-bottom plate (Corning® 4515 or Thermo Scientific™ 174925). The plate was centrifuged at 300×g for 5 minutes at 4° C. ECM solution was prepared on ice and all tips and reservoirs were precooled and kept cold during the whole procedure. 50 μl of ECM solution was added to the cell suspension at a final concentration of 2.5% v/v Geltrex™, for a total volume of 100 μl. The plate was again centrifuged at 300×g for 5 minutes at 4° C. The culture was monitored using an IncuCyte® live cell imaging and analysis platform for a maximum of 7 days. 80% of the culture medium was changed with fresh medium every other day. Seeding cells in 50 μl, followed by centrifugation, followed by addition of 50 μl ECM solution may be referred to as an example of an “Protocol B”.

Spheroid Formation—human renal epithelial cells (HREpC)—Protocol B

Protocol B: PromoCell® Human Renal Epithelial Cells (HREpC) were cultured according to the manufacture's protocol “Epithelial Cells—Instruction Manual” (PromoCell GmbH). For spheroid generation, cells were trypsinized (PromoCell® DetachKit C-41200), counted and the cell suspension was seeded at 3,000 or 6,000 cells in 50 μl per well in a 96 well ultra-low attachment (ULA) U-bottom plate (Corning® 4515 or Thermo Scientific™ 174925). The plate was centrifuged at 300×g for 5 minutes at 4° C. ECM solution was prepared on ice and all tips and reservoirs were precooled and kept cold during the whole procedure. 50 μl of ECM solution was added to the cell suspension at a final concentration of 2.5% v/v Geltrex™, for a total volume of 100 μl. The plate was again centrifuged at 300×g for 5 minutes at 4° C. The culture was monitored using an IncuCyte® live cell imaging and analysis platform for a maximum of 7 days. 80% of the culture medium was changed with fresh medium every other day. Seeding cells in 50 μl, followed by centrifugation, followed by addition of 50 μl ECM solution may be referred to as an example of an “Protocol B”.

Spheroid Formation—human coronary artery endothelial cells (HCAEC)—Protocol B

Protocol B: PromoCell® Human Coronary Artery Endothelial Cells (HCAEC) were cultured according to the manufacture's protocol “Endothelial Cells—Instruction Manual” (PromoCell GmbH). For spheroid generation, cells were trypsinized (PromoCell® DetachKit C-41200), counted and the cell suspension was seeded at 3,000 or 6,000 cells in 50 μl per well in a 96 well ultra-low attachment (ULA) U-bottom plate (Corning® 4515 or Thermo Scientific™ 174925). The plate was centrifuged at 300×g for 5 minutes at 4° C. ECM solution was prepared on ice and all tips and reservoirs were precooled and kept cold during the whole procedure. 50 μl of ECM solution was added to the cell suspension at a final concentration of 2.5% v/v Geltrex™, for a total volume of 100 μl. The plate was again centrifuged at 300×g for 5 minutes at 4° C. The culture was monitored using an IncuCyte® live cell imaging and analysis platform for a maximum of 7 days. 80% of the culture medium was changed with fresh medium every other day. Seeding cells in 50 μl, followed by centrifugation, followed by addition of 50 μl ECM solution may be referred to as an example of an “Protocol B”.

Cell Staining—iHH and iHCM

TABLE 1
Harvesting Hepatocytes

| Step | Description |
|---|---|
| 1 | Before use, equilibrate an aliquot of Spheroid Maintenance |
| | Medium and D-PBS to room temperature. |
| 2 | Remove the spent medium from the cell culture plate containing |
| | the hepatocytes. |
| 3 | Wash the hepatocytes once with a volume of D-PBS (Table 2). |
| 4 | Add a volume of Accutase to the cells (Table 2). |
| | Note: Do not use trypsin to dissociate the hepatocytes. |
| 5 | Monitor dissociation with a microscope and incubate the cell |
| | culture plate at room temperature until the cells just begin |
| | to detach from the plate (~2-4 minutes). |
| 6 | Add a rinse volume of Spheroid Maintenance Medium to dilute |
| | the Accutase (Table 2). |
| 7 | Gently pipette up and down several times around the well to |
| | ensure the cells are detached and to generate a cell suspension. |
| | Note: The cell suspension contains mostly small clusters of |
| | cells. Do not attempt to singularize the cells. Over-digestion |
| | of the hepatocytes impairs their functionality in the 3D |
| | spheroid culture. |
| 8 | Transfer the dissociated cells to a 15 ml centrifuge tube. |
| 9 | Add a rinse volume of Spheroid Maintenance Medium (Table 2). |
| | Transfer the medium rinse to the 15 ml centrifuge tube |
| | containing the cells. |
| | Note: Dissociated cells from multiple wells can be pooled |
| | in the same centrifuge tube. |
| 10 | Centrifuge the cell suspension at room temperature at 200 × |
| | g (or ~1,000 rpm) for 3 minutes. |
| 11 | Carefully aspirate the supernatant, taking care not to disturb |
| | the cell pellet. |
| 12 | Gently resuspend the cell pellet in the desired volume of |
| | Spheroid Maintenance Medium depending on the 2D culture |
| | format that was used and the desired target spheroid size (Table 3). |
| | Note: Because the cell suspension contains mostly small |
| | clusters of cells, it is not amenable to counting. The cell |
| | number values listed in Table 3 are estimates based on the |
| | expected recovery numbers provided in Table 2. |

TABLE 2
Summary of Volumes and Measures for Dissociation
(All volumes and measures are per well)

| | Cell Number | | | Volume of |
|---|---|---|---|---|
| Cell Culture | Expected | Volume of | Volume of | Spheroid Maint. |
| Vessel | to be | D-PBS Wash | Accutase | Medium Rinse |
| (2D format) | Recovered | (ml) | (ml) | (ml) |
| 6-well Cell | ~1,000,000 | 2 | 1 | 2 |
| Culture Plate | | | | |
| 12-well Cell | ~400,000 | 1 | 0.5 | 1 |
| Culture Plate | | | | |
| 24-well Cell | ~200,000 | 0.6 | 0.3 | 0.6 |
| Culture Plate | | | | |
| 48-well Cell | ~100,000 | 0.3 | 0.15 | 0.3 |
| Culture Plate | | | | |
| 96-well Cell | ~30,000 | 0.1 | 0.05 | 0.1 |
| Culture Plate | | | | |

TABLE 3
Resuspension Volumes for Cells Harvested from 2D Culture (All volumes are per harvested well)

| | Volume of Spheroid Maintenance Medium (ml) | | | | | |
|---|---|---|---|---|---|---|
| | 100-200 μm | 150-250 μm | 200-300 μm | 250-350 μm | 350-450 μm | 450-550 μm |
| Cell Culture Vessel | Diameter/~500 | Diameter/~1,000 | Diameter/~2,000 | Diameter/~3,000 | Diameter/~5,000 | Diameter/~10,000 |
| 2D Format | Cells | Cells | Cells | Cells | Cells | Cells |
| 6-well Cell Culture Plate | 70 | 35 | 17.5 | 11.67 | 7 | 3.5 |
| 12-well Cell Culture Plate | 28 | 14 | 7 | 4.67 | 2.8 | 1.4 |
| 24-well Cell Culture Plate | 14 | 7 | 3.5 | 2.33 | 1.4 | 0.7 |
| 48-well Cell Culture Plate | 7 | 3.5 | 1.75 | 1.17 | 0.7 | 0.35 |
| 96-well Cell Culture Plate | 2.1 | 1.05 | 0.525 | 0.35 | 0.21 | 0.105 |

iCell Hepatocytes 2.0 (iHH) were cultured according to the manufacture's application protocol “Modeling 3D Liver Tissue: 3D Hepatocyte Spheroids in Low Attachment Plates” (FUJIFILM Cellular Dynamics, Inc). iCell Hepatocytes 2.0 can be harvested from a 2D cell culture plate between days 5 and 9 post-plating for preparing 3D spheroid cultures. Before continuing with “Harvesting Hepatocytes”, iHH were stained with 2.5 μM of CellTracker™ Red CMTPX Dye in serum free Gibco™ DMEM/F-12, no phenol red (Thermo Fisher Scientific) for 20 min at 37° C. and 5% CO₂. Appropriately harvesting hepatocytes from the cell culture plate(s) as described in Table 1 is a critical step to achieve robust functionality upon transfer to a 96 well ultra-low attachment (ULA) U-bottom plate (Corning® 4515 or Thermo Scientific™ 174925) 96-well for 3D spheroid formation. Spheroids were generated as described herein using Table 2 and 3, e.g., Spheroid Formation—iHH—Adjusted Protocol. The culture was monitored using an IncuCyte® live cell imaging and analysis platform for at least 72 h.

iCell® Cardiomyocytes² (iHCM) were thawed according to the manufacture's protocol “User's Guide” (FUJIFILM Cellular Dynamics, Inc) and spheroids were generated as described herein, e.g., Spheroid Formation—iHCM—Adjusted Protocol. Cells were cultured for 4 days for spheroid formation and resulting spheres were subsequently stained with 2.5 μM of CellTracker™ Red CMTPX Dye in iCell® Cardiomyocytes Maintenance Medium for for 20 min at 37° C. and 5% CO₂. The plate was centrifuged at 300×g for 5 minutes, staining solution was removed and medium was changed back to iCell® Cardiomyocytes Maintenance Medium following centrifugation at 300×g for 5 minutes. The culture was monitored using an IncuCyte® live cell imaging and analysis platform for at least 72 h.

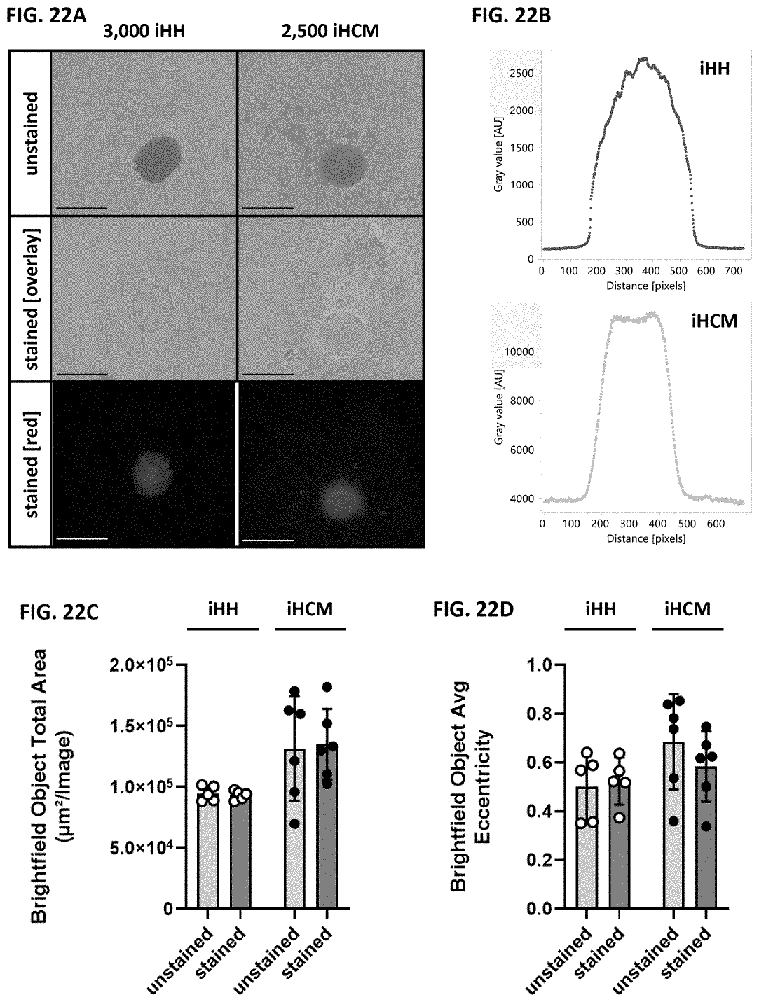

FIGS. 22A-22D show the staining of spheroids generated from iCell® Hepatocytes 2.0 (iHH) and iCell® Cardiomyocytes² (iHCM) using CellTracker™ Red CMTPX Dye. FIG. 22A shows representative images of unstained and stained spheroids after 72 h of staining as brightfield, overlay and red channel exported from the IncuCyte® software. The scale bar indicates 400 μm. FIG. 22B shows the fluorescence signal above background from stained iHH and iHCM cell spheroids using CellTracker™ Red CMTPX Dye calculated as Gray value [AU] using ImageJ (1.53c). FIG. 22C shows the size of unstained and stained iHH and iHCM cell spheroids [Brightfield Object Total Area (μm²/Image)]. FIG. 22D shows the eccentricity of unstained and stained iHH and iHCM cell spheroids 72 h after staining with CellTracker™ Red CMTPX Dye calculated using IncuCyte® software. These results indicate that stained spheroids are comparable in size and eccentricity to unstained spheroids and thus suitable for quantifying biological effects on spheroids using imaging devices, e.g., IncuCyte®.

### Example 19

### Spheroid Formation and Comparison of Ultra-Low Attachment Plates—NCI-111703-RFP, A375-RFP, Hs695T-RFP

Comparison of Spheroid Formation in Four Different Ultra-Low Attachment (ULA) Plates.

NCI-H1703, A375 and Hs695T cells stably expressing red fluorescent protein (RFP) were generated by transducing NCI-H1703, A375 and Hs695T cells with a nucleic acid encoding RFP (NCI-H1703-RFP, A375-RFP, Hs695T-RFP). Cells were seeded according to the common protocol described in Example 1. A final concentration of 2.5% v/v of the extracellular matrix (ECM) reagent Matrigel® (Corning® Matrigel® Basement Membrane Matrix Growth Factor Reduced, Catalog No. 354230) was used. The ECM-cell suspension mixture was seeded in 200 μl per well in four different 96-well ULA U-bottom plates at a final cell concentration of 1e3 (1,000) cells per well for NCI-H1703-RFP, 2e3 (2,000) cells per well for A375-RFP and 1e4 (10,000) cells per well for Hs695T-RFP (plate 1, Corning, 96-well Black/Clear Round Bottom ULA Spheroid Microplate; plate 2, Corning, 96-well Clear Round Bottom ULA Microplate; plate 3, faCellitate, BIOFLOAT™ 96-well plate; plate 4, Thermo Fisher Scientific, Nunclon Sphera 3D culture system). The spheroid cultures were monitored using an IncuCyte® live cell imaging and analysis platform.

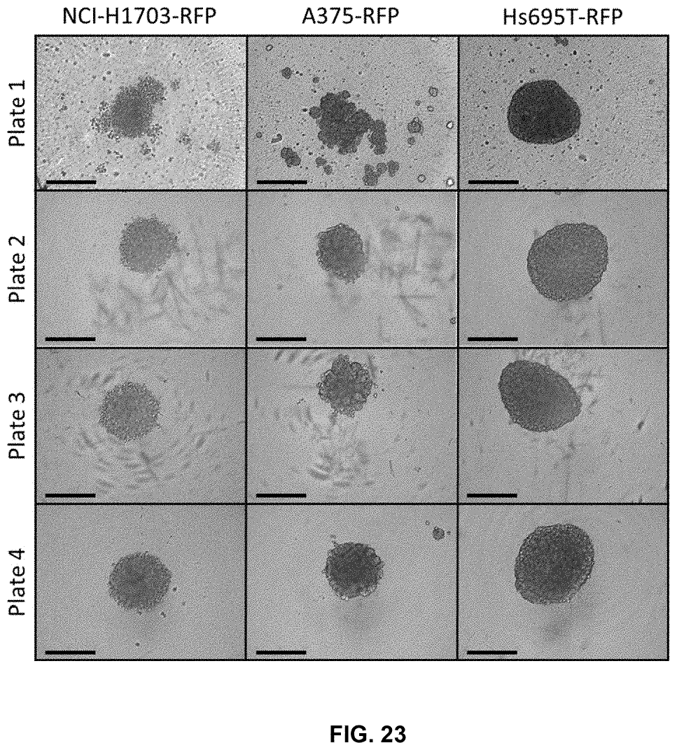

FIG. 23 shows NCI-H1703-RFP, A375-RFP and Hs695T-RFP cell spheroids 72 hours after cell seeding generated using Matrigel®. Cells were seeded in four different types of 96-well ultra-low attachment (ULA) plates. Plate 1, Corning, 96-well Black/Clear Round Bottom ULA Spheroid Microplate; plate 2, Corning, 96-well Clear Round Bottom ULA Microplate; plate 3, faCellitate, BIOFLOAT′ 96-well plate; and plate 4, Thermo Fisher Scientific, Nunclon Sphera 3D culture system. Scale bar 400 μm. Spheroids cultured in plate 1 exhibit irregular shape with satellite spheroids in the surrounding area of the main spheroid. Radial thin scratches/lines are observed extending from the center of the wells to the outside walls of the wells. In plate 2, spheroids grew with a rounder shape compared to that cultured in plate 1 and with fewer satellite spheroids in their periphery. Thick scratches are visible at the bottom of the wells. Spheroids cultured in plate 3 exhibit a round surface for NCI-H1703-RFP and spheroids with an almost round, but slightly fringed surface for A375-RFP. Hs695T-RFP spheroids appear in a rather oval shape. Circular thick scratches are visible at the bottom of the well surrounding the spheroids. In plate 4, spheroids show a compact and round shape for NCI-H1703-RFP and A375-RFP or an almost round shape for Hs695T-RFP with a small number of loose cells or satellite spheroids in the surrounding area of the main spheroid. The background appears clear without any scratches. These results show that culturing tumor cell lines, e.g., NCI-H1703-RFP, A375-RFP and Hs695T-RFP, in plate 4 (Thermo Fisher Scientific, Nunclon Sphera 3D culture system) can yield spheroids that exhibit better round shape, fewer satellite spheroids, and fewer scatches/lines as compared with that culturing in plates 1-3.

### Example 20

### Spheroid Formation and Comparison of Protocols

Comparison of Spheroid Formation Generated by the Common Protocol and the Protocol B for NCI-111703-RFP, Hs695T-RFP, SNU475-RFP, NCI-111792

NCI-H1703, Hs695T and SNU475 cells stably expressing red fluorescent protein (RFP) were generated by transducing NCI-H1703, Hs695T and SNU475 cells with a nucleic acid encoding RFP (NCI-H1703-RFP, Hs695T-RFP and SNU475-RFP). The cell line NCI-H1792 was not transduced with RFP.

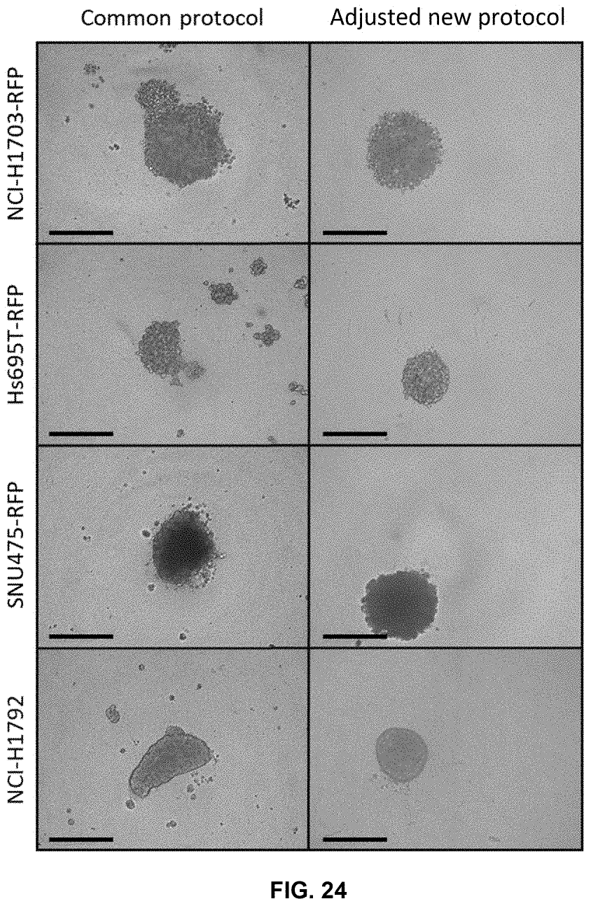

FIG. 24 shows NCI-H1703-RFP, Hs695T-RFP, SNU475-RFP and NCI-H1792 cell spheroids 72 hours after cell seeding generated using Matrigel® with either the common protocol or the Protocol B described herein. Scale bar 400 μm. The left column shows spheroids seeded with the common protocol and the right column shows spheroids seeded with the Protocol B described in Example 1. A final concentration of 2.5% v/v of the extracellular matrix (ECM) reagent Matrigel® (Corning® Matrigel® Basement Membrane Matrix Growth Factor Reduced, Catalog No. 354230) was used.

For the common protocol, the ECM-cell suspension mixture was seeded in 100 μl to 200 μl per well in a 96-well ultra-low attachment (ULA) U-bottom plate (Thermo Fisher Scientific, Nunclon Sphera 3D culture system) at a final cell concentration of 1e3 (1,000) cells per well for NCI-H1703-RFP and NCI-H1792, 2e3 (2,000) cells per well for Hs695T-RFP or 5e3 (5,000) cells per well for SNU475-RFP. The plate was centrifuged at 100×g for 10 minutes at 4° C. The culture monitored using an IncuCyte® live cell imaging and analysis platform.

In Protocol B, the cell suspension (without ECM) was seeded in 50 μl per well in a 96-well ULA U-bottom plate (Thermo Fisher Scientific, Nunclon Sphera 3D culture system) at a final cell concentration of 1e3 (1,000) cells per well for NCI-H1703-RFP, 2e3 (2,000) cells per well for Hs695T-RFP, or 5e3 (5,000) cells per well for SNU475-RFP and NCI-H1792. The plate was centrifuged at 100×g for 10 minutes at 4° C. 50 μl ECM reagent Matrigel® (Corning® Matrigel® Basement Membrane Matrix Growth Factor Reduced, Catalog No. 354230) was added to the cell suspension at a final concentration of 2.5% v/v. The plate was again centrifuged at 100×g for 10 minutes at 4° C. The culture was monitored using an IncuCyte® live cell imaging and analysis platform. FIG. 24 shows that spheroids produced by Protocol B exhibit rounder shape with a smoother surface area and with less to no satellite spheroids as compared to that produced by the common protocol. In contrast, spheroids produced by the common protocol exhibit irregular shape with a fringed surface area and several satellite spheroids surrounding the main spheroid.

TCR-Electroporated T Cell-Mediated Killing of Spheroids Generated Using Protocol B—NCI-1703-RFP

Tumor spheroids were generated using the NCI-H1703 tumor cell line expressing RFP. Spheroid formation was induced using Protocol B (as described in Example 1 modified as in Protocol B set forth in Example 5. Matrigel® 2.5% v/v was used. CD8 T cells were prepared using peripheral blood mononuclear cells (PBMC's) from one healthy donor. T cells were pre-activated using OKT3 and CD28 stimulation. After three days, cells were electroporated with TCR mRNA of interest resulting in transient expression of the TCR. The co-culture was initiated after 5 days of spheroid growth. Electroporated T cells were added at a ratio of, e.g., 10:1, to the target NCI-H1703 cells. Tumor cell proliferation was measured using the IncuCyte by analyzing the red fluorescence area over time.

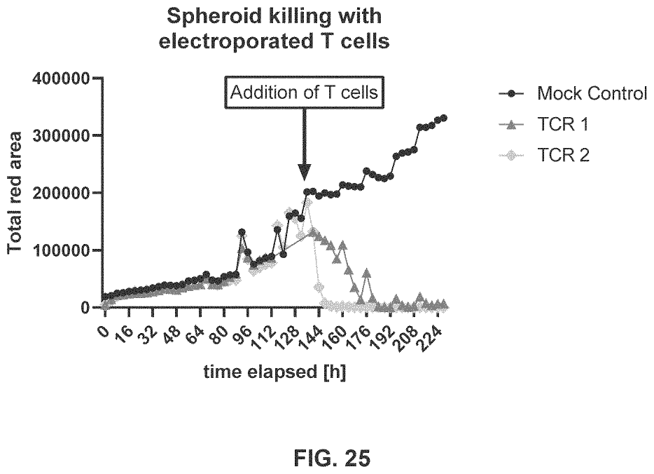

For example, the RFP-transduced NCI-H1703 cells were grown in spheroid formation using Protocol B. Primary CD8 T cells were either electroporated with mock RNA or with TCR mRNA (TCR 1 and 2) encoding a specific TCR recognizing a peptide antigen on the NCI-H1703. Total red area against time is plotted. FIG. 25 shows NCI-H1703 cell spheroid killing by electroporated T cells. There is no reduction of total red area of NCI-H1703 spheroid by mock control T cells. The reduction of total red area of NCI-H1703 spheroid by T cells expressing TCR1 or TCR2 indicates target-specific tumor cell killing. These results suggests that spheroids produced by Protocol B can be readily used to measure cytotoxic activity of T cell products for adoptive cellular therapy.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific embodiments of this disclosure set forth in the appended claims. The foregoing embodiments are presented by way of example only; the scope of the present disclosure is to be limited only by the following claims.

Unless otherwise specified herein, ranges of values set forth herein are intended to operate as a scheme for referring to each separate value falling within the range individually, including but not limited to the endpoints of the ranges, and each separate value of each range set forth herein is hereby incorporated into the specification as if it were individually recited.

This specification may include references to “one embodiment”, “an embodiment”, “embodiments”, “one aspect”, “an aspect”, or “aspects”. Each of these words and phrases is not intended to convey a different meaning from the other words and phrases. These words and phrases may refer to the same embodiment or aspect, may refer to different embodiments or aspects, and may refer to more than one embodiment or aspect. Various embodiments and aspects may be combined in any manner consistent with this disclosure.

1. A method for generating cellular spheroids, comprising:
(a) providing cells capable of spheroid formation;
(b) centrifuging the cells;
(c) adding extracellular matrix to the cells of (b) to produce a cell suspension comprising a desired concentration of extracellular matrix; and
(d) allowing at least one spheroid to form;
wherein (b) is performed before (c).

2. The method of claim 1, further comprising centrifuging the cell suspension of (c) before performing (d).

3. The method of claim 1, wherein the cells comprise primary cells, tumor cells, immortalized cells, or combinations thereof.

4. The method of claim 1, further comprising seeding the cells in a cell culture apparatus prior to performing (b).

5. (canceled)

6. The method of claim 4, wherein the cell culture apparatus comprises at least one partition, wherein the at least one spheroid comprises only one spheroid in the at least one partition.

7. The method of claim 1, wherein the extracellular matrix comprises laminin, collagen IV, heparin sulfate proteoglycans, nidogen, or combinations thereof.

8. The method of claim 1, wherein at the at least one spheroid comprises a more uniform morphology, a more compact structure, a lower level of shape eccentricity, more uniformity of compactness, more uniformity of size, or a combination thereof, as compared to a spheroid generated without performing (b) before (c).

9. The method of claim 1, wherein the at least one spheroid comprises a lower amount of cellular debris, a lower number of non-spheroid cellular aggregates, or a combination thereof, as compared to a spheroid generated without performing (b) before (c).

10.-11. (canceled)

12. The method of claim 1, wherein the at least one spheroid has a circularity of approximately 0.1 to approximately 1.0, a circularity of approximately 0.1 to approximately 1.0, a circularity of approximately 0.2 to approximately 1.0, a circularity of approximately 0.4 to approximately 1.0, a circularity of approximately 0.5 to approximately 1.0, a circularity of approximately 0.6 to approximately 1.0, a circularity of approximately 0.7 to approximately 0.95, a circularity of approximately 0.7 to approximately 0.9, a circularity of approximately 0.8 to approximately 0.85, a circularity of at least approximately 0.3, a circularity of at least approximately 0.4, a circularity of at least approximately 0.45, a circularity of at least approximately 0.5, a circularity of at least approximately 0.55, a circularity of at least approximately 0.6, a circularity of at least approximately 0.65, a circularity of at least approximately 0.7, a circularity of at least approximately 0.75, a circularity of at least approximately 0.8, a circularity of at least approximately 0.85, a circularity of at least approximately 0.90, a circularity of at least approximately 0.95, a circularity of at least approximately 0.97, a circularity of at least approximately 0.98, a circularity of at least approximately 0.99, or a circularity of approximately 1.

13. The method of claim 1, wherein the at least one spheroid has an eccentricity approximately 0 to approximately 0.7, an eccentricity of approximately 0.6 to approximately 0.75, an eccentricity of approximately 0.5 to approximately 0.95, an eccentricity of approximately 0.5 to approximately 0.9, an eccentricity of approximately 0.4 to approximately 0.85, an eccentricity of approximately 0.3 to approximately 0.7, an eccentricity of approximately 0.2 to approximately 0.65, an eccentricity of approximately 0.1 to approximately 0.5, an eccentricity of approximately 0.1 to approximately 0.4, an eccentricity of approximately 0.1 to approximately 0.3, an eccentricity of approximately 0.1 to approximately 0.2, an eccentricity of approximately 0.1, an eccentricity of approximately 0.05, an eccentricity of approximately 0.02, an eccentricity of approximately 0.01, an eccentricity of approximately 0, an eccentricity of approximately 0.7 or less, an eccentricity of approximately 0.65 or less, an eccentricity of approximately 0.6 or less, an eccentricity of approximately 0.55 or less, an eccentricity of approximately 0.5 or less, an eccentricity of approximately 0.45 or less, an eccentricity of approximately 0.4 or less, an eccentricity of approximately 0.35 or less, an eccentricity of approximately 0.3 or less, an eccentricity of approximately 0.25 or less, an eccentricity of approximately 0.2 or less, an eccentricity of approximately 0.15 or less, an eccentricity of approximately 0.1 or less, an eccentricity of approximately 0.05 or less, an eccentricity of approximately 0.02 or less, or an eccentricity of approximately 0.01 or less.

14.-30. (canceled)

31. A method for generating cellular spheroids, comprising:
(a) providing cells capable of spheroid formation in a first volume of media that is a first fraction of a testing volume desired for characterizing, analyzing, challenging, otherwise testing, of at least one spheroid, or a combination thereof;
(b) centrifuging the cells;
(c) adding to the cells of (b) extracellular matrix in a second volume that is a second fraction of the testing volume to produce a cell suspension comprising a desired concentration of extracellular matrix; and
(d) allowing at least one spheroid to form;
wherein (b) is performed before (c).

32. The method of claim 31, further comprising centrifuging the cell suspension of (c) before performing (d).

33. The method of claim 31, wherein the cells comprise tumor cells, immortalized cells, primary cells, or combinations thereof.

34. The method of claim 31, further comprising seeding the cells in a cell culture apparatus prior to performing (b).

35. The method of claim 31, further comprising adding to the at least one spheroid of (d) media and/or a control and/or a test composition in a third volume sufficient to produce the testing volume.

36. (canceled)

37. The method of claim 34, wherein the cell culture apparatus comprises at least one partition, wherein the at least one spheroid comprises only one spheroid in the at least one partition.

38. The method of claim 31, wherein the extracellular matrix comprises laminin, collagen IV, heparin sulfate proteoglycans, nidogen, or combinations thereof.

39. The method of claim 31, wherein at the at least one spheroid comprises a more uniform morphology, a more compact structure, a lower level of shape eccentricity, more uniformity of compactness, more uniformity of size, or a combination thereof, as compared to a spheroid generated without performing (b) before (c).

40.-42. (canceled)

43. The method of claim 31, wherein the at least one spheroid has a circularity of approximately 0.1 to approximately 1.0, a circularity of approximately 0.1 to approximately 1.0, a circularity of approximately 0.2 to approximately 1.0, a circularity of approximately 0.4 to approximately 1.0, a circularity of approximately 0.5 to approximately 1.0, a circularity of approximately 0.6 to approximately 1.0, a circularity of approximately 0.7 to approximately 0.95, a circularity of approximately 0.7 to approximately 0.9, a circularity of approximately 0.8 to approximately 0.85, a circularity of at least approximately 0.3, a circularity of at least approximately 0.4, a circularity of at least approximately 0.45, a circularity of at least approximately 0.5, a circularity of at least approximately 0.55, a circularity of at least approximately 0.6, a circularity of at least approximately 0.65, a circularity of at least approximately 0.7, a circularity of at least approximately 0.75, a circularity of at least approximately 0.8, a circularity of at least approximately 0.85, a circularity of at least approximately 0.90, a circularity of at least approximately 0.95, a circularity of at least approximately 0.97, a circularity of at least approximately 0.98, a circularity of at least approximately 0.99, or a circularity of approximately 1.

44. The method of claim 31, wherein the at least one spheroid has an eccentricity approximately 0 to approximately 0.7, an eccentricity of approximately 0.6 to approximately 0.75, an eccentricity of approximately 0.5 to approximately 0.95, an eccentricity of approximately 0.5 to approximately 0.9, an eccentricity of approximately 0.4 to approximately 0.85, an eccentricity of approximately 0.3 to approximately 0.7, an eccentricity of approximately 0.2 to approximately 0.65, an eccentricity of approximately 0.1 to approximately 0.5, an eccentricity of approximately 0.1 to approximately 0.4, an eccentricity of approximately 0.1 to approximately 0.3, an eccentricity of approximately 0.1 to approximately 0.2, an eccentricity of approximately 0.1, an eccentricity of approximately 0.05, an eccentricity of approximately 0.02, an eccentricity of approximately 0.01, an eccentricity of approximately 0, an eccentricity of approximately 0.7 or less, an eccentricity of approximately 0.65 or less, an eccentricity of approximately 0.6 or less, an eccentricity of approximately 0.55 or less, an eccentricity of approximately 0.5 or less, an eccentricity of approximately 0.45 or less, an eccentricity of approximately 0.4 or less, an eccentricity of approximately 0.35 or less, an eccentricity of approximately 0.3 or less, an eccentricity of approximately 0.25 or less, an eccentricity of approximately 0.2 or less, an eccentricity of approximately 0.15 or less, an eccentricity of approximately 0.1 or less, an eccentricity of approximately 0.05 or less, an eccentricity of approximately 0.02 or less, or an eccentricity of approximately 0.01 or less.

45.-61. (canceled)
